(19) United States
(12) Patent Application Publication
(10) Pub. No.: US 2008/0281094 A1
(43) Pub. Date: Nov. 13, 2008

(54) Regioselective Functionalisation and Protection of Spirolactams

(75) Inventors: Pedro Noheda Marin, Madrid (ES); Manuel Bernabe Pajares, Madrid (ES); Sergio Maroto Quintana, Mardrid (ES); Nuria Tabares Cantero, Madrid (ES); Raul Benito Arenas, Burgos (ES)

Correspondence Address: MORGAN & FINNEGAN, L.L.P., 3 WORLD FINANCIAL CENTER, NEW YORK, NY, 10281-2101, US

(73) Assignee: LABORATORIOS DEL DR. ESTEVE, S.A., Barcelona (ES)

(21) Appl. No.: 11/568,902

(22) PCT Filed: May 10, 2005
(86) PCT No.: PCT/EP2005/005149

§ 371 Date: Oct. 9, 2007

(30) Foreign Application Priority Data
May 10, 2004 (EP) 04380104.2
May 10, 2004 (ES) P200401123
May 14, 2004 (US) 10/846466
May 20, 2004 (EP) 04076477.1
May 25, 2004 (US) 10/853639
May 27, 2004 (ES) P200401285
Dec. 30, 2004 (EP) 04380295.8
Feb. 1, 2005 (US) 11/047860

(51) Int. Cl. C07D 205/04

(52) U.S. Cl. 540/355

(57) ABSTRACT

The invention provides highly functionalised spiro-fused lactams of en formula (I) having a cyclohexane moiety with the desired number of protected or un-protected functional groups or carbonated structures, which are introduced with high stereo and regioselectivity, as well as processes for their obtention. These compounds are useful for the synthesis of abroad range of bioactive molecules, such as condoritols and aminoinositols and their analogues.

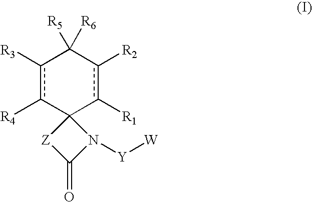

### FIELD OF THE INVENTION

The present invention relates to new regioselectively hydroxylated, protected and functionalized spirolactams and to processes for their synthesis.

### BACKGROUND OF THE INVENTION

Lactams are compounds of high interest due to their biological activities, for example well known β-lactams such as some penicillins, cephalosporins and carbapenems have antibacterial activity.

Spirolactams are one particular class of lactams that have shown interesting biological properties. Some spiro-fused azetidinones have been described as having antibacterial activity, see U.S. Pat. No. 4,680,388, or hypocholesterolemic properties, see for example WO 94 17038. Additionally, if these compounds have the adequate functionality they are valuable intermediates towards different families of compounds. The spirolactam ring is the equivalent of an alpha amino or hydroxy aminoacid and opens many possibilities in diastero and/or enantioselective synthesis.

Miyazawa, E. et al. in Heterocycles, vol 59, 1:149-160 “Synthesis of spiro-fused nitrogen heterocyclic compounds via N-methoxy-N-acylnitrenium ions using phenyliodine (III) bis(trifluoroacetate) in trifluoroethanol” describe a process to obtain functionalised spirolactams including some spirodienones.

Kukugawa, Y. e al. in J. Org. Chem. 2003, vol. 68, 6739-6744 “Intramolecular cyclization with nitrenium ions generated by treatment of N-acylaminophthalimides with hypervalent iodine compounds: formation of lactams and spirofused lactams” describes the formation of functionalised spirolactams having diene and dienone funcionalities.

The conduritols, aminoconduritols, aminoinositols and their derivatives also possess interesting biological properties, some of them have been shown as being antitumoral and antibiotic. Although some synthetic processes exist for these compounds (See Yong-Uk Kwon et al, J. Org. Chem. 2002, v1. 67, 3327-3338 “Facile syntheses of all possible diastereomers of conduritol and various derivatives of inositol stereoisomers in high enantiopurity from inyo-inositol”), there are still difficulties to obtain these compounds or corresponding analogues.

As it is apparent from the above, any efficient process for producing functionalised spirolactam compounds in high yield, with various functionalities, introduced in a controlled and regioselective manner, would be a welcome contribution to the art and will open the door to a variety of biologically active compounds.

### SUMMARY OF THE INVENTION

Starting from the compounds described in our applications EP 04380104.2, EP04076477.1, and PCT/EP2005/ . . . (filed the same day as the present application), we found a basic set of processes that allows the controlled production of very stable, highly functionalised, spiro-fused lactames which are useful as intermediate compounds in the preparation of a variety of chemical structures, including, if necessary, by means of chimio-, loco-, regio-, diastero- and/or enantioselective processes. Additional carbon structures can be incorporated at the desired positions by means of simple reactions, generating new intermediates of interest.

In one aspect the invention provides a compound of formula I:

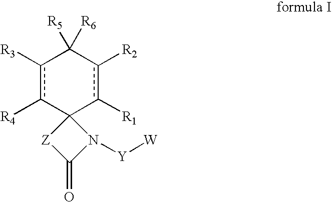

wherein R₁, R₂, R₃ and R₄ are each independently selected from H, OH, halo, OPR, ═O, cyano, substituted or unsubstituted alkyl, substituted or unsubstituted aryl, substituted or unsubstituted heterocyclyl, substituted or unsubstituted alkoxy, substituted or unsubstituted aryloxy, substituted or unsubstituted amino or halogen,

R₅ and R₆ together are ═O or —O—(CH₂)ₘ—, wherein m is selected from 1, 2, 3, 4 or 5; or R₅ is selected from H, OH, OPR and R₆ is selected from hydrogen, cyano, substituted or unsubstituted alkyl, substituted or unsubstituted cycloalkyl, substituted or unsubstituted alkenyl, substituted or unsubstituted alkinyl, substituted or unsubstituted aryl, substituted or unsubstituted heterocyclyl,

with the proviso that at least one of R₁, R₂, R₃, R₄ or R₅ is OH, substituted or unsubstituted alkoxy, substituted or unsubstituted aryloxy or OPR;

PR is an hydroxyl protecting group that can be the same or different on each of R₁, R₂, R₃, R₄ or R₅ and that can simultaneously protect 1, 2 or 3 hydroxy groups;

the dotted line represents a single or double bond, with the proviso that when both R₁ and R₂ or R₃ and R₄ are H then there is a double bond between the two C to which the H are linked;

Z is —(CRaRb)ₙ— or —CH₂—(CRaRb)— or —(CRaRb)—CH₂— or —CH₂—(CRaRb)—CH₂—, or —(CH₂)₂—(CRaRb)— or —(CRaRb)-(CH₂)₂— wherein n is a number selected from 1, 2 or 3 and Ra and Rb are each independently selected from hydrogen, substituted or unsubstituted alkyl, substituted or unsubstituted cycloalkyl, substituted or unsubstituted alkenyl, substituted or unsubstituted aryl, substituted or unsubstituted heterocyclyl, substituted or unsubstituted alkoxy, substituted or unsubstituted aryloxy, substituted or unsubstituted amino or halogen;

Y is selected from —O—, —S—, —NRa— or —C(O)—, wherein Ra is as previously defined;

W is a group comprising at least a group selected from substituted or unsubstituted aryl, substituted or usubstituted heterocyclyl, substituted or unsubstituted alkenyl; or a salt, complex or solvate thereof

In one embodiment the compounds of the invention are as defined with the proviso that when Z is —CH₂CH₂— then Y is selected from —O—, —S—, —NRa— or —C(O)—.

In one embodiment we prefer that n is 1. In this case Z is preferably —CHRa—.

In another embodiment W is selected from substituted or unsubstituted arylalkyl, substituted or unsubstituted heterocyclylalkyl, substituted or unsubstituted alkenyl. More preferably it is arylalkyl, preferably benzyl.

In a further embodiment Y is preferably —O—.

The invention also provides for a process for the preparation of a compound according of formula I, which comprises in any order one or more of a step selected from the group consisting of:

a) hydroxylation, ketohydroxylation or dihydroxylation
b) hydroxyl or carbonyl protection
c) nucleophilic attack at the carbonyl group or double bonds
d) hydroxyl inversion
e) allylic rearrangements
applied to a compound of formula IV:

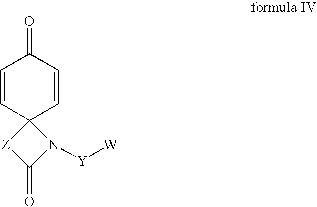

wherein Z, Y and W are as defined above.

### DETAILED DESCRIPTION OF THE INVENTION

In this description the following numbering will be used:

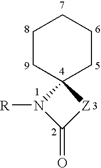

In one aspect the invention provides compounds of formula I as above defined. In the compounds of formula I, the group Z gives rise to a ring of 4, 5 or 6 members.

Although rings of 5 or 6 are also comprised within the scope of the invention, in one embodiment the β-lactam ring (n=1) is preferred because of the further uses that can be given to such compounds.

Substitution on position Z creates a stereogenic center that could induce selective functionalisation on the benzodienone moiety. In a preferred embodiment Z has a chiral center. Especially preferred is —CHRa— with Ra not being H, in these cases the stereogenic center in the β-lactam ring allows for the selectivity or specificity of any further reaction. More preferably Ra is an halo, hydroxy, alkoxy, aryloxy group or an hydroxy protected group.

The group Y in the compounds of formula I plays a role in the stability and conformation. In an embodiment Y is preferably —O—, although other atoms are not excluded as long as the final product is stable.

The W group is important for the stabilization of the compounds of formula I. It comprises unsaturated bonds or aromatic groups to increase the p interaction between W and the double bonds or hydrogens.

Preferably W is selected from substituted or unsubstituted arylalkyl, substituted or unsubstituted heterocyclylalkyl, substituted or unsubstituted alkenyl.

More preferably it is arylalkyl, preferably benzyl.

In a particular embodiment, W is —CRaRb-Q or —SiRaRb-Q since the stability of the conformation is further improved by the presence of a —CRaRb— or a —SiRaRb— linker between Y and the substituent Q which has p (pi) interactions with the benzodienone moiety. The linker is preferably —CHRa—. This introduces a chiral center if necessary, and will advantageously open the way to diastero- and/or enantioselective synthesis in addition to the selection for one face which is explained below. Depending on the size of Ra it can also modulate the p (pi) interactions.

In one embodiment W is an aralkyl group. Among the aryl groups substituted or unsubstituted phenyl and naphthyl are preferred. Heterocyclylalkyl groups are also envisaged. Benzyl is the simplest W subtituent and gives good results. Particular embodiments of the compounds of the present invention are as follows. A compound as defined above having formula II

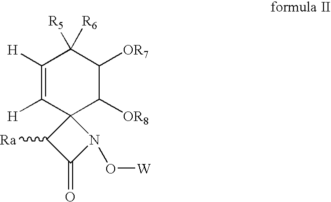

wherein R₇ and R₈ are independently selected from H, substituted or unsubstituted alkyl or PR; W, Ra, R₅ and R₆ are as defined above, or their salts, isomers or solvates.

In another embodiment a compound having formula III is preferred,

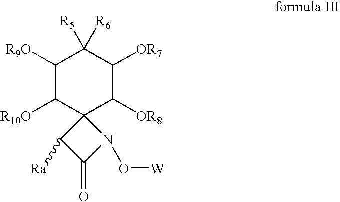

wherein R₇, R₈, R₉ and R₁₀ are each independently selected from H, substituted or unsubstituted alkyl or PR; W, Ra, R₅ and R₆ are as defined above. Particular embodiments of the invention are compounds that correspond to any of the following formulae:

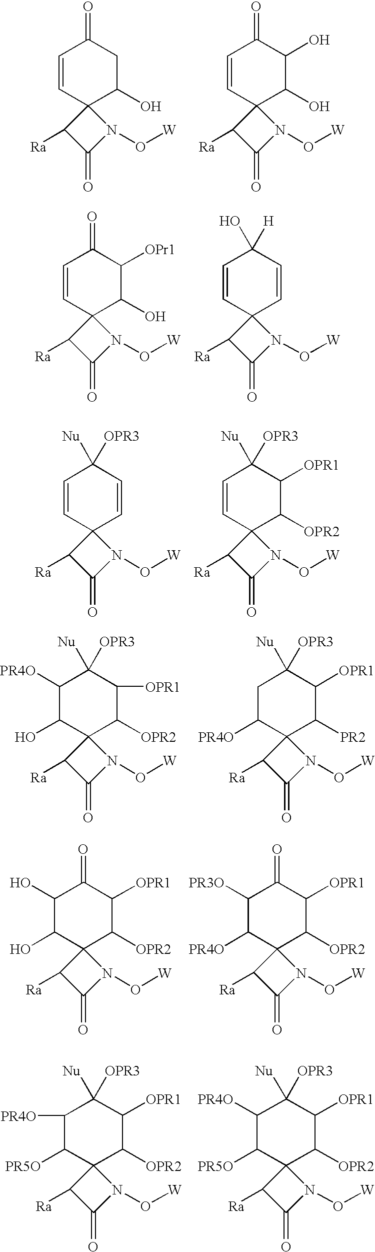

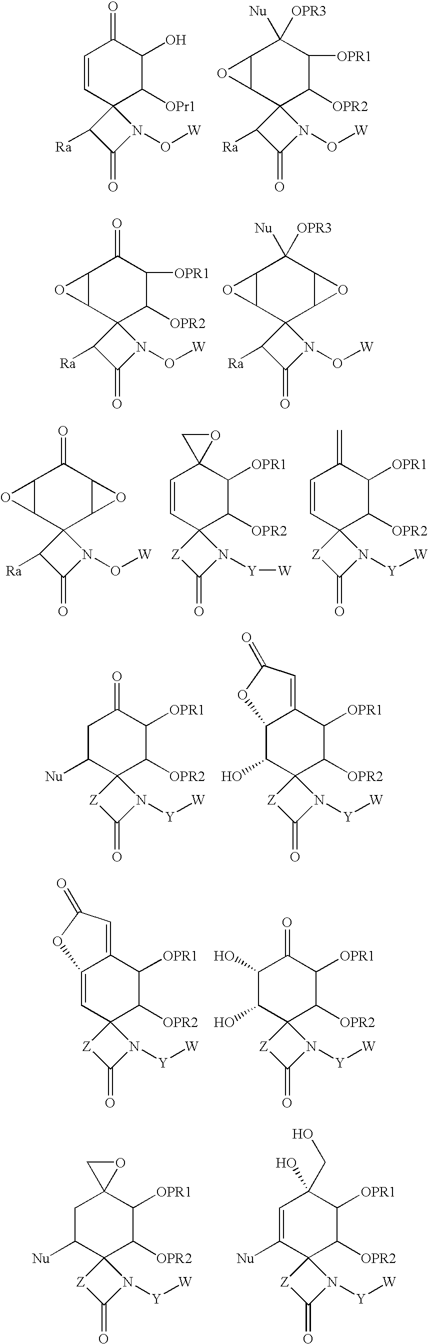

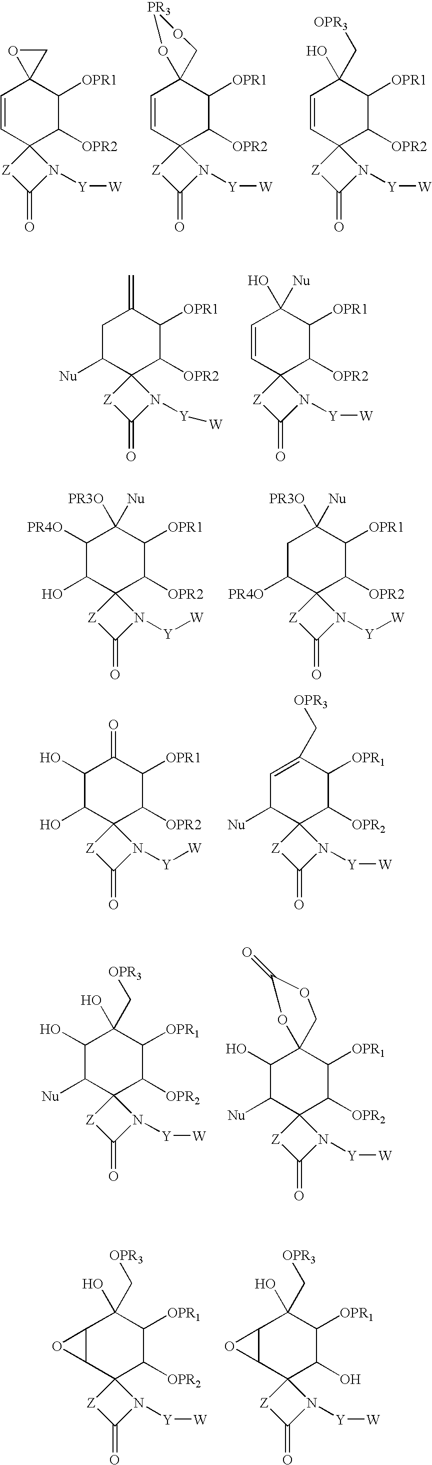

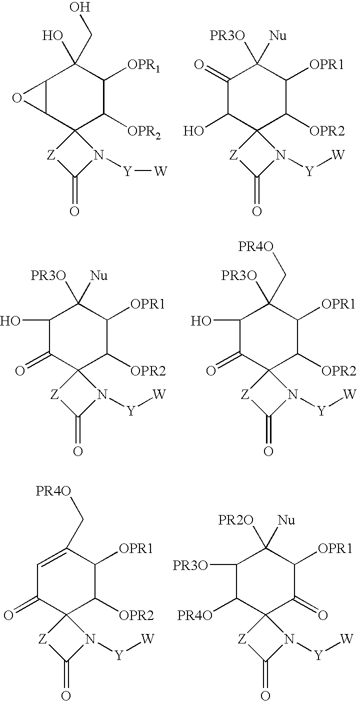

wherein W, PR and Ra are as above defined and wherein the protecting groups PR1-5 can be the same or different and can simultaneously protect 2 or 3 different hydroxy groups, and Nu is a nucleophilic group; their diastereoisomers, enantiomers and mixtures thereof. Representative compounds are those having the following relative configurations:

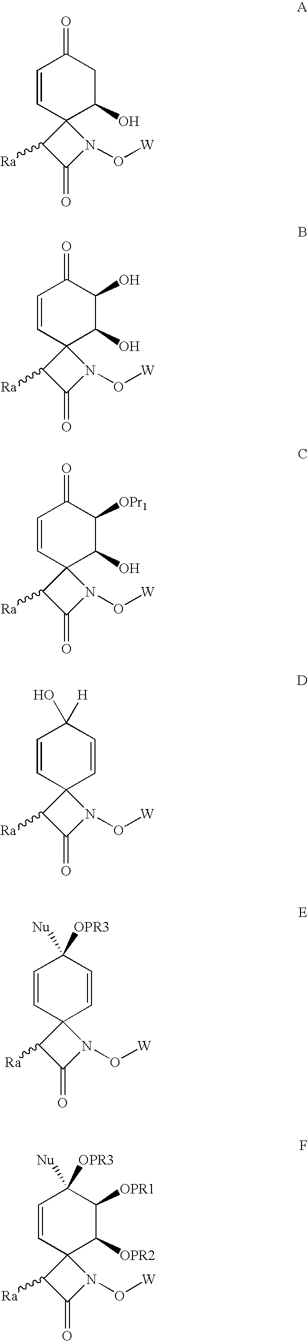

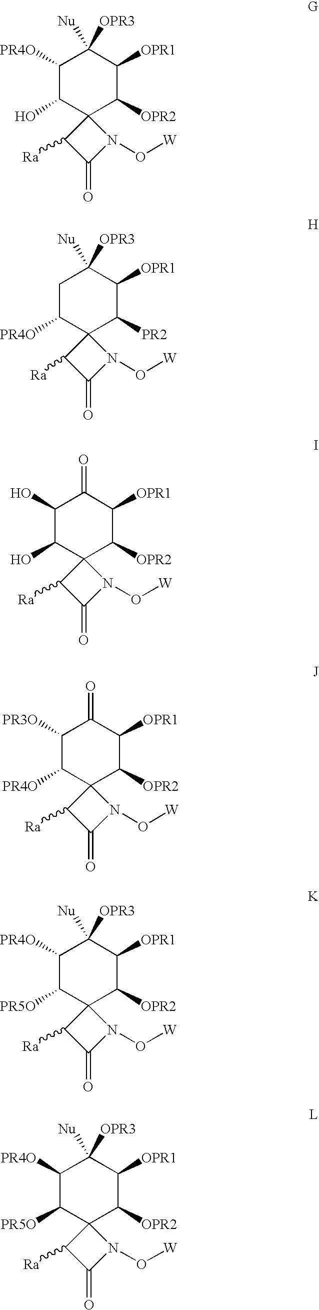

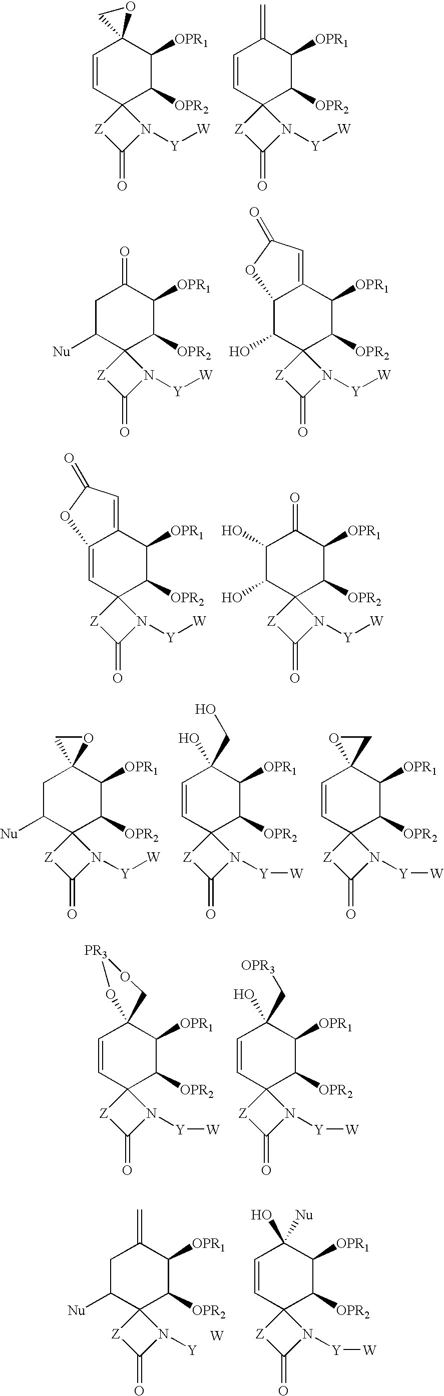

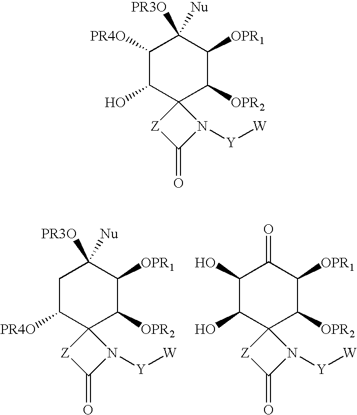

Further embodiments of the compounds of the invention are described below and in the examples. It is evident for the person skilled in the art that many variations and configurations are possible and can be obtained at will depending on the compounds selected as starting materials, the relative configurations of the functional groups present, the presence or not of chiral centers, and on the combination of reactions that are applied. The present invention encompasses all such variations and possibilities.

In the above definition of compounds of formula (I) and in the description the following terms have the meaning indicated:

“Alkyl” refers to a straight or branched hydrocarbon chain radical consisting of carbon and hydrogen atoms, containing no saturation, having 1-12, preferably one to eight carbon atoms, and which is attached to the rest of the molecule by a single bond, e.g., methyl, ethyl, n-propyl, i-propyl, n-butyl, t-butyl, n-pentyl, etc. Alkyl radicals may be optionally substituted by one or more substituents such as halo, hydroxy, alkoxy, carboxy, cyano, carbonyl, acyl, alkoxycarbonyl, amino, nitro, mercapto and alkylthio.

“Alkoxy” refers to a radical of the formula —ORa where Ra is an alkyl radical as defined above, e.g., methoxy, ethoxy, propoxy, etc. “Aryloxy” refers to a radical of formula —ORb wherein Rb is an aryl radical as defined below.

“Amino” refers to a radical of the formula-NH₂, —NHRa, —NRaRb.

“Aryl” refers to an aromatic hydrocarbon radical such as phenyl, naphthyl or anthracyl. The aryl radical may be optionally substituted by one or more substituents such as hydroxy, mercapto, halo, alkyl, phenyl, alkoxy, haloalkyl, nitro, cyano, dialkylamino, aminoalkyl, acyl and alkoxycarbonyl, as defined herein.

“Aralkyl” refers to an aryl group linked to an alkyl group such as benzyl and phenethyl.

“Cycloalkyl” refers to a saturated carbocyclic ring having from 3 to 8 carbon atoms.

“Heterocyclyl” refers to a stable 3- to 15-membered ring which consists of carbon atoms and from one to five heteroatoms selected from the group consisting of nitrogen, oxygen, and sulfur, preferably a 4- to 8-membered ring with one or more heteroatoms, more preferably a 5- or 6-membered ring with one or more heteroatoms. For the purposes of this invention, the heterocycle may be a monocyclic, bicyclic or tricyclic ring system, which may include fused ring systems; and the nitrogen, carbon or sulfur atoms in the heterocyclyl radical may be optionally oxidised; the nitrogen atom may be optionally quaternized; and the heterocyclyl radical may be partially or fully saturated or aromatic. Examples of such heterocycles include, but are not limited to, azepines, benzimidazole, benzothiazole, furan, isothiazole, imidazole, indole, piperidine, piperazine, purine, quinoline, thiadiazole, tetrahydrofuran.

“Hydroxyl protecting group” refers to a group that blocks the OH function for further reactions and can be removed under controlled conditions. The hydroxyl protecting groups are well known in the art, representative protecting groups are silyl ethers such as trimethylsilyl ether, triethylsilyl ether, tert-butyldimethylsilyl ether, tert-butyldiphenylsilyl ether, tri-isopropylsilyl ether, diethylisopropylsilyl ether, thexyldimethylsilyl ether, triphenylsilyl ether, di-tert-butylmethylsilyl ether; alkyl ethers such as methyl ether, tert-butyl ether, benzyl ether, p-methoxybenzyl ether, 3,4-dimethoxybenzyl ether, trityl ether; allyl ether; alkoxymethyl ether such as methoxymethyl ether, 2-methoxyethoxymethyl ether, benzyloxymethyl ether, p-methoxybenzyloxymethyl ether, 2-(trimethylsilyl)ethoxymethyl ether; tetrahydropyranyl and related ethers; methylthiomethyl ether; Esters such as acetate ester, benzoate ester; pivalate ester; methoxyacetate ester; chloroacetate ester; levulinate ester; Carbonates such as benzyl carbonate, p-nitrobenzyl carbonate, tert-butyl carbonate, 2,2,2-trichloroethyl carbonate, 2-(trimethylsilyl)ethyl carbonate, allyl carbonate; and sulphates such as SO₃.py. Additional examples of hydroxyl protecting groups can be found in reference books such as Greene and Wuts' “Protective Groups in Organic Synthesis”, John Wiley & Sons, Inc., New York, 1999. References herein to substituted groups in the compounds of the present invention refer to the specified moiety that may be substituted at one or more available positions by one or more suitable groups, e.g., halogen such as fluoro, chloro, bromo and iodo; cyano; hydroxyl; nitro; azido; alkanoyl such as a C₁₋₆ alkanoyl group such as acyl and the like; carboxamido; alkyl groups including those groups having 1 to about 12 carbon atoms or from 1 to about 6 carbon atoms and more preferably 1-3 carbon atoms; alkenyl and alkynyl groups including groups having one or more unsaturated linkages and from 2 to about 12 carbon or from 2 to about 6 carbon atoms; alkoxy groups having one or more oxygen linkages and from 1 to about 12 carbon atoms or I to about 6 carbon atoms aryloxy such as phenoxy; alkylthio groups including those moieties having one or more thioether linkages and from 1 to about 12 carbon atoms or from 1 to about 6 carbon atoms; alkylsulfinyl groups including those moieties having one or more sulfinyl linkages and from 1 to about 12 carbon atoms or from 1 to about 6 carbon atoms; alkylsulfonyl groups including those moieties having one or more sulfonyl linkages and from 1 to about 12 carbon atoms or from 1 to about 6 carbon atoms; aminoalkyl groups such as groups having one or more N atoms and from 1 to about 12 carbon atoms or from 1 to about 6 carbon atoms; carbocylic aryl having 6 or more carbons, particularly phenyl or naphthyl and aralkyl such as benzyl. Unless otherwise indicated, an optionally substituted group may have a substituent at each substitutable position of the group, and each substitution is independent of the other.

The compounds of the invention can be prepared using a basic set of simple reactions that allow the protection, functionalisation of the different positions in a very stereospecific manner. These procedures will be explained below. In the following note that the configurations given are only relative configurations and not that of the pure enantiomers. To proceed with enantioselectivity, either a chiral center such as in Z or W is needed, or the use of chiral reagents.

In our applications EP04380104.2, and PCT/EP2005/ . . . (filed the same day as the present application), which are incorporated herein by reference in their entirety, we describe new compounds having a formula IV and processes for their obtention:

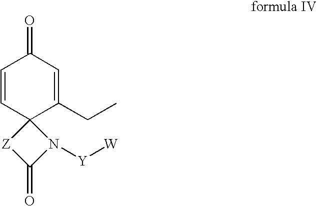

wherein Z, W and Y are as above defined and W is a group with sufficient electronic density to stabilize the compound through p (pi) interactions with the benzodienone moiety, preferably a group having unsaturated bonds or aromatic groups, more preferably it is selected from substituted or unsubstituted arylalkyl, substituted or unsubstituted heterocyclylalkyl, or substituted or unsubstituted alkenyl.

These compounds are remarkably stable due to p interactions between the W group and the benzodienone moiety. Additionally these compounds adopt a preferential conformation in which the W group blocks one of the faces of the benzodienone (hereinafter the β face) and is “fixed” there by the p interactions, directing further reactions to the free face of the benzodienone moeity (hereinafter the a face).

Taking advantage of this, we have found that starting from these compounds it is possible to regioselectively hydroxylate, functionalise and protect the different positions of the benzodienone group, to give a broad range of stable compounds having the desired functionality and protection at each of the 5 available positions. of the cyclohexadienone moiety. Further, carbonated substituents can be introduced on the obtained compounds, to generate a new carbon skeleton at wish. All these highly functionalised compounds are useful as building blocks for a wide variety of bioactive compounds.

If only one hydroxy group is desired, it can be introduced regioselectively for example via formation of the cyanhydrine on the carbonyl group, the hydroboration or hydrosililation of one of the double bonds (via the a face), then oxidation and final treatment with acid or AgF:

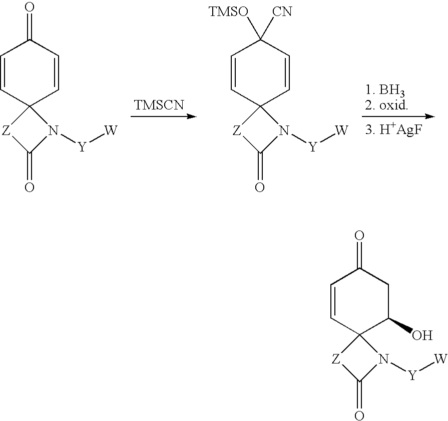

Other alternatives procedures are possible. Significantly, the hydroxylation takes place via one face of the dienone only. The hydroxyl group can then be protected with any desired hydroxyl protecting group such as those listed above.

In another embodiment of the invention we provide for the diastereoselective dihydroxylation of one of the double bonds of the benzodienone moiety. This is a surprising result, in view of the expected poor reactivity of the structure of formula IV, due to the highly deactivated double bonds. Additionally, contrary to what is expected, the dihydroxylation takes place regioselectively, only via the a face:

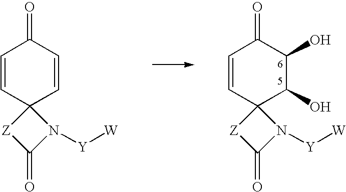

This oxidation occurs readily under mild conditions, such as using OsO₄ in a polar solvent, for example a mixture of water an ketone, in the presence of an amine such as N-methylmorpholine N-oxide. Alternative oxidation systems will be readily apparent to the person skilled in the are and can be found in standard references for organic synthesis such as Noyori, R. “Asymmetric catalysis in organic synthesis”, John Wiley and Sons, Inc. (1994) or Ojima, I. “Catalytic asymmetric synthesis VCH, (1993)

The dihydroxylated compound can be selectively protected. Indeed, when carrying out a protection such as with Cl-TBDMS we found that the hydroxyl at position 6 reacted until being completely protected, and only then the OH at position 5 is protected.

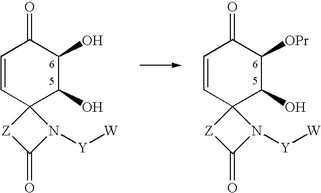

Without being bound by theory, we believe that this is due to the existence of C—H p interactions between the H at position 6 and the W group. This means that the —OH at position 6 is in an equatorial conformation, more reactive, while the —OH at position 5 is in an axial conformation, less reactive. This allows the selective reaction of one position with respect to the other.

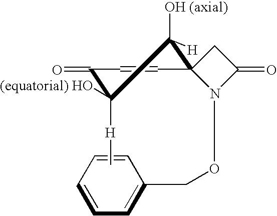

Therefore, both the facial selection (a versus β) when carrying out the hydroxylation, and the different reactivity of positions 5 and 6, due to the particular conformations generated by the presence of the W group and its interactions with the rest of the molecule, allows for a fine tuned control of the functionalisation of the molecule. This effect has been demonstrated by a very intensive NOE effect between the protons of the arylic portion and the protons of the cyclohexadiene moiety, and in particular with H at position 6.

In some cases the protecting group can migrate from position 6 to position 5 for example when the protecting group has migrating capacity such as with esters, carbonates, silylethers, etc. For example in the case of benzyl:

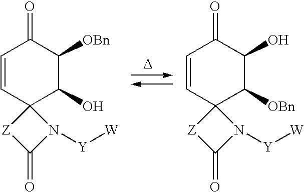

At the same time there is a change is the conformation as well, the OBn will be in equatorial position at the C5. In this way we can have one hydroxy or the other protected.

The two hydroxy groups can be protected with two protecting groups being the same as explained above, or with different protecting groups, first protecting the position 6 and then the position 5:

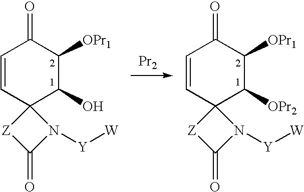

To introduce the hydroxyl protecting groups standard procedures can be used, such as those described in Greene and Wuts' “Protective Groups in Organic Synthesis”, John Wiley & Sons, Inc., New York, 1999 or Kocienski, P. J. “Protecting Groups”, 3^{rd} Ed. Thieme Chemistry, 2003.

In another embodiment the carbonyl group can also be selectively functionalized for example by Nucleophilic addition. Importantly, the lactam group does not react instead because it has a Weinreb type of amide. Thus cyanides, organolithium compounds, Grignard's reagents, azides, halogens, and ketones among other can be easily added to introduce the desired functionality at this position. If an hydride is used then an hydroxy at position 3 is generated. Suitable procedures for this kind of reactions are known in the art and can be found for example in Fischer, A. et al J. Org. Chem., 1987, 52, 4464-4468 “Formation of 4-nitrocyclohexa-2,5-dienols by addition of organolithium reagents to 4-alkyl-4-nitrocyclohexa-2,5-dione”; Wipf et al., Angew. Chem. Int. Ed. Engl. 1997, 36, no. 7, 764-767; Fischer, A. et al., Tetrahedron lett., 1980, 21, 701-704; Carreno, M. et al., J. Org. Chem., 1997, 62, 26, 9128-9137, or March, J “Advanced Organic Chemistry” 5^{th} Ed. (2001).

The additions can be done independently of the functionalisation of the other positions. If no other hydroxy groups are present:

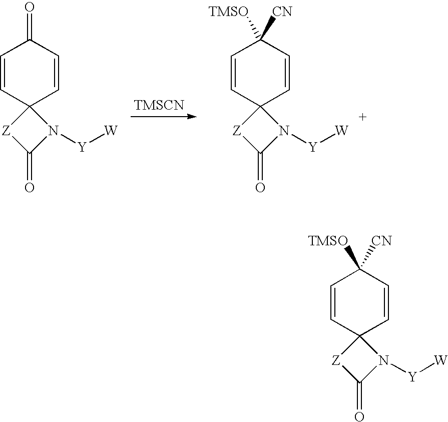

This reaction occurs with no stereoselectivity. The compounds can be in any case separated by resolution procedures know in the art, such as chromatography.

As an alternative to functionalisation, if desired the carbonyl group can be protected using know carbonyl protecting groups.

On the other hand, the additions can be done independently of the functionalisation of the other positions. We have found that when the compound of formula IV is first dihydroxylated and then the addition to the carbonyl is carried out, complete stereoselectivity is achieved. Although not completely clear, it appears that this important stereoselectivity is due to stereoelectronic effects between positions 6 and 7, and to the above mentioned conformation at position 6. For example:

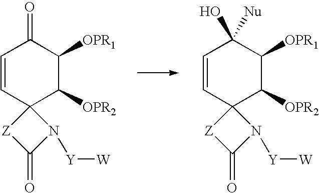

One particular example of such a process is the following:

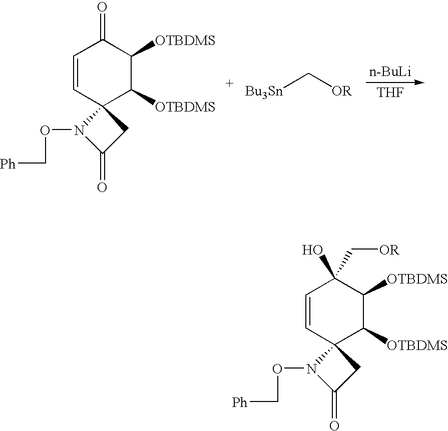

If for example PR₂CN is used, being PR a protecting group, the addition and the protection of the second hydroxyl group are carried out simultaneously.

Depending on the Nucleophilic reagent used the product of the reaction can have different structures. For example, if an epoxide with a defined conformation is desired, then a reagent such as an S ylide can be used. Under conditions of a Corey's epoxidation only one epoxide is obtained [a) E. J. Corey; Michael Chaykovsky J. Am. Chem. Soc. 87, 1965, 1353-1364. b) Steven P. Tanis, Mark C. McMills, Paul M. Herrinton J. Org. Chem. 50, 1985, 5887-5889. c) Malcolm Chandler, Richard Conroy, Anthony W. J. Cooper, R. Brian Lamont, Jan J. Scicinski, James E. Smart, Richard Storer, Niall G. Weir, Richard D. Wilson and Paul G. Wyatt J. Chem. Soc. Perkin Trans. 1, 1995, 1189-1197]:

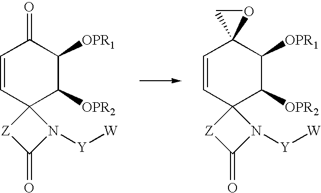

Alternatively a double bond can be generated, for example under Wittig's conditions. In this case the Nucleophile is a C ylide.

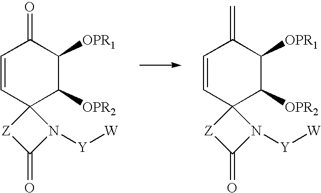

The double bond can have further substituents at position 10 depending on the reagent used. From this structure, with two differentiated double bonds, further reactions can be carried out as we will explain below.

If the Nucleophile is directed to the double bond instead of the carbonyl then the functionality or the additional carbon group is added at position 9:

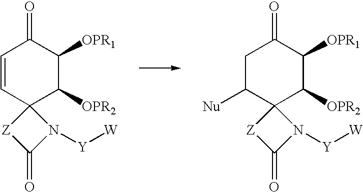

In such a way, groups such as cyanide, halogen or an allylic group can for example be introduced at position 9, opening new possibilities for further construction. It will be understood that once this Nu is introduced, then the above reactions on the carbonyl group, such as another nucleophilic attack can be done to generate new structures. For example, if Corey's epoxidation or Wittig's reaction are carried out on this new substrate, the following structures can be obtained:

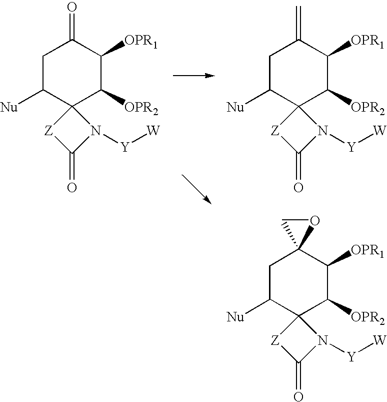

The epoxides can be opened to give an hydroxyl group at position 10. This can be done with the simultaneous introduction of a Nucleophilic or halogen group at position 9:

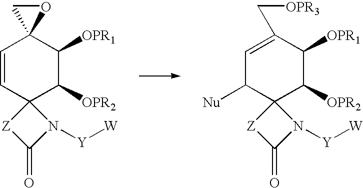

In this way we generate a new double bond between positions 7 and 8 which again opens many possibilities for further functionalisation. For example, a simple dihydroxylation will generate a highly functionalised structure, with two new and differentiated hydroxy groups:

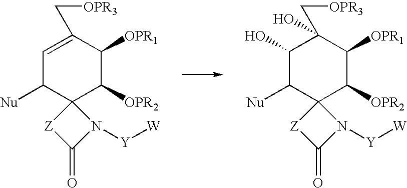

A further example of a combined protection and introduction of halogen following these ideas is:

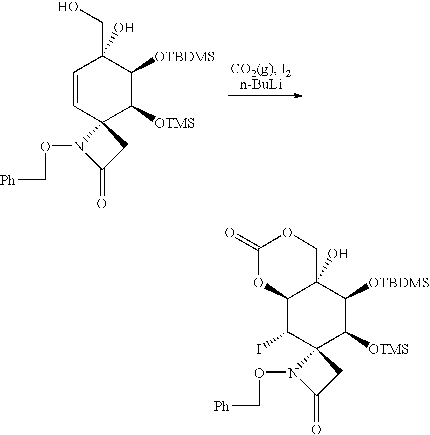

The halogen can then be easily exchanged with another group:

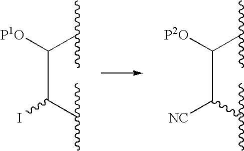

In another aspect of the invention, the second double bond (positions 4 and 5) can also be stereoselectively hydroxylated. This occurs more readily when the carbonyl group at position 3 is present, we think because it allows the in situ generation of an allylic alcohol, which might indicate that it plays a role in the oxidation process. Thus, under mild conditions:

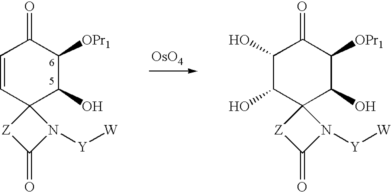

In this case the hydroxyl groups appear at the B face, we believe for stereoelectronic reasons. If a different stereochemistry is desired the appropriate oxidation or epimerization conditions can be selected. For example, under selective acidic or basic conditions the hydroxy at position 4 epimerizes. Alternatively hydroxyl inversions via the Mitsunobu type reaction, such as using DEAD, Ph₃P and an acid such as benzoic or p-nitrobenzoic acid, can be used. Frequently, the inversion via Mitsunobu needs protection of the other hydroxyl groups. Further details on the inversion via the Mitsunobu reaction can be found in Mitsunobu, O., Synthesis, 1, 1981; or Hughes, D. L., Org Reactions, 1992, 42, 335.

The dihydroxylation can be carried out on the product of Wittig's reaction, in this case two differentiated hydroxy groups are introduced at positions 7 and 10:

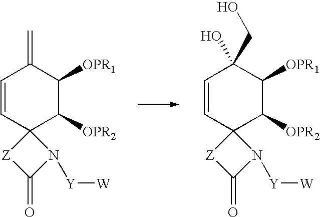

If instead of a dihydroxylation we do an epoxidation with, for example, a peroxide under mild conditions, we obtain an epoxide with just the opposite configuration at 7 when compared with the epoxide obtained directly under Corey's conditions:

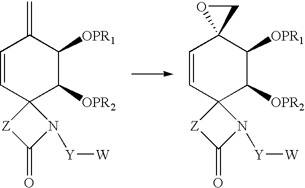

Thus, even through all these reactions we are still able to control the stereochemistry at the different positions. This allows us to prepare intermediates with the adequate configuration in order to reach downstream the different stereoisomers of biologically active compounds.

A further example of oxidation reaction is the epoxidation of the double bond between positions 8 and 9. Depending on the oxidation system used and the protecting groups present the epoxidation can give one configuration or another:

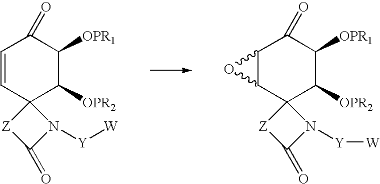

Below are some examples of this kind of reaction:

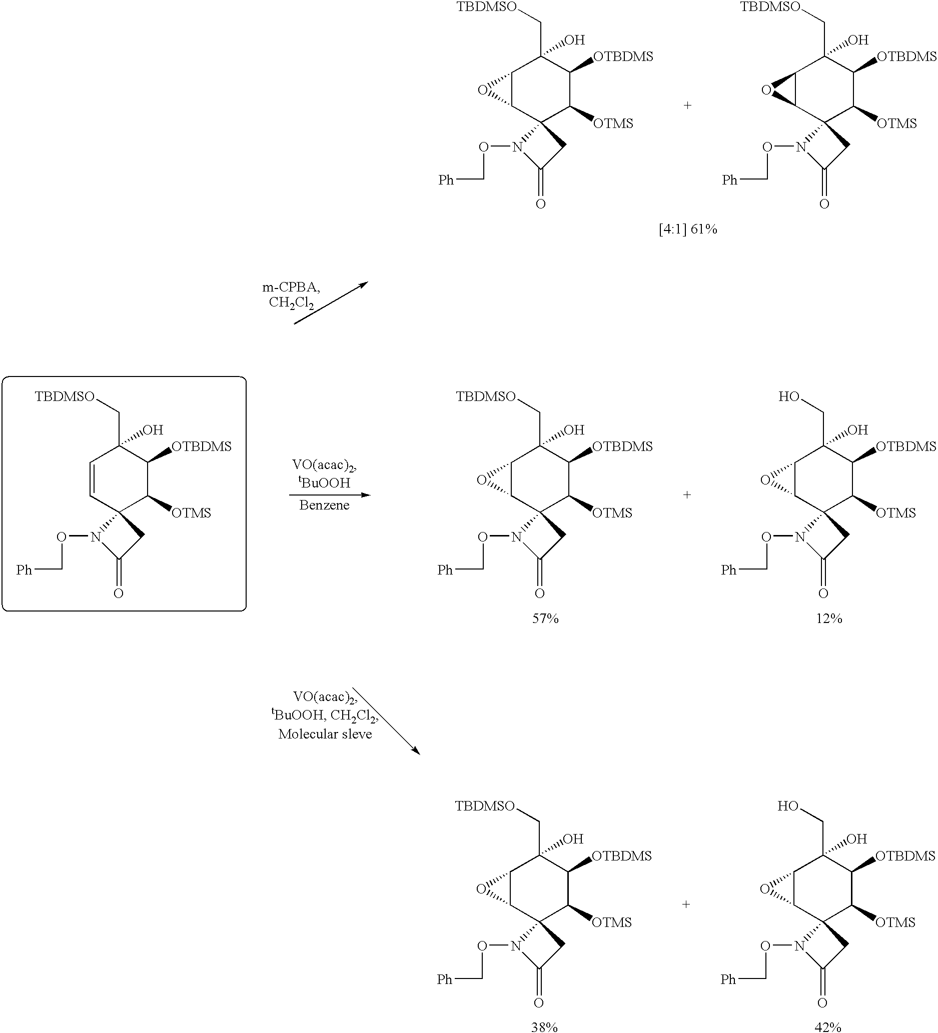

Further, these compounds can be selectively deprotected, the protecting groups exchanged or the epoxide can be opened with a Nucleophilic group or with an halogen. For example:

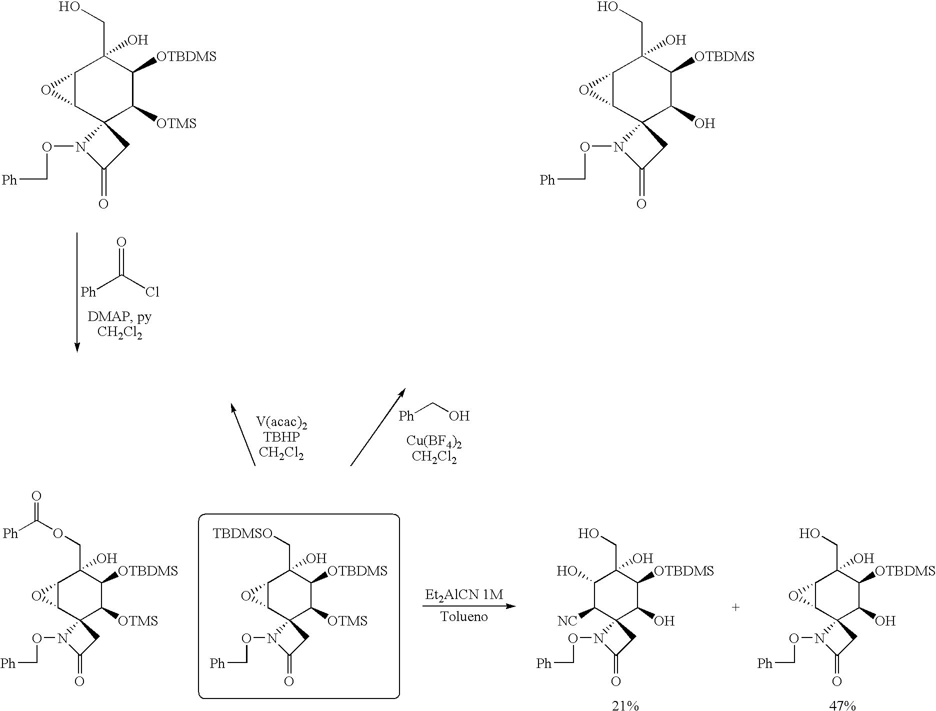

Of course, from the starting compound of formula IV a double epoxidation can take place:

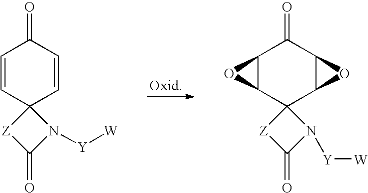

When carrying out oxidations, if the carbonyl group has already been functionalized, then stronger oxidation conditions are needed, such as the use of RuCl₃ or similar systems:

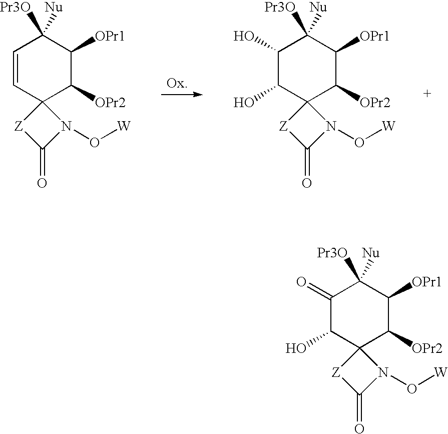

In this case depending on the amount of oxidating agent used or conditions of the reaction an overoxidation can occur, leading to the creation of a new carbonyl at position 8, in good yields, which opens the way to new possibilities. The carbonyl is generated at this position because is thermodynamically more stable. Both compounds are easily separated with well known procedures.

The conditions of dihydroxylation or ketohydroxylation can be controled. If less oxidating agent is used only the dihydroxycompound is obtained:

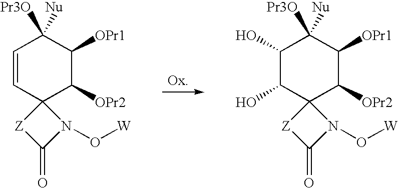

Further information on the reagents and conditions for the ketohydroxylation reactions can be found, for example, in Plietker, B. J. Org. Chem. 2003, 68, 7123-7125 and J. Org. Chem. xxx “The RuO₄-catalyzed ketohydroxylation. Part 1. Development, Scope and Limitation” whch are incorporated herein by reference in their entirety.

If a carbon (10) is present at position 7 then the carbonyl can be generated at position 9 instead. The balance of products obtained will be probably due to the en-diol equilibrium that gives rise to the thermodinamically more stable compounds:

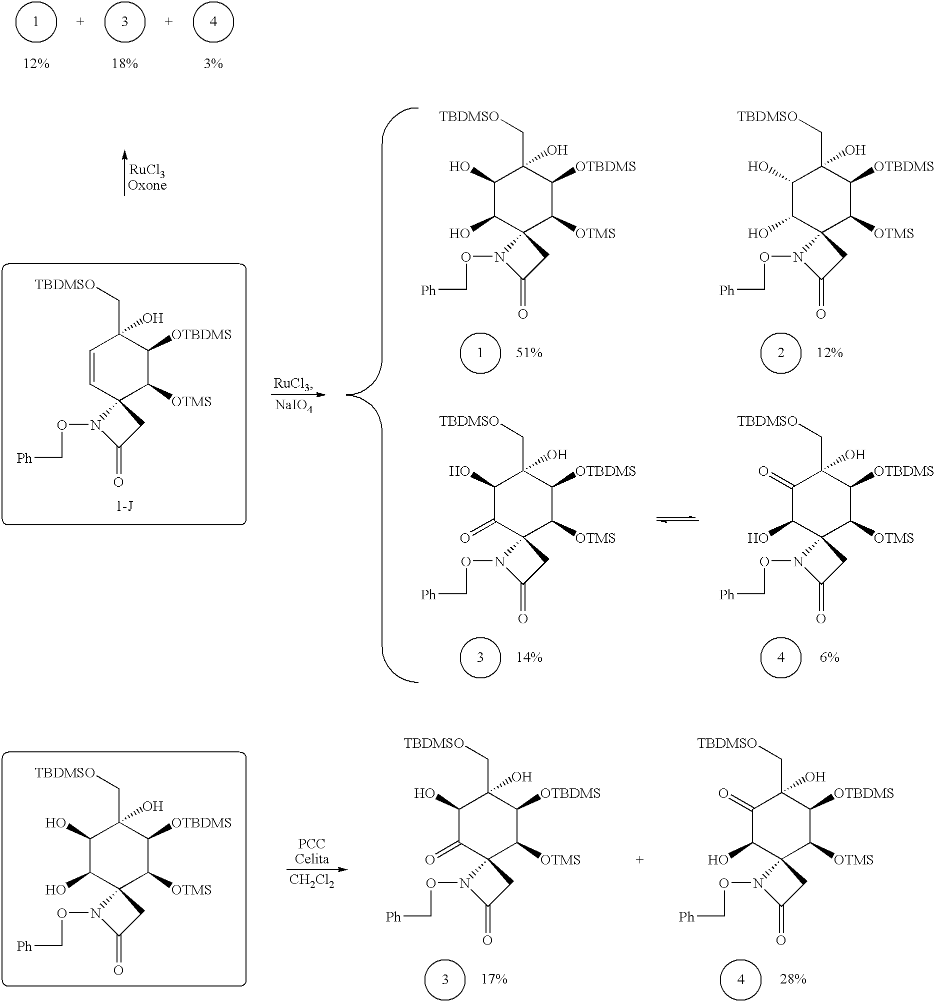

These compounds are easily separated by conventional procedures. Through this processes we introduce the carbonyl group at positions 8 or 9 as an altenative to position 7.

Complete orthogonal and complete regioselective protection can be achieved from here by introduction of a further protecting group:

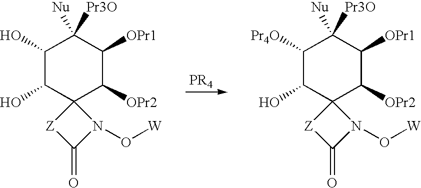

The presence of vicinal hydroxyl groups allows the simultaneous protection of two of them through the use of diol protecting groups if desired. Among the diol protecting groups that can be used we have O,O-acetals such as isopropylidene acetals (acetonides); cyclohexylidene and cyclopentylidene acetals; arylmethylene acetals; methylene acetals; diphenylmethylene acetals; 1,2-diacetals such as dispiroketal (dispoke) derivatives, cyclohexane-1,2-diacetals, butane-2,3-diacetals; silylene derivatives; 1,1,3,3-tetraisopropyldisiloxanylidene derivatives or N,O-acetals. Additional examples of diol protecting groups can be found in reference books such as Greene and Wuts' “Protective Groups in Organic Synthesis”, John Wiley & Sons, Inc., New York, 1999. Additionally, borolanes can be formed on the two vicinal hydroxy groups, for example using phenylboric acid.

One example of such a compound with vicinal protection is:

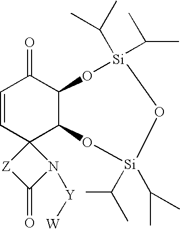

In this case the 7 member ring accommodates the axial-equatorial position of the two OH being protected.

As an alternative, dihydroxylation and protection can be carried out simultaneously:

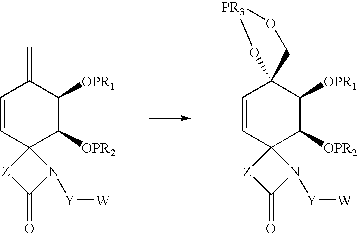

Or, if desired, only one of the hydroxy groups can be selectively protected due to their different nature (primary versus tertiary alcohol):

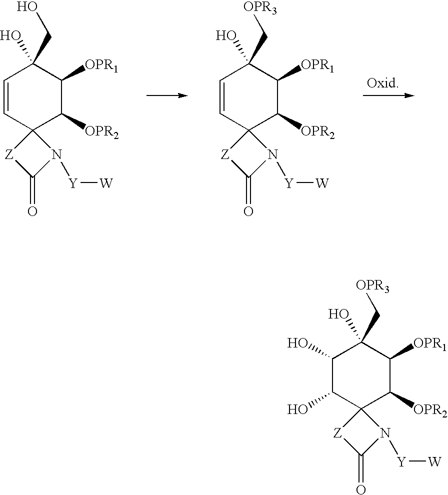

Depending on the protecting group used the stereochemistry of the second dihydroxylation can be controlled. For example:

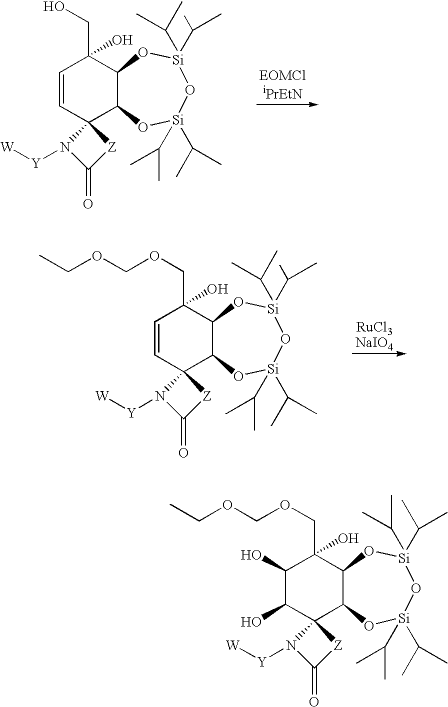

Thus to avoid that the second dihydroxylation or ketohydroxylation depends on thermodynamical factors, stereoselectivity can be controlled chemically instead using the appropriate protecting groups. Conformation plays an important role in these reactions. For example:

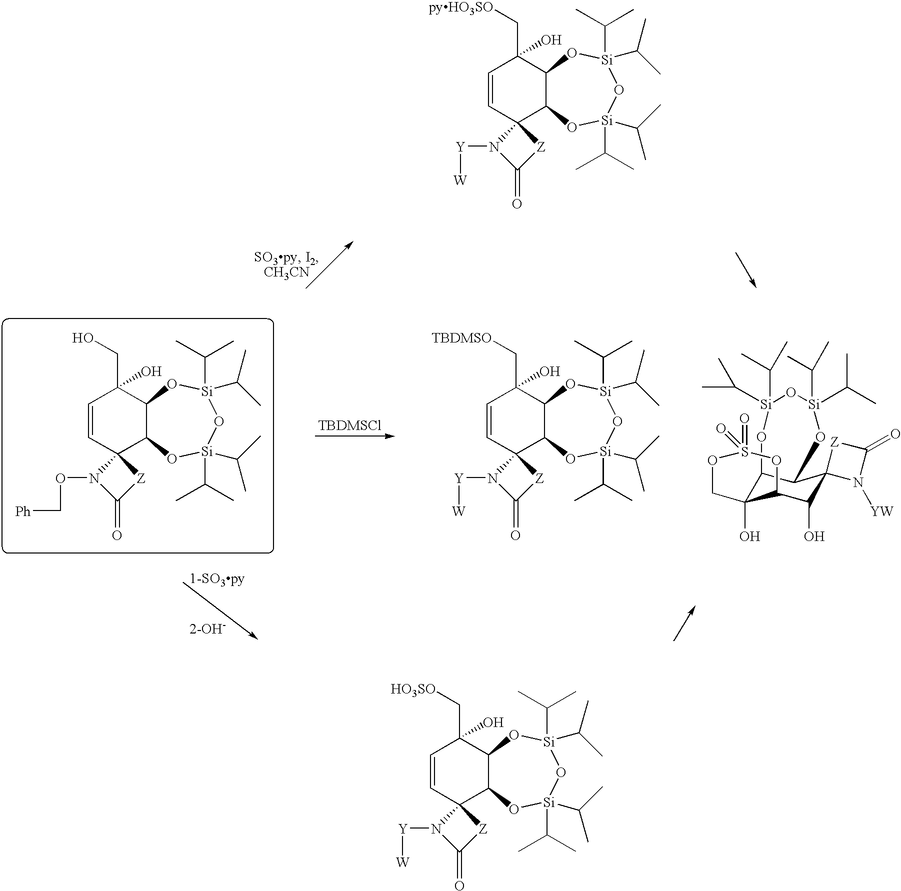

The use of protecting groups of a certain size, or that are linked simultaneously to two different positions can therefore control the stereochemistry of the product.

Further, a carbolactone can be produced through reaction of a functionalised Nucleophile at the carbonyl group and lactonization with the unprotected hydroxyl group which is present at position 8, for example using methyl (triphenylphosphoranylidene)acetate:

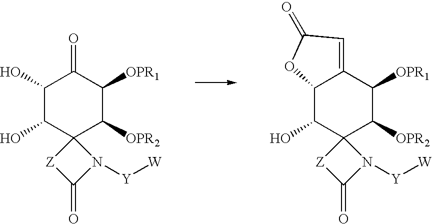

Or, under stronger conditions:

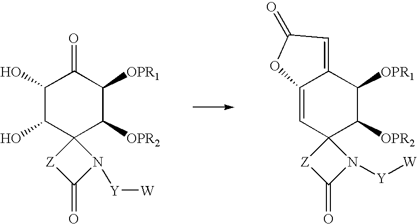

Again, we obtain a new structure plenty of functionalities with can be further subjected to additional reactions.

An alternative system of protection involves the formation of an orthoester. This provides for a very precise conformational control and the simultaneous protection of 3 hydroxy groups. Under adequate conditions the orthoester is formed starting form an ester protected hydroxy group. Further information on the conditions needed can be found for example in Giner J L et al. J. Org. Chem. 2002, 67:2717-2720 “A biomimetic approach to the synthesis of an antiviral marine steroidal orthoester” and references therein, which is incorporated by reference in its entirety. Thus structures of the following type, stabilized through an hydrogen bond, are easily accessible:

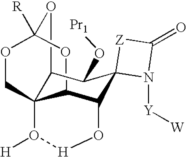

An example of a route to these compounds is:

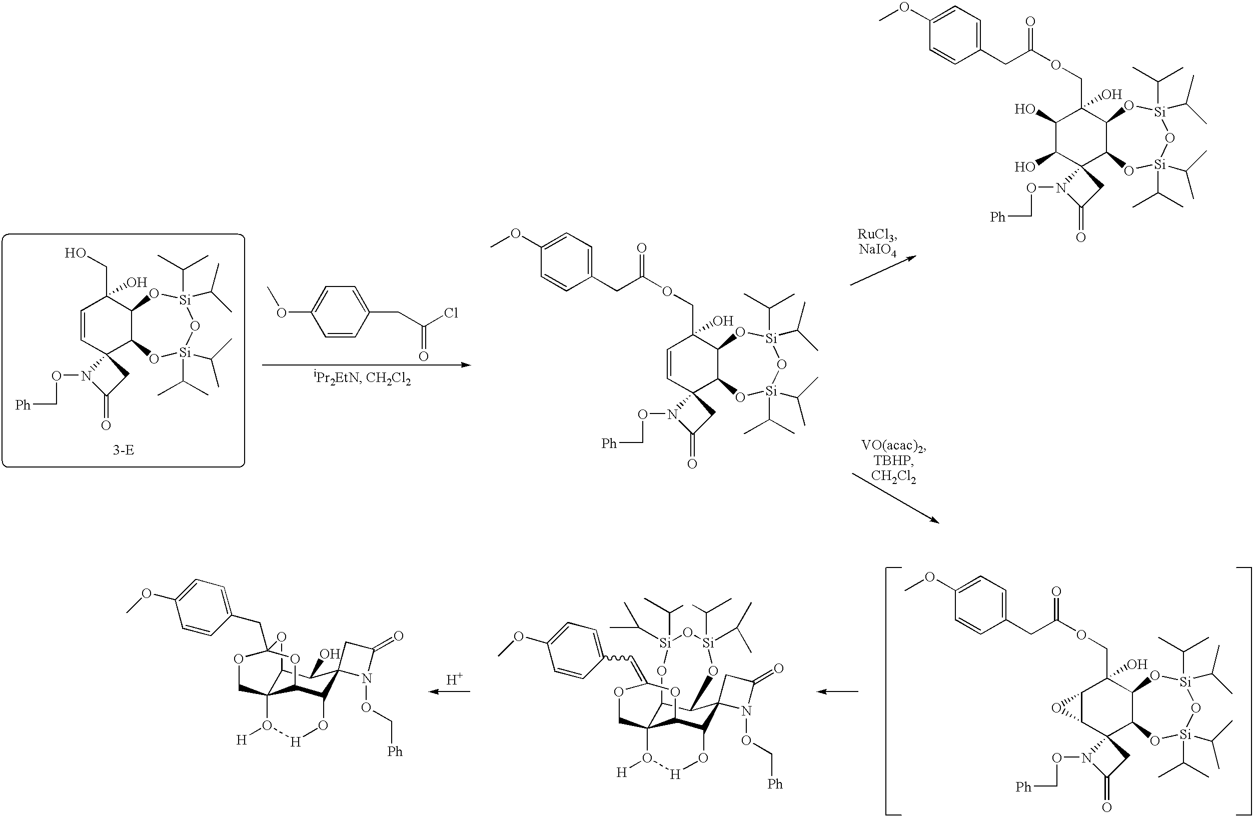

As we already mentioned, allylic rearrangements can be advantageously used to shift and introduce functionalities at the desired positions, for example following the scheme:

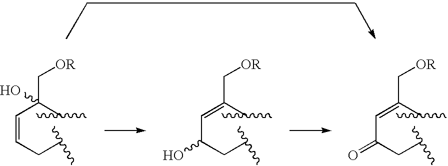

If the hydroxyl group is protected, then under appropriate conditions the —CH₂OR group or any other at this position can migrate instead.

This strategy provides stereospecific access to structures of the type:

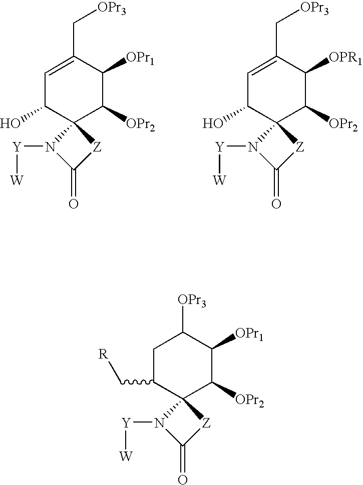

Example of such rearrangements are the following:

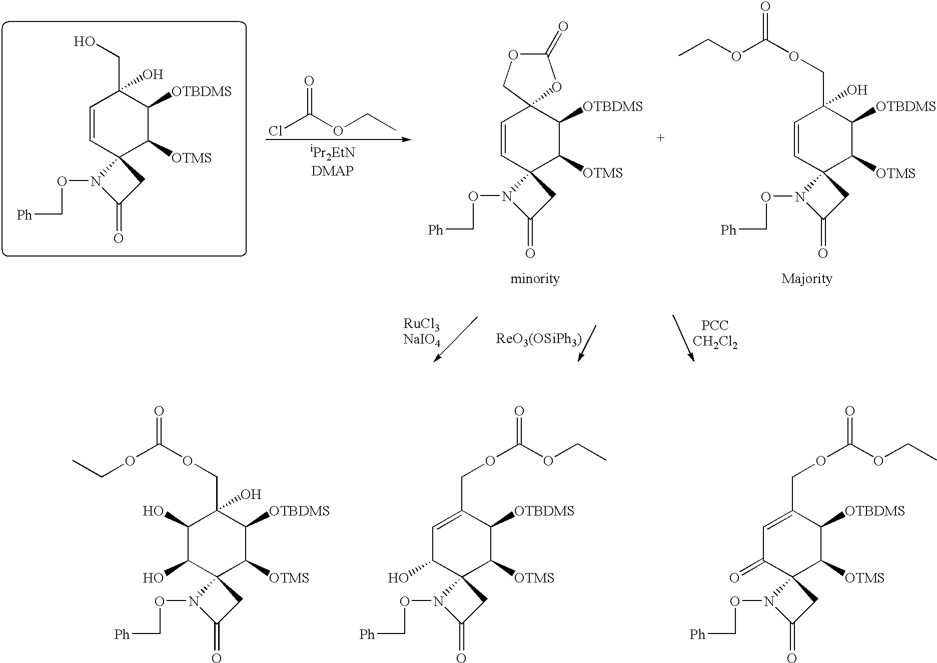

The following scheme shows more examples of the possibilities open using the basic set of reactions already described, with stereospecific control of the groups introduced. In this case Z is —CH₂—, but it can be any of the above defined, in particular Z can have a chiral centre. In this case enantiospecific reactions will be possible.

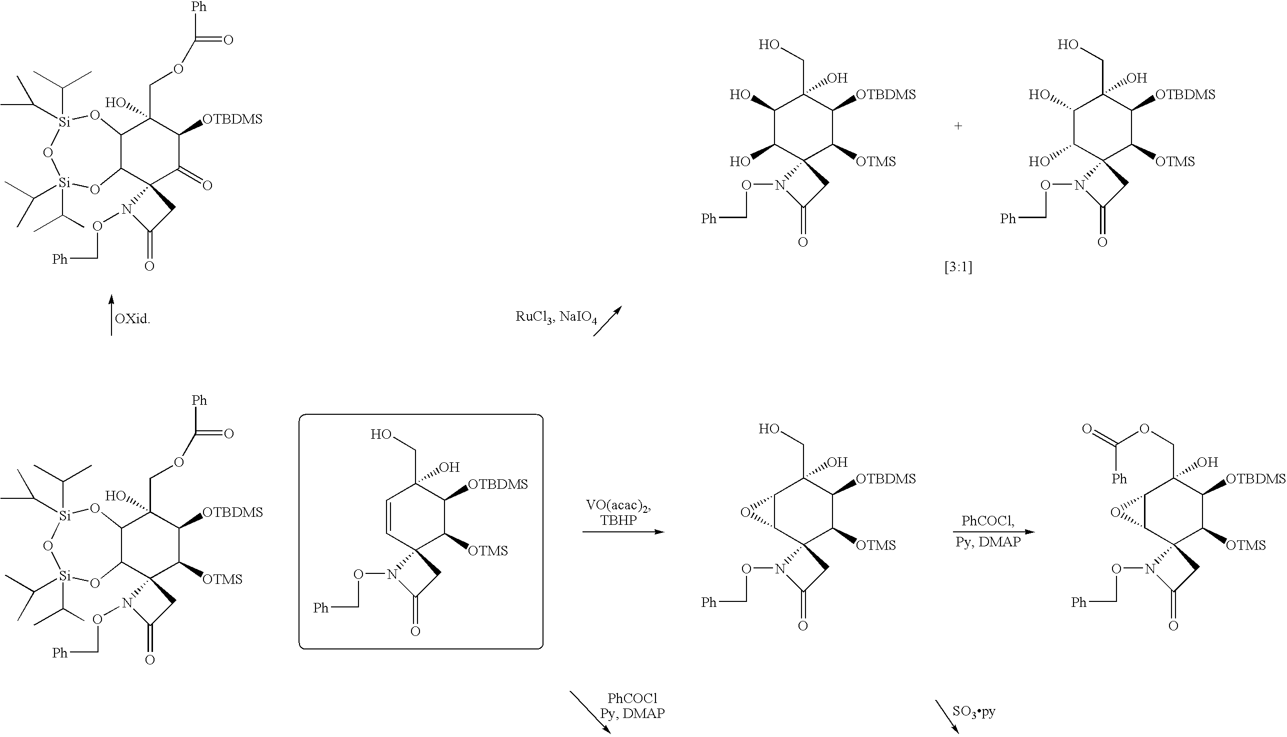

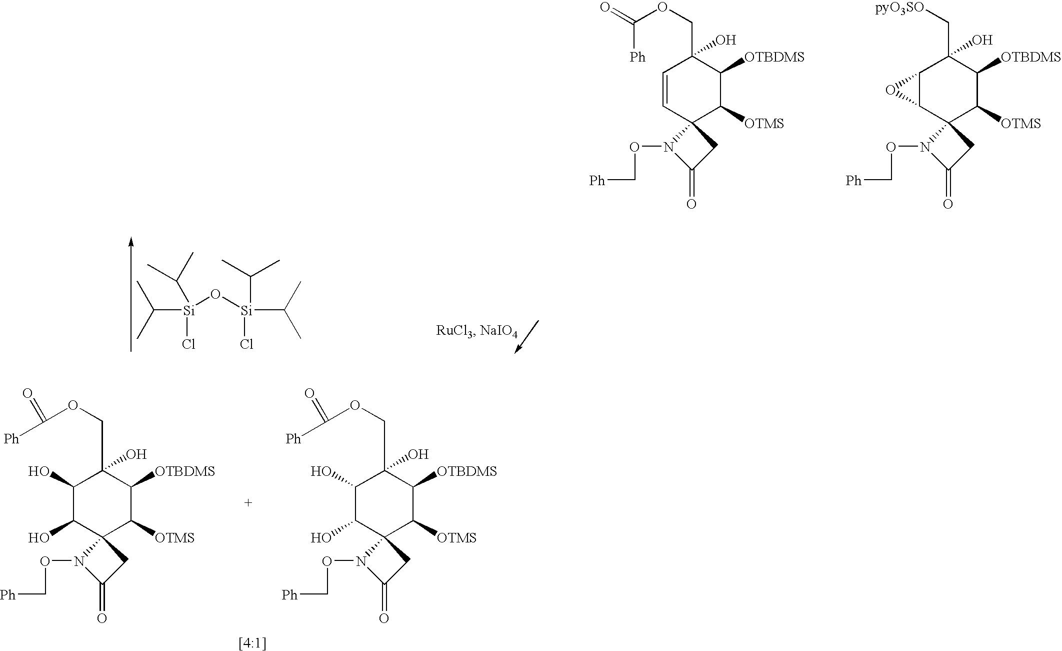

In one of these reactions, the protected hydroxyl group at position 5 is in an equatorial position and can be easily deprotected. Thus this position becomes accessible for further reactions. For example:

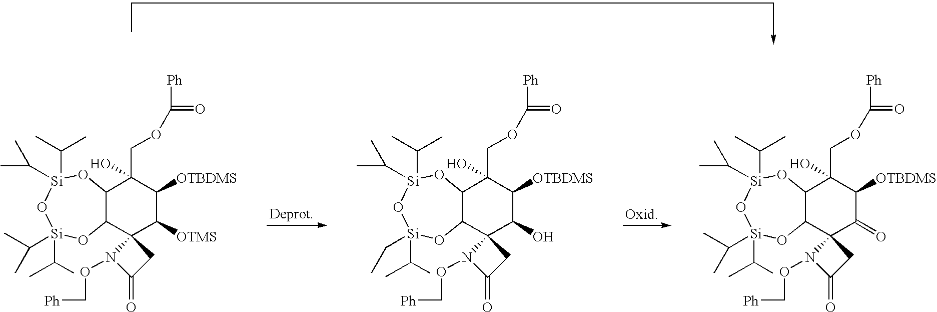

Thus a carbonyl group can also be introduced at position 5.

It will be readily apparent to the person skilled in the art that the compounds of the invention are suitable precursor to inositols and conduritols, through opening of the lactame ring and removing, later on, their acetate portion by processes that involve retroaldolic or retro-Staudinger like reactions.

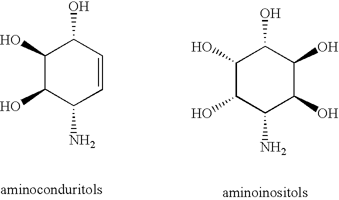

They are also suitable precursors to important natural products such as Pancrastistin, for example following a synthetic procedure as proposed below:

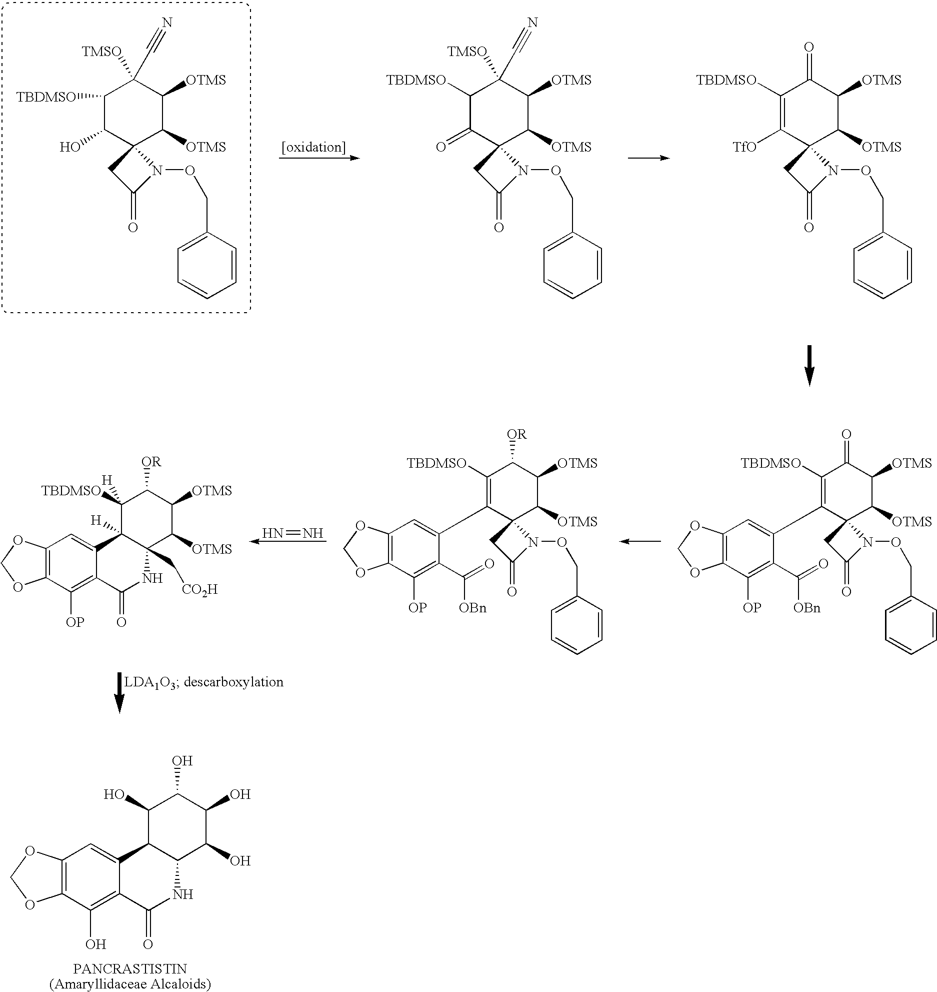

Other analogues of these valuable biological compounds can be prepared if we start from the different structures as we are describing here.

As can be understood from the above and will be apparent to the person skilled in the art, due to the particular conformation and reactivity characteristics of the described compounds, a great number of possibilities can be achieved. It is important to point out that the obtained compounds of formula I will present a carefully crafted functionality, in particular at the different positions 5, 6, 7, 8, 9 and 10, and the desired stereochemistry. The same structure can be reached through different combinations of the basic reactions. The introduction of different protecting groups opens the route to very selective further reactions by choosing the appropriate protection-deprotection strategies.

The process to obtain any of these compounds can be readily designed by starting from a compound of formula IV above and then applying a basic set of reactions selected from:
a) Hydroxilation, dihydroxylation or ketohydroxylation: as above explained, using mild (such as Os O₄/N oxide amine) or strong systems (such as RuCl₃) depending on the position to be hydroxylated. Alternative systems are also envisaged.
b) Nucleophilic attack at the carbonyl group: for example with a carbanion on a sp, sp2 or sp3 C, the carbanion can be prepared or generated in situ; or with an hydride. The Nucleophilic attack allows the introduction of new functionalities, new carbonated structures and also epoxides or double bonds, depending on the reagents used.
c) Hydroxyl inversion: for example through epimerization or inversion, for example in Mitsunobu conditions.
d) Hydroxyl or carbonyl protection: as explained above, using the same or different protecting groups in conditions as explained above.
e) Allylic rearrangements: allows migration of a double bond.

Although each of these procedures is well known and the appropriate reagents can be selected by the person skilled in the art of organic synthesis, for example from those given in the references above, their application to the our structures gives unexpected results in terms of reactivity and selectivity.

All the compounds will be obtained as racemic mixtures. However, if enantiopurity is desired, this can be achieved by introducing a chiral center in the Z and/or W groups as explained above, or using chiral reagents or catalysts. Therefore the compounds of the present invention represented by the above described formulae may include racemic mixtures, pure enantiomers or variable mixtures thereof depending on the presence of stereogenic centers or diastereoisomers. The single isomers, enantiomers or diastereoisomers and mixtures thereof fall within the scope of the present invention. Mixtures of different diasteroisomers can be separated by conventional techniques.

Unless otherwise stated, the compounds of the invention are also meant to include compounds which differ only in the presence of one or more isotopically enriched atoms.

For example, compounds having the present structures except for the replacement of a hydrogen by a deuterium or tritium, or the replacement of a carbon by a ¹³C— or ¹⁴C-enriched carbon or ¹⁵N-enriched nitrogen are within the scope of this invention.

The compounds of the invention may be in crystalline form either as free compounds or as solvates (e.g. hydrates) and it is intended that both forms are within the scope of the present invention. Methods of solvation are generally known within the art.

The invention will be further illustrated by means of examples, which should not be interpreted as limiting the scope of the claims.

### EXAMPLES

### General Methods and Materials

All reactions described below were carried out under argon atmosphere unless otherwise noted. The solvents used were distilled and dried under argon atmosphere before use. All starting materials were purchased commercially (Aldrich, Fluka and Merck) and used without further purification. Flash Chromatography was executed on columns loaded with 230-400 mesh silica gel Merck. TLC was carried out on silica gel Merck (Kieselgel 60F-254).

Melting points (mp) were determined on a Reichert Microscopic Hot-Stage and are uncorrected. ¹H and ¹³C NMR spectra were measured on a Varian Gemini-200 and a Varian Inova-300 spectrometer with (CH₃)₄Si as an internal reference and CDCl₃ as solvent unless otherwise noted. Both ¹H and ¹³C NMR spectral data are reported in parts per million (δ) relative to residual sign of the solvent (CDCl₃, 7.26 ppm and 77.0 ppm for ¹H and ¹³C NMR, respectively). ¹H and ¹³C NMR designations are: s (singlete); s br. (broad singlete); d (doublete); t (triplete); q (quartete); m (multiplete). Infrared (IR) spectra were record on a Perkin-Elmer FT-IR spectrometer. UV spectra were record on a Perkin-Elmer 402 spectrometer. Low-resolution mass (LRMS) spectra were obtained on a Hewlett Packard 5973 MSD spectrometer with a direct inlet system (EI) at 70 eV. Microanalytical data (E.A.) were obtained on a Perkin-Elmer 240C and Heraus CHN—O instruments at the Instrumental Analysis Department of Instituto de Química Orgánica General (C.S.I.C.).

The compounds below with Z O—CH₂— are nominated as derivatives of 1-azaspiro[3.5]nonan-2-one and numerated following the numeration described below.

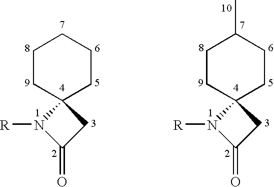

### Example 1

### Preparation of rac-(4R,5S,6S)-1-benzyloxy-5,6-dihydroxy-1-azaspiro[3.5]nona-8-ene-2,7-dione (2)

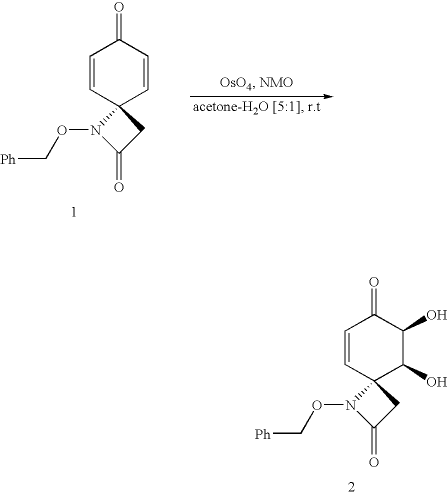

To a solution of meso-1-benzyloxy-1-azaspiro[3.5]nona-5,8-diene-2,7-dione (1) (804 mg, 3.150 mmol) in acetone (12 ml) was added sequentially at room temperature H₂O (2.4 ml), N-methylmorpholine N-oxide (812 mg, 6.930 mmol) and osmium tetroxide (2.37 ml, 2.5 wt. % solution in 2-methyl-2-propanol, 0.189 mmol). The resulting mixture was stirred at room temperature until the reaction was complete (1 h, TLC monitoring, AcOEt), and then quenched with 10% aqueous Na₂S₂O₃ solution (3 ml). After 20 min, the mixture was extracted with AcOEt (5×6 ml). The combined organic extracts were dried over Na₂SO₄, filtered and concentrated under reduced pressure. The residue was purified by silica gel column chromatography (hexane-AcOEt, 3:2) to give rac-(4R,5S,6S)-1-benzyloxy-5,6-dihydroxy-1-azaspiro[3.5]nona-8-ene-2,7-dione (2) as a white solid (419 mg, 46%).

Rf=0.47 (TLC, AcOEt); yield, 46%; white solid; ¹H-NMR (200 MHz, CD₃OD): δ 7.56 (5H, s, Ph), 6.69 (1H, part A syst. AB, J_{9,8}=10.1 Hz, H-9), 6.65 (1H, part B syst. AB, J_{8,9}=10.1 Hz, H-8), 5.19 (1H, part A syst. AB, J=11.2 Hz, OCH₂Ph), 5.14 (1H, part B syst. AB, J=11.2 Hz, OCH₂Ph), 4.56 (1H, d, J=2.9 Hz, H-6), 4.31 (1H, m, H-5), 3.32 (1H, part A syst. AB, J=14.4 Hz, H-3), 2.93 (1H, part B syst. AB, J=14.4, H-3); ¹³C-NMR (75 MHz, CD₃OD): δ 199.0, 167.0, 146.8, 136.7, 131.2, 131.1, 130.1, 80.9, 76.1, 74.2, 67.3, 43.4; IR (KBr): ν 3429, 1772, 1692, 1631, 1450, 1382, 1053, 698 cm⁻¹; LRMS (API-ES⁺): m/z 312 (M+Na)⁺, 290 (M+H)⁺.

### Example 2

### Preparation of rac-(4R,5S,6S)-1-benzyloxy-6-tert-butyldimethylsilyloxy-5-hydroxy-1-azaspiro[3.5]nona-8-ene-2,7-dione (3)

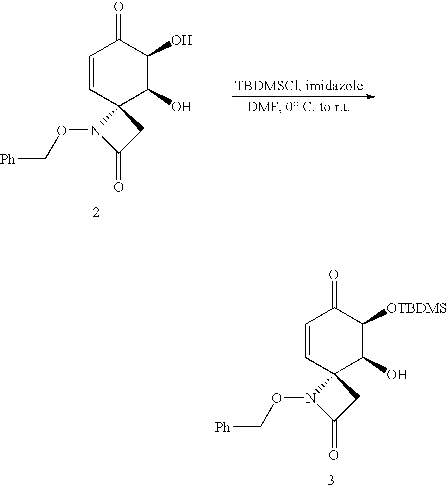

To a solution of rac-(4R,5S,6S)-1-benzyloxy-5,6-dihydroxy-1-azaspiro[3.5]nona-8-ene-2,7-dione (2) (123 mg, 0.425 mmol) and imidazole (35 mg, 0.510 mmol) in DMF (0.6 ml) was added at 0° C. a solution of tert-butyldimethylsilyl chloride (77 mg, 0.510 mmol) in DMF (1.2 ml). After 12 h at room temperature, the reaction was quenched with H₂O (3 ml) and the mixture extracted with AcOEt (3×5 ml). The combined extracts were washed with saturated aqueous CuSO₄ solution (2×10 ml) and brine (2×10 ml), dried over Na₂SO₄, filtered and concentrated under reduced pressure. The residue was purified by silica gel column chromatography (hexane-AcOEt, 5:2) to give rac-(4R,5S,6S)-1-benzyloxy-6-tert-butyldimethylsilyloxy-5-hydroxy-1-azaspiro[3.5]nona-8-ene-2,7-dione (3) as a white solid (133 mg, 78%).

R_{f}=0.56 (TLC, hexane-AcOEt, 1:1); yield, 78%; white solid; ¹H-NMR (200 MHz, CDCl₃): δ 7.40-7.29 (5H, m, Ph), 6.24 (1H, part A syst. AB, J_{9,8}=10.1 Hz, H-9), 5.78 (1H, part B syst. AB, J_{8,9=}10.1 Hz, H-8), 5.00 (1H, part A syst. AB, J=11.4 Hz, OCH₂Ph), 4.87 (1H, part B syst. AB, J=11.4 Hz, OCH₂Ph), 4.36 (1H, d, J=2.7 Hz, H-6), 4.01 (1H, m, H-5), 3.25 (1H, part A syst. AB, J=14.6 Hz, CH₂), 2.63 (1H, part B syst. AB, J=14.6 Hz, CH₂), 2.56 (1H, d, J=3.8 Hz, OH), 0.85 (9H, s, C(CH₃)₃), 0.09 (3H, s, SiCH₃), 0.08 (3H, s, SiCH₃); ¹³C-NMR (75 MHz, CDCl₃): δ 195.4, 165.5, 145.2, 134.9, 129.6, 129.5, 129.3, 128.8, 79.3, 75.5, 71.7, 64.7, 41.9, 25.6, 18.1, −4.9, −5.3; IR (KBr): ν 3453, 2949, 2929, 2855, 1767, 1682, 1256, 1119, 1088, 980, 843, 782 cm⁻¹; LRMS (API-ES⁺): m/z 829 (2M+Na)⁺, 426 (M+Na)⁺, 404 (M+H)⁺.

The structure of compound 3 and relative configuration was confirmed using a MARRESEARCH mar345 difractometer with an Image plate detector. The X-Ray crystal data and atomic coordinates are as follows:

Crystal data

| Empirical formula | C₂₁H₂₉NO₅Si |
|---|---|
| Formula weight | 403.54 |
| Temperature | 293(2) K |
| Wavelength (Å) | 0.71073 |
| Crystal system, space group | Monoclinic, P21/c |
| Unit cell dimensions | a = 24.9570(10) Å |
| | b = 7.8220(10) (7) |
| | c = 11.5760(10) |
| | β = 92.2070(10)° |
| Volume | 2258.1(4) Å³ |
| Z, Calculated density | 4, 1.187 Mg/m³ |
| μ (mm⁻¹) | 0.133 |
| F(000) | 864 |

Atomic coordinates (×10⁴) and equivalent isotropic displacement
parameters (Å² × 10³). U(eq) is defined as one third of the trace
of the orthogonalized Uᵢⱼ tensor.

| | x | y | z | U(eq) |
|---|---|---|---|---|
| Si | 1354(1) | 4300(2) | 3824(2) | 77(1) |
| O(1) | 3942(2) | 7425(5) | 3388(3) | 78(1) |
| O(2) | 3264(2) | 10511(5) | 3803(4) | 81(1) |
| O(3) | 2772(2) | 5453(4) | 5102(3) | 83(1) |
| O(4) | 1944(1) | 4772(4) | 3271(3) | 71(1) |
| O(5) | 2436(2) | 1848(5) | 1822(4) | 95(1) |
| N | 3404(2) | 7585(5) | 3627(4) | 71(1) |
| C(1) | 5028(3) | 5810(7) | 3142(6) | 77(2) |
| C(2) | 5543(3) | 5917(10) | 2872(7) | 104(2) |
| C(3) | 5882(3) | 6929(10) | 3532(9) | 111(3) |
| C(4) | 5703(4) | 7864(9) | 4425(8) | 104(2) |
| C(5) | 5167(3) | 7764(8) | 4672(6) | 89(2) |
| C(6) | 4819(2) | 6762(7) | 4062(6) | 72(2) |
| C(7) | 4241(3) | 6634(10) | 4331(6) | 104(2) |
| C(8) | 3121(2) | 9067(7) | 3627(5) | 68(2) |
| C(9) | 2604(2) | 8103(6) | 3400(5) | 64(2) |
| C(10) | 2956(2) | 6507(6) | 3204(4) | 60(1) |
| C(11) | 2866(2) | 4907(6) | 3931(5) | 63(1) |
| C(12) | 2424(2) | 3796(6) | 3422(5) | 66(2) |
| C(13) | 2552(2) | 3246(9) | 2208(5) | 75(2) |
| C(14) | 2814(3) | 4538(8) | 1514(5) | 81(2) |
| C(15) | 2991(3) | 6005(8) | 1950(6) | 86(2) |
| C(16) | 1233(3) | 1961(8) | 3670(9) | 132(3) |
| C(17) | 1349(3) | 5018(12) | 5338(6) | 121(3) |
| C(18) | 862(2) | 5564(7) | 2940(6) | 83(2) |
| C(19) | 992(4) | 7483(9) | 3052(8) | 137(3) |
| C(20) | 289(3) | 5290(11) | 3361(8) | 131(3) |
| C(21) | 878(4) | 5033(13) | 1648(8) | 149(3) |

### Example 3

### Preparation of rac-(4R,5S,6S,8S,9S)-1-benzyloxy-6-tert-butyldimethylsilyloxy-5,8,9-trihydroxy-1-azaspiro[3.5]nona-2,7-dione (4)

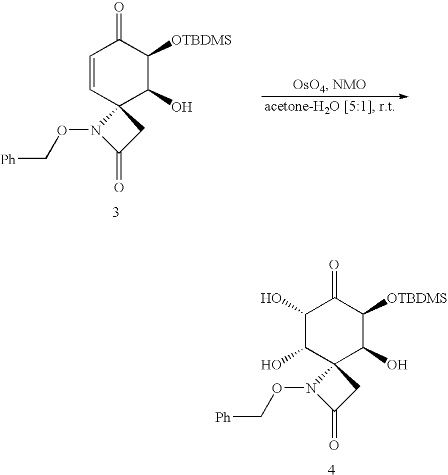

To a stirred solution of rac-(4R,5S,6S)-1-benzyloxy-6-tert-butyldimethylsilyloxy-5-hydroxy-1-azaspiro[3.5]nona-8-ene-2,7-dione (3) (50 mg, 0.124 mmol) in acetone (0.45 ml) was added sequentially at room temperature H₂O (0.09 ml), N-methylmorpholine N—oxide (58 mg, 0.496 mmol) and osmium tetroxide (93 μl, 2.5 wt. % solution in 2-methyl-2-propanol, 7.4·10⁻³ mmol). The resulting mixture was stirred at room temperature until the reaction was complete (18 h, TLC monitoring, hexane-AcOEt, 1:1), and then quenched with 10% aqueous Na₂S₂O₃ solution (20 drops). After 20 min, the mixture was filtered through silica gel with MeOH and concentrated under reduced pressure. The residue was purified by silica gel column chromatography (hexane-AcOEt, 2:1) to give rac-(4R,5S,6S,8S,9S)-1-benzyloxy-6-tert-butyldimethylsilyloxy-5,8,9-trihydroxy-1-azaspiro[3.5]nona-2,7-dione (4) as a white solid (26 mg, 48%).

R_{f}=0.46 (TLC, hexane-AcOEt, 1:1); yield, 48%; white solid; ¹H-NMR (200 MHz, CDCl₃): δ 7.49-7.30 (5H, m, Ph), 5.11 (1H, part A syst. AB, J=10.6 Hz, OCH₂Ph), 5.05 (1H, part B syst. AB, J=10.6 Hz, OCH₂Ph), 4.68 (1H, dd, J=4.3, 3.7 Hz, H-8), 4.44-4.32 (2H, m, H-5 and H-6), 4.22 (1H, dd, J=1.8, 1.5 Hz, H-9), 3.48 (1H, d, J=4.3 Hz, HO—C (8)), 3.36 (1H, part A syst. AB, J=14.3 Hz, CH₂), 2.73 (1H, d, J=1.8 Hz, HO—C (9)), 2.45 (1H, part B syst. AB, J=14.3 Hz, CH₂), 2.28 (1H, s br, HO—C (5)), 0.85 (9H, s, C(CH₃)₃), 0.13 (3H, s, SiCH₃), 0.04 (3H, s, SiCH₃); ¹³C-NMR (75 MHz, CDCl₃): δ 204.8, 166.1, 135.1, 129.4, 129.0, 128.5, 78.9, 76.4, 75.5, 72.2, 67.4, 65.7, 37.5, 25.6, 18.0, −5.0, −5.3; IR (KBr): ν 3435, 2949, 2927, 2855, 1761, 1740, 1631, 1261, 1110, 1078, 837 cm⁻¹; LRMS (API-ES⁺): m/z 897 (2M+Na)⁺, 460 (M+Na)⁺, 438 (M+H)⁺.

### Example 4

### Preparation of rac-(4R,5S,6S,8S,9S)-1-benzyloxy-6-tert-butyldimethylsilyloxy-5-hydroxy-8,9-dimethylmethylendioxy-1-azaspiro[3.5]nona-2,7-dione (5)

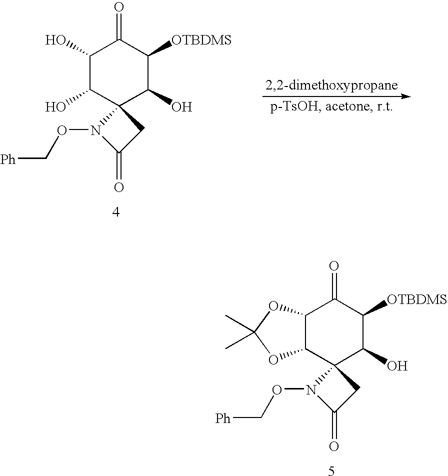

To a stirred solution of rac-(4R,5S,6S,8S,9S)-1-benzyloxy-6-tert-butyldimethylsilyloxy-5,8,9-trihydroxy-1-azaspiro[3.5]nona-2,7-dione (4) (50 mg, 0.115 mmol) and 2,2-dimethoxypropane (71 μl, 0.575 mmol) in dry acetone (0.5 ml) was added at room temperature catalytic amount of p-TsOH (1% mmol). The resulting mixture was stirred at room temperature for 18 h, then quenched with saturated aqueous Na₂CO₃ solution (1 ml) and extracted with AcOEt (3×2 ml). The combined organic extracts were washed with brine (3 ml), dried over Na₂SO₄, filtered and concentrated under reduced pressure. The residue was purified by silica gel column chromatography (hexane-AcOEt, 3:1) to give rac-(4R,5S,6S,8S,9S)-1-benzyloxy-6-tert-butyldimethylsilyloxy-5-hydroxy-8,9-dimethylmethylendioxy-1-azaspiro[3.5]nona-2,7-dione (5) as a white solid (27 mg, 49%) and unreacted starting material (5) (13 mg, 24%).

R_{f}=0.77 (TLC, hexane-AcOEt, 1:1); yield, 49%; white solid; ¹H-NMR (200 MHz, CDCl₃): δ 7.40-7.34 (5H, m, Ph), 5.05 (1H, part A syst. AB, J=10.2 Hz, OCH₂Ph), 5.02 (1H, part B syst. AB, J=10.2 Hz, OCH₂Ph), 4.84 (1H, d, J=5.4 Hz, H-8), 4.45 (1H, d, J=5.4 Hz, H-9), 4.39 (1H, d, J=2.7 Hz, H-6), 4.35 (1H, dd, J=5.9, 2.7 Hz, H-5), 3.45 (1H, part A syst. AB, J=14.2 Hz, CH₂), 2.56 (1H, d, J=5.9 Hz, OH), 2.48 (1H, part B syst. AB, J=14.2 Hz, CH₂), 0.86 (9H, s, C(CH₃)₃), 0.11 (3H, s, SiCH₃), 0.06 (3H, s, SiCH₃); ¹³C-NMR (75 MHz, CDCl₃): δ 203.6, 165.8, 135.0, 129.3, 128.9, 128.5, 111.3, 79.8, 79.0, 78.9, 77.7, 66.1, 65.4, 39.4, 26.9, 25.9, 25.5, 18.0, −5.1, −5.3; IR (KBr): ν 3434, 2930, 2855, 1764, 1739, 1628, 1453, 1384, 1255, 1225, 1111, 1086, 898, 832, 784 cm⁻¹; LRMS (API-ES⁺): m/z 977 (2M+Na)⁺, 500 (M+Na)⁺, 478 (M+H)⁺.

### Example 5

### Preparation of rac-(4R,5S,6S)-1-benzyloxy-6-tert-butyldimethylsilyloxy-5-trimethylsilyloxy-1-azaspiro[3.5]nona-8-ene-2,7-dione (6)

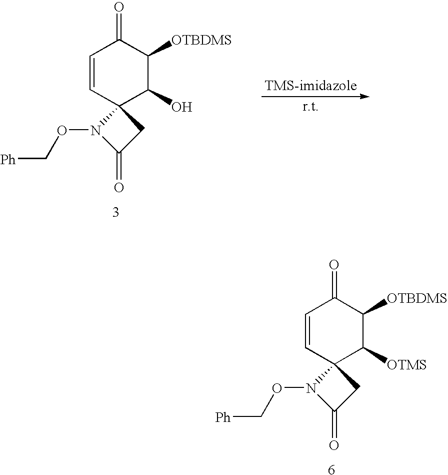

A solution of rac-(4R,5S,6S)-1-benzyloxy-6-tert-butyldimethylsilyloxy-5-hydroxy-1-azaspiro[3.5]nona-8-ene-2,7-dione (3) (130 mg, 0.322 mmol) in 1-trimethylsilylimidazole (0.5 ml, 3.220 mmol) was stirred at room temperature for 16 h. The reaction was quenched with Na₂PO₄ 0.1 M buffer (2 ml) and the mixture extracted with AcOEt (3×4 ml). The combined extracts were washed with saturated aqueous CuSO₄ solution (1×8 ml) and brine (2×8 ml), dried over Na₂SO₄, filtered and concentrated under reduced pressure. The residue was purified by silica gel column chromatography (hexane-AcOEt, 5:1) to give rac-(4R,5S,6S)-1-benzyloxy-6-tert-butyldimethylsilyloxy-5-trimethylsilyloxy-1-azaspiro[3.5]nona-8-ene-2,7-dione (6) as a white solid (100 mg, 65%).

R_{f}=0.50 (TLC, hexane-AcOEt, 3:1); yield, 65%; white solid; ¹H-NMR (200 MHz, CDCl₃): δ 7.42-7.28 (5H, m, Ph), 6.09 (1H, part A syst. AB, J_{9,8}=10.3 Hz, H-9), 5.68 (1H, part B syst. AB, J_{8,9}=10.3 Hz, H-8), 4.98 (1H, part A syst. AB, J=11.6 Hz, OCH₂Ph), 4.84 (1H, part B syst. AB, J=11.6 Hz, OCH₂Ph), 4.13 (2H, d, J=2.4 Hz, H-5 and H-6), 3.40 (1H, m, CH₂), 2.47 (1H, d, J=14.2 Hz, CH₂), 0.79 (9H, S, C(CH₃)₃), 0.16 (9H, s, Si(CH₃)₃), 0.08 (3H, s, SiCH₃), 0.00 (3H, s, SiCH₃); LRMS (API-ES⁺): m/z 973 (2M+Na)⁺, 498 (M+Na)⁺, 476 (M+H)⁺; LRMS (E1): m/z 475 (M⁺, 3), 460(2), 418(21), 384(5), 368(4), 354(6), 340(4), 324(3), 309(13), 269(43), 179(24), 147(34), 91(100), 73 (57).

### Example 6

### Preparation of rac-(4R,5S,6S,7S)-1-benzyloxy-6-tert-butyldimethylsilyloxy-7-cyano-5,7-bis(trimethylsilyloxy)-1-azaspiro[3.5]nona-8-ene-2-one (7).

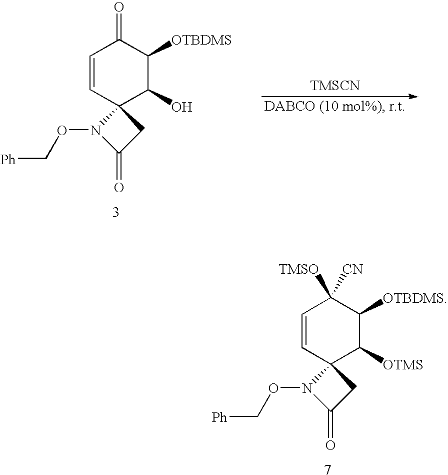

To a mixture of rac-(4R,5S,6S)-1-benzyloxy-6-tert-butyldimethylsilyloxy-1-azaspiro[3.5]nona-8-ene-2,7-dione (3) (200 mg, 0.496 mmol) and trimethylsilyl cyanide (607 μl, 4.460 mmol) at room temperature (cooled with water bath) was slowly added DABCO (6 mg, 0.050 mmol). The mixture was stirred at room temperature for 14 h and then concentrated under reduced pressure. The residue was purified by silica gel column chromatography (hexane-AcOEt, 5:1) to give rac-(4R,5S,6S,7S)-1-benzyloxy-6-tert-butyldimethylsilyloxy-7-cyano-5,7-bis(trimethylsilyloxy)-1-azaspiro[3.5]nona-8-ene-2one (7) as a orange solid (254 mg, 89%).

Rf=0.61 (TLC, hexane-AcOEt, 3:1); yield, 89%; orange solid; ¹H-NMR (300 MHz, CDCl₃): δ 7.37 (5H, s, Ph), 5.72 (1H, d, J=10.0 Hz, H-9), 5.21 (1H, dd, J=10.0, 1.9 Hz, H-8), 4.96 (1H, part A syst. AB, J=11.6 Hz, OCH₂Ph), 4.82 (1H, part B syst. AB, J=11.6 Hz, OCH₂Ph), 4.49 (1H, d, J=1.5 Hz, H-6), 4.10 (1H, t, J=1.9, 1.5 Hz, H-5), 3.32 (1H, part A syst. AB, J=13.8 Hz, H-3), 2.26 (1H, part A syst. AB, J=13.8 Hz, H-3), 0.80 (9H, s, C(CH₃)₃), 0.28 (9H, s, Si(CH₃)₃), 0.20 (9H, s, Si(CH₃)₃), 0.09 (3H, s, SiCH₃), 0.04 (3H, s, SiCH₃); ¹³C-NMR (75 MHz, CDCl₃): δ 165.7, 135.6, 132.6, 129.3, 128.8, 128.7, 126.9, 119.7, 78.8, 78.6, 69.8, 67.0, 65.6, 40.6, 25.8, 18.2, 1.4, 0.2, −4.1, −4.6, −7.0; IR (NaCl, CCl₄): ν 2957, 2891, 2855, 1785, 1255, 1101, 878, 843, 753 cm⁻¹; LRMS (API-ES⁺): m/z 1171 (2M+Na)⁺, 598 (M+Na)⁺, 575 (M+H)⁺.

### Example 7

### Preparation of rac-(4R,5S,6S,7S)-1-benzyloxy-7-cyano-5,6,7-tris(trimethylsilyloxy)-1-azaspiro[3.5]nona-8-ene-2-one (8)

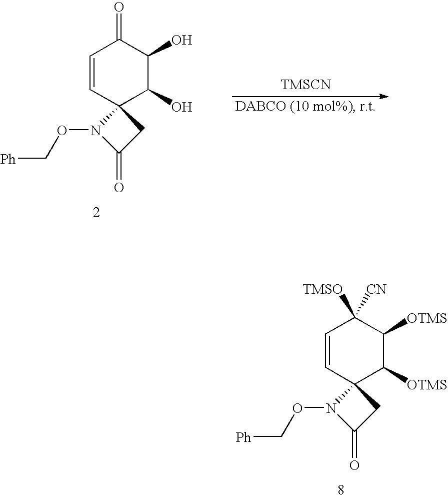

To a mixture of rac-(4R,5S,6S)-1-benzyloxy-5,6-dihydroxy-1-azaspiro[3.5]nona-8-ene-2,7-dione (2) (44 mg, 0.152 mmol) and trimethylsilyl cyanide (186 μl, 1.368 mmol) at room temperature (cooled with water bath) was slowly added DABCO (2 mg, 0.015 mmol). The mixture was stirred at room temperature for 14 h and then concentrated under reduced pressure. The residue was purified by silica gel column chromatography (hexane-AcOEt, 5:1) to give rac-(4R,5S,6S,7S)-1-benzyloxy-7-cyano-5,6,7-tris(trimethylsilyloxy)-1-azaspiro[3.5]nona-8-ene-2-one (8) as a yellow solid (51 mg, 62%).

R_{f}=0.50 (TLC, hexane-AcOEt, 3:1); yield, 62%; yellow solid; ¹H-NMR (200 MHz, CDCl₃): δ 7.37 (5H, s br., Ph), 5.33-5.20 (2H, m, H-8 and H-9), 4.96 (1H, part A syst. AB, J=11.6 Hz, OCH₂ Ph), 4.81 (1H, part B syst. AB, J=11.6 Hz, OCH₂ Ph), 4.41 (1H, d, J=1.5 Hz, H-5 or H-6), 4.08 (1H, t, J=1.5 Hz, H-6 or H-5), 3.27 (1H, part A syst. AB, J=14.1 Hz, CH₂), 2.27 (1H, part B syst. AB, J=14.1 Hz, CH₂), 0.27 (9H, s, Si(CH₃)₃), 0.20 (9H, s, Si(CH₃)₃), 0.08 (9H, s, Si(CH₃)₃); ¹³C-NMR (75 MHz, CDCl₃): δ 165.9, 135.6, 132.8, 129.2, 128.8, 128.7, 126.7, 119.6, 78.8, 78.4, 69.4, 66.8, 65.5, 40.3, 1.3, 0.4, 0.2; IR (NaCl, CCl₄): 3391, 2958, 2898, 2222, 1785, 1455, 1400, 1253, 1168, 1104, 1030, 880, 843, 752 cm⁻¹; LRMS (API-ES⁺): m/z 1087 (2M+Na)⁺, 555 (M+Na)⁺, 533 (M+H)⁺.

### Example 8

### Preparation of rac-(4R,5S,6S,7S)-1-benzyloxy-7-cyano-6-tert-butyldimethylsilyloxy-5,7-bis(methoxycarbonyloxy)-1-azaspiro[3.5]nona-8-ene-2-one (9)

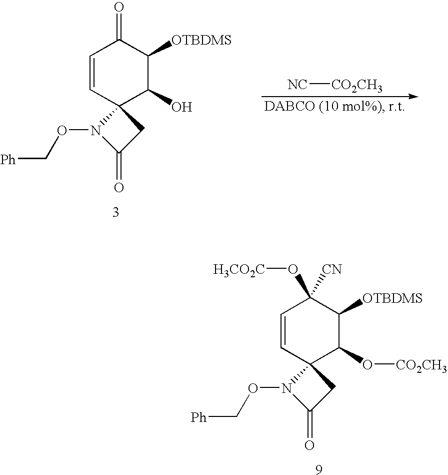

To a mixture of rac-(4R,5S,6S)-1-benzyloxy-6-tert-butyldimethylsilyloxy-5-hydroxy-1-azaspiro[3.5]nona-8-ene-2,7-dione (3) (95 mg, 0.235 mmol) and methylcyanoformate (169 μl, 2.115 mmol) was added at room temperature DABCO (3 mg, 0.023 mmol). The mixture was stirred at room temperature for 14 h and then concentrated under reduced pressure. The residue was triturated with Et₂O, filtered and the solvent was evaporated under reduced pressure to give rac-(4R,5S,6S,7S)-1-benzyloxy-7-cyano-6-tert-butyldimethylsilyloxy-5,7-bis(methoxycarbonyloxy)-1-azaspiro[3.5]nona-8-ene-2-one (9) as a brown oil (110 mg, 86%), was used in the next reaction without further purification.

R_{f}=0.65 (TLC, hexane-AcOEt, 1:1); yield, 86%; brown oil; ¹H-NMR (200 MHz, CDCl₃): δ 7.39 (5H, s br., Ph), 5.73 (1H, dd, J=10.3, 2.0 Hz, H-8 or H-9), 5.46 (1H, d, J=10.3 Hz, H-9 or H-8), 5.40 (1H, d, J=1.8 Hz, H-5 or H-6), 4.95 (1H, part A syst. AB, J=11.5 Hz, OCH₂ Ph), 4.87 (1H, part B syst. AB, J=11.5 Hz, OCH₂ Ph), 4.77 (1H, dd, J=2.0, 1.8 Hz, H-6 or H-5), 3.85 (3H, s, OCH₃), 3.84 (3H, s, OCH₃), 3.29 (1H, part A syst AB, J=14.4 Hz, CH₂), 2.44 (1H, part B syst. AB, J=14.4 Hz, CH₂), 0.79 (9H, s, C(CH₃)₃), 0.07 (3H, s, SiCH₃), 0.04 (3H, s, SiCH₃); ¹³C-NMR (75 MHz, CDCl₃): δ 165.5, 154.2, 152.9, 135.2, 133.9, 129.4, 129.3, 129.0, 128.9, 123.2, 115.1, 79.1, 72.5, 72.3, 70.9, 63.1, 55.7, 55.5, 41.7, 25.5, 25.1, 17.9, −4.5, −5.5; IR (NaCl, CCl₄): 3355, 2958, 2927, 2855, 2233, 1786, 1763, 1442, 1274, 1255, 1155, 1050, 834, 783 cm⁻¹; LRMS (API-ES⁺): m/z 1115 (2M+Na)⁺, 569 (M+Na)⁺, (M+H)⁺; LRMS (EI): m/z 546 (M⁺, 2), 489(5), 455(1), 413(3), 337(18), 323(4), 295(17), 216(10), 190(17), 133(16), 91 (100).

### Example 9

### Preparation of rac-(4R,5S,6S,7R,8S,9S)-1-benzyloxy-6-ter-butyldimethylsilyloxy-7-cyano-8,9-dihydroxy-5,7-bis(trimethylsilyloxy)-1-azaspiro[3.5]nona-2-one (10a) and rac-(4R,8S,9S,7S,5S)-1-benzyloxy-8-tert-butyldimethylsilyloxy-7-cyano-5-dihydroxy-7,9-bis(trimethylsilyloxy)-1-azaspiro[3.5]nona-2,6-dione (10b)

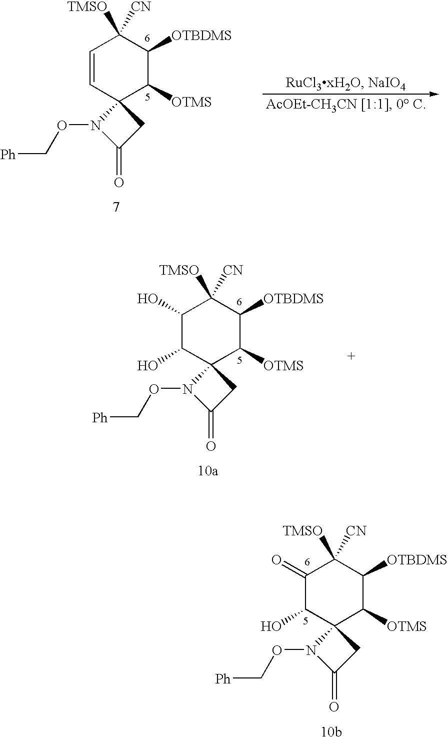

To a solution of rac-(4R,5S,6S,7S)-1-benzyloxy-6-tert-butyldimethylsilyloxy-7-cyano-5,7-bis(trimethylsilyloxy)-1-azaspiro[3.5]nona-8-ene-2-one (7) (100 mg, 0.174 mmol) in AcOEt (1 ml) and CH₃CN (1 ml) was added at 0° C. with vigorous stirring a solution of ruthenium (III) chloride hydrate (9 mg, 0.043 mmol) and sodium periodate (71 mg, 0.331 mmol) in H₂O (0.35 ml). After 5 min the reaction the mixture was quenched with saturated aqueous sodium bisulfite solution (1.5 ml) and extracted with AcOEt (3×2 ml). The combined organic extracts were dried over Na₂SO₄, filtered and concentrated under reduced pressure. The residue was purified by silica gel column chromatography (hexane-AcOEt, 5:1) to give rac-(4R,5S,6S,7R,8S,9S)-1-benzyloxy-6-tert-butyldimethylsilyloxy-7-cyano-8,9-dihydroxy-5,7-bis(trimethylsilyloxy)-1-azaspiro[3.5]nona-2-one (10a) as a white solid (63 mg, 59%) and rac-(4R,8S,9S,7S,5S)-1-benzyloxy-8-tert-butyldimethylsilyloxy-7-cyano-5-dihydroxy-7,9-bis(trimethylsilyloxy)-1-azaspiro[3.5]nona-2,6-dione (10b) as a white solid (11 mg, 10%).

### rac-(4R,5S,6S,7R,8S,9S)-1-benzyloxy-6-tert-butyldimethylsilyloxy-7-cyano-8,9-dihydroxy-5,7-bis(trimethylsilyloxy)-1-azaspiro[3.5]nona-2-one (10a)

Rf=0.17 (TLC, hexane-AcOEt, 3:1); yield, 59%; white solid; ¹H-NMR (200 MHz, CDCl₃): δ 7.47-7.33 (5H, m, Ph), 5.21 (1H, part A syst. AB, J=10.1 Hz, OCH₂Ph), 5.02 (1H, part B syst. AB, J=10.1 Hz, OCH₂Ph), 4.74 (1H, d, J=2.1 Hz, H-8 or H-7), 4.10 (1H, d, J=2.1 Hz, H-7 or H-8), 3.92 (1H, m, H-6 or H-5), 3.83 (1H, m, H-5 or H-6), 3.27 (1H, part A syst. AB, J=14.0 Hz, H-3), 3.27 (1H, s, OH), 2.15 (1H, part B syst. AB, J=14.0 Hz, H-3), 2.01 (1H, s, OH), 0.86 (9H, s, C(CH₃)₃), 0.30 (9H, s, Si(CH₃)₃), 0.14 (3H, s, SiCH₃), 0.13 (9H, s, Si(CH₃)₃), 0.11 (3H, s, SiCH₃); ¹³C-NMR (50 MHz, CDCl₃): δ 166.0, 134.9, 128.9, 128.5, 119.3, 97.1, 78.9, 78.7, 74.8, 73.9, 70.9, 68.8, 65.2, 38.6, 26.1, 18.3, 1.8, 0.4, −3.8, −5.0; IR (KBr): ν 3434, 3028, 2957, 2927, 2898, 2855, 2152, 1762, 1630, 1253, 1169, 1107, 846, 740 cm⁻¹; LRMS (API-ES⁺): m/z 1240 (2M+Na)⁺, 631 (M+Na)⁺, 609 (M+H)⁺.

The structure of compound 10a was confirmed via X-ray difraction, using a ENRAF-NONIUS CAD-4 difractometer. The crystal data and atomic coordinates obtained are as follows:

Crystal data

| Empirical formula | C₂₈H₄₈N₂O₇Si₃ |
|---|---|
| Formula weight | 608.95 |
| Temperature | 293(2) K |
| Wavelength (Å) | 0.71073 |
| Crystal system, space group | Monoclinic, P21/n |
| Unit cell dimensions | a = 13.862(13) Å |
| | b = 19.504(7) |
| | c = 26.674(12) |
| | β = 102.28(5)° |
| Volume | 7047(8) Å³ |
| Z, Calculated density | 8, 1.148 Mg/m³ |
| μ (mm⁻¹) | 0.176 |
| F(000) | 2624 |

Atomic coordinates (×10⁴) and equivalent isotropic displacement
parameters (Å² × 10³). U(eq) is defined as one third of the trace
of the orthogonalized Uᵢⱼ tensor.

| | x | y | z | U(eq) |
|---|---|---|---|---|
| Si(1A) | 10695(1) | 7051(1) | 2392(1) | 55(1) |
| Si(2A) | 7587(1) | 8399(1) | 2027(1) | 52(1) |
| Si(3A) | 4928(1) | 6645(1) | 2085(1) | 60(1) |
| N(1A) | 5967(2) | 5965(2) | 1053(1) | 53(1) |
| N(2A) | 8846(3) | 5566(2) | 2735(1) | 75(1) |
| O(1A) | 5483(2) | 5429(1) | 1228(1) | 53(1) |
| O(2A) | 4846(2) | 6471(1) | 383(1) | 68(1) |
| O(3A) | 9469(2) | 7063(1) | 2308(1) | 42(1) |
| O(4A) | 7694(2) | 7604(1) | 1834(1) | 43(1) |
| O(5A) | 5926(2) | 6894(1) | 1887(1) | 53(1) |
| O(6A) | 7818(2) | 5321(1) | 1505(1) | 64(1) |
| O(7A) | 9576(2) | 5990(1) | 1585(1) | 60(1) |
| C(1A) | 6855(3) | 6351(2) | 1323(1) | 43(1) |
| C(2A) | 7820(3) | 5984(2) | 1294(1) | 48(1) |
| C(3A) | 8721(2) | 6395(2) | 1566(1) | 42(1) |
| C(4A) | 8702(2) | 6612(2) | 2106(1) | 39(1) |
| C(5A) | 7700(2) | 6992(2) | 2109(1) | 42(1) |
| C(6A) | 6820(2) | 6536(2) | 1868(1) | 42(1) |
| C(7A) | 6499(3) | 6889(2) | 905(1) | 53(1) |
| C(8A) | 5609(3) | 6442(2) | 711(2) | 50(1) |
| C(9A) | 5595(3) | 4786(2) | 977(2) | 63(1) |
| C(10A) | 4594(3) | 4482(2) | 756(2) | 51(1) |
| C(11A) | 4336(3) | 3874(2) | 904(2) | 68(1) |
| C(12A) | 3441(4) | 3584(3) | 688(2) | 89(2) |
| C(13A) | 2780(4) | 3938(3) | 327(2) | 86(2) |
| C(14A) | 3017(3) | 4564(3) | 172(2) | 79(1) |
| C(15A) | 3953(3) | 4837(2) | 391(2) | 71(1) |
| C(16A) | 5153(3) | 5843(2) | 2442(2) | 98(2) |
| C(17A) | 4619(3) | 7337(2) | 2493(2) | 92(2) |
| C(18A) | 3888(3) | 6548(2) | 1521(2) | 82(1) |
| C(19A) | 8776(3) | 6012(2) | 2455(2) | 45(1) |
| C(20A) | 11046(3) | 7809(2) | 2800(2) | 81(1) |
| C(21A) | 11117(3) | 7183(3) | 1783(2) | 102(2) |
| C(22A) | 11241(3) | 6269(2) | 2712(2) | 91(2) |
| C(23A) | 6270(3) | 8593(3) | 2020(2) | 119(2) |
| C(24A) | 8290(3) | 8530(2) | 2683(2) | 92(2) |
| C(25A) | 8055(4) | 8927(2) | 1574(2) | 77(1) |
| C(26A) | 9115(6) | 8777(4) | 1583(3) | 205(4) |
| C(27A) | 7972(4) | 9686(3) | 1668(2) | 112(2) |
| C(28A) | 7407(6) | 8800(3) | 1022(2) | 184(3) |
| Si(1B) | 4427(1) | 8447(1) | −648(1) | 57(1) |
| Si(2B) | 1911(1) | 9027(1) | 370(1) | 50(1) |
| Si(3B) | −938(1) | 8029(1) | −710(1) | 60(1) |
| N(1B) | 548(2) | 6331(2) | −140(1) | 49(1) |
| N(2B) | 2032(3) | 7631(2) | −1577(2) | 75(1) |
| O(1B) | −195(2) | 6174(1) | −560(1) | 50(1) |
| O(2B) | −174(2) | 6229(1) | 572(1) | 64(1) |
| O(3B) | 3311(2) | 8340(1) | −525(1) | 48(1) |
| O(4B) | 2014(2) | 8260(1) | 122(1) | 38(1) |
| O(5B) | 53(2) | 7744(1) | −303(1) | 45(1) |
| O(6B) | 1976(2) | 6290(1) | −778(1) | 68(1) |
| O(7B) | 3774(2) | 6888(1) | −527(1) | 58(1) |
| C(1B) | 1315(2) | 6871(2) | −134(1) | 41(1) |
| C(2B) | 2243(3) | 6607(2) | −288(2) | 52(1) |
| C(3B) | 3022(2) | 7164(2) | −296(1) | 44(1) |
| C(4B) | 2617(2) | 7807(2) | −591(1) | 38(1) |
| C(5B) | 1699(2) | 8056(2) | −392(1) | 38(1) |
| C(6B) | 918(2) | 7495(2) | −447(1) | 38(1) |
| C(7B) | 1324(2) | 6916(2) | 445(1) | 46(1) |
| C(8B) | 437(3) | 6442(2) | 342(2) | 49(1) |
| C(9B) | −88(3) | 5480(2) | −750(2) | 76(1) |
| C(10B) | −764(4) | 5437(2) | −1254(2) | 66(1) |
| C(11B) | −1693(4) | 5175(3) | −1307(2) | 93(2) |
| C(12B) | −2359(5) | 5189(4) | −1798(3) | 128(2) |
| C(13B) | −2045(7) | 5450(4) | −2183(3) | 139(3) |
| C(14B) | −1127(7) | 5684(4) | −2147(3) | 139(3) |
| C(15B) | −495(4) | 5688(3) | −1679(3) | 102(2) |
| C(16B) | −643(3) | 8835(2) | −1012(2) | 107(2) |
| C(17B) | −1837(3) | 8172(3) | −296(2) | 106(2) |
| C(18B) | −1398(3) | 7408(2) | −1221(2) | 81(1) |
| C(19B) | 2296(3) | 7686(2) | −1144(2) | 51(1) |
| C(20B) | 4568(3) | 8043(2) | −1246(2) | 85(2) |
| C(21B) | 4520(3) | 9383(2) | −704(2) | 110(2) |
| C(22B) | 5402(3) | 8129(2) | −105(2) | 88(2) |
| C(23B) | 692(3) | 9112(2) | 542(2) | 91(2) |
| C(24B) | 2047(3) | 9695(2) | −94(2) | 81(1) |
| C(25B) | 2913(3) | 9061(2) | 946(2) | 65(1) |
| C(26B) | 2743(4) | 8529(3) | 1345(2) | 121(2) |
| C(27B) | 3931(3) | 8931(3) | 802(2) | 112(2) |
| C(28B) | 2956(4) | 9762(3) | 1216(2) | 101(2) |

### rac-(4R,8S,9S,7S,5S)-1-benzyloxy-8-tert-butyldimethylsilyloxy-7-cyano-5-dihydroxy-7,9-bis(trimethylsilyloxy)-1-azaspiro[3.5]nona-2,6-dione (10b)

Rf=0.33 (TLC, hexane-AcOEt, 3:1); yield, 10%; white solid; ¹H-NMR (200 MHz, CDCl₃): δ 7.43-7.31 (5H, m, Ph), 5.20 (1H, part A syst. AB, J=11.0 Hz, OCH₂Ph), 5.17 (1H, part B syst. AB, J=11.0 Hz, OCH₂Ph), 4.74 (1H, br. s, H-5), 4.62 (1H, d, J=2.3 Hz, H-8), 4.27 (1H, d, J=2.3 Hz, H-9), 3.83 (1H, br. s, OH), 3.46 (1H, part A syst. AB, J=13.2 Hz, H-3), 2.48 (1H, part B syst. AB, J=13.2 Hz, H-3), 0.91 (9H, S, C(CH₃)₃), 0.33 (9H, s, Si(CH₃)₃), 0.20 (3H, s, SiCH₃), 0.18 (3H, s, SiCH₃), 0.12 (9H, s, Si(CH₃)₃); ¹³C-NMR (75 MHz, CDCl₃): δ 202.4, 162.6, 134.7, 129.2, 128.6, 128.4, 117.0, 79.5, 78.4, 76.5, 75.8, 72.9, 66.5, 39.2, 25.9, 18.4, 1.4, 0.2, −4.2, −4.8; IR (KBr): ν 3449, 2957, 2898, 2855, 2111, 1768, 1755, 1734, 1631, 1256, 1182, 1144, 1080, 922, 847, 780 cm⁻¹; LRMS (API-ES⁺): m/z 1235 (2M+Na)⁺, 629 (M+Na)⁺, 606 (M+H)⁺.

### Example 10

### Preparation of rac-(4R,5S,6S,7S,8S,9S)-1-benzyloxy-6,8-bis(tert-butyldimethylsilyloxy)-7-cyano-9-hydroxy-5,7-bis(trimethylsilyloxy)-1-azaspiro[3.5]nona-2,7-dione (11)

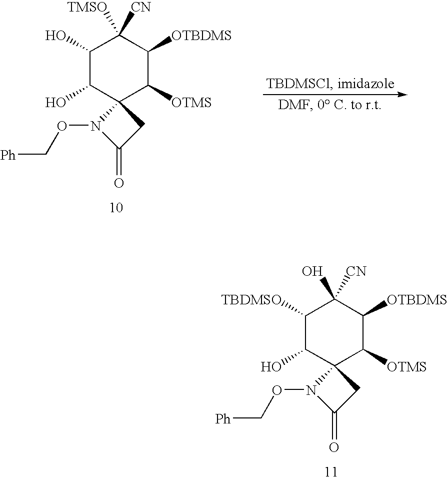

To a solution of rac-(4R,5S,6S,7S,8S,9S)-1-benzyloxy-6-tert-butyldimethylsilyloxy-7-cyano-8,9-dihydroxy-5,7-bis(trimethylsilyloxy)-1-azaspiro[3.5]nona-2,7-dione (10a) (63 mg, 0.103 mmol) and imidazole (8 mg, 0.124 mmol) in DMF (0.25 ml) was added at 0° C. a solution of tert-butyldimethylsilyl chloride (19 mg, 0.124 mmol) in DMF (0.5 ml). After 16 h at room temperature, the reaction was quenched with H₂O (2 ml) and the mixture extracted with AcOEt (3×4 ml). The combined extracts were washed with saturated aqueous CuSO₄ solution (2×8 ml) and brine (2×8 ml), dried over Na₂SO₄, filtered and concentrated under reduced pressure. The residue was purified by silica gel column chromatography (hexane-AcOEt, 5:1) to give rac-(4R,5S,6S,7S,8S,9S)-1-benzyloxy-6,8-bis(tert-butyldimethylsilyloxy)-7-cyano-9-hydroxy-5,7-bis(trimethylsilyloxy)-1-azaspiro[3.5]nona-2,7-dione (11) as a white solid (37 mg, 46%).

R_{f}=0.60 (TLC, hexane-AcOEt, 3:1); yield, 46%; white solid; ¹H-NMR (200 MHz, CO(CD₃)₂): δ 7.54-7.42 (2H, m, Ph), 7.41-7.29 (3H, m, Ph), 5.26 (1H, part A syst. AB, J=10.2 Hz, OCH₂Ph), 5.06 (1H, part B syst. AB, J=10.2 Hz, OCH₂Ph), 4.82 (1H, d, J=2.4 Hz, H-5), 4.70 (1H, d, J=2.4 Hz, OH), 4.27 (1H, d, J=2.4 Hz, H-6), 4.16 (1H, m, H-9), 3.99 (1H, s br., H-8), 3.23 (1H, part A syst. AB, J=13.7 Hz, CH₂), 2.89 (1H, part B syst. AB, J=13.7 Hz, CH₂), 0.91 (18H, s, C(CH₃)₃), 0.19 (6H, s, SiCH₃), 0.18 (6H, s, SiCH₃), 0.11 (18H, s, Si(CH₃)₃).

The structure of compound II was confirmed via X-ray difraction, using a MAR-3200 difractometer with an Image plate detector. The crystal data and atomic coordinates obtained are as follows:

Crystal data

| Empirical formula | C₂₈H₄₈N₂O₇Si₃ |
|---|---|
| Formula weight | 608.95 |
| Temperature | 293(2) K |
| Wavelength | 0.71073 Å |
| Crystal system, space group | Monoclinic, P21/c |
| Unit cell dimensions | a = 11.4160(10) Å |
| | b = 22.2850(10) |
| | c = 14.8440(10) |
| | β = 104.760(10)° |
| Volume | 3651.8(4) Å³ |
| Z, Calculated density | 4, 1.108 Mg/m³ |
| Absorption coefficient | 0.170 mm⁻¹ |
| F(000) | 1312 |

Atomic coordinates (×10⁴) and equivalent isotropic displacement
parameters (Å² × 10³). U(eq) is defined as one third of the trace
of the orthogonalized Uᵢⱼ tensor.

| | x | y | z | U(eq) |
|---|---|---|---|---|
| Si(1) | 1134(1) | 3283(1) | 4989(1) | 70(1) |
| Si(2) | 2580(2) | 1056(1) | 4724(1) | 82(1) |
| O(1) | 5491(3) | 1142(1) | 6946(2) | 63(1) |
| O(2) | 3641(3) | 1163(1) | 8128(2) | 72(1) |
| O(3) | 6292(3) | 2369(1) | 6568(2) | 66(1) |
| O(4) | 5967(3) | 3552(1) | 6484(2) | 65(1) |
| O(5) | 3912(3) | 3710(1) | 4978(2) | 61(1) |
| O(6) | 2353(2) | 2897(1) | 5505(2) | 52(1) |
| O(7) | 2765(3) | 1651(1) | 5389(2) | 63(1) |
| N(1) | 4663(3) | 1553(1) | 7066(2) | 57(1) |
| N(2) | 5761(4) | 2753(2) | 4304(3) | 78(1) |
| C(1) | 4282(3) | 2134(1) | 6595(2) | 47(1) |
| C(2) | 3716(4) | 2070(2) | 5549(2) | 56(1) |
| C(3) | 3279(4) | 2677(2) | 5134(2) | 53(1) |
| C(4) | 4319(3) | 3133(1) | 5292(2) | 45(1) |
| C(5) | 4899(4) | 3200(1) | 6351(2) | 52(1) |
| C(6) | 5266(4) | 2604(2) | 6831(2) | 54(1) |
| C(7) | 3362(4) | 2149(2) | 7204(2) | 53(1) |
| C(8) | 3865(4) | 1539(2) | 7575(2) | 55(1) |
| C(9) | 6566(4) | 1165(2) | 7734(3) | 60(1) |
| C(10) | 7607(4) | 888(2) | 7431(3) | 63(1) |
| C(11) | 8273(5) | 452(2) | 7949(3) | 86(2) |
| C(12) | 9307(6) | 238(2) | 7739(5) | 105(2) |
| C(13) | 9628(6) | 447(3) | 7014(5) | 112(2) |
| C(14) | 8913(6) | 856(3) | 6419(4) | 110(2) |
| C(15) | 7916(5) | 1088(2) | 6662(4) | 86(1) |
| Si(3) | 6281(7) | 4179(3) | 7068(4) | 92(2) |
| C(16) | 5060(30) | 4598(12) | 7100(20) | 197(16) |
| C(17) | 7050(20) | 4647(8) | 6249(18) | 192(10) |
| C(18) | 7380(30) | 4116(14) | 8044(15) | 280(20) |
| Si(3′) | 6170(6) | 4279(2) | 6633(3) | 70(1) |
| C(16′) | 4821(16) | 4634(7) | 6916(14) | 71(4) |
| C(17′) | 6414(14) | 4600(5) | 5535(9) | 108(4) |
| C(18′) | 7546(17) | 4393(9) | 7372(17) | 166(8) |
| C(19) | 5182(4) | 2932(2) | 4756(3) | 57(1) |
| C(20) | 1298(6) | 4079(3) | 5313(5) | 129(2) |
| C(21) | 827(6) | 3213(2) | 3705(3) | 102(2) |
| C(22) | −113(7) | 2974(4) | 5416(5) | 115(2) |
| C(23) | −345(14) | 2376(6) | 5497(11) | 116(4) |
| C(24) | 278(18) | 3182(6) | 6541(12) | 99(5) |
| C(25) | −1220(40) | 3232(13) | 5090(30) | 200(16) |
| C(23′) | −354(14) | 2311(6) | 4869(10) | 123(5) |
| C(24′) | 81(19) | 2822(9) | 6373(14) | 129(6) |
| C(25′) | −1280(20) | 3451(10) | 5030(19) | 98(5) |
| C(26) | 3829(15) | 495(7) | 5045(10) | 136(5) |
| C(27) | 1343(16) | 659(8) | 4967(12) | 151(6) |
| C(28) | 2645(18) | 1260(6) | 3583(8) | 129(6) |
| C(26′) | 3840(30) | 941(12) | 4400(20) | 284(16) |
| C(27′) | 2680(20) | 429(6) | 5448(11) | 153(6) |
| C(28′) | 1310(30) | 1185(11) | 3640(20) | 370(30) |

### Example 11

### General procedure for the preparation of the silylcyanohydrin 12a and 12b from the spiro β-lactams 1a and 1b, respectively

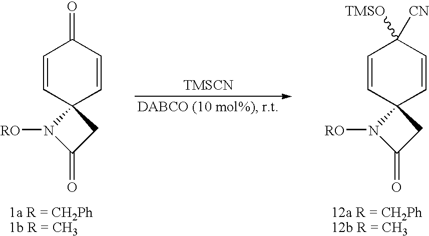

To a mixture of Spiro β-lactams 1a-b (0.588 mmol) and trimethylsilyl cyanide (1.763 mmol) at room temperature (cooled with water bath) was slowly added DABCO (10 mol %). The mixture was stirred at room temperature until the disappearance of starting material by TLC (hexane-AcOEt, 1:1) was observed (the time required was 6 h for 1a and 24 h for 1b), and then concentrated under reduced pressure. The silylcyanohydrin 12a-b isolated by procedure could be used without further purification (the purification by silica gel column chromatography to give mixtures of silylcyanohydrin 12a-b and starting material 1a-b).

rac-(4R,7S) and (4R,7R)-1-benzyloxy-7-cyano-1-azaspiro[3.5]nona-5,8-diene-2-one (12a) R_{f}=0.64 (TLC, hexane-AcOEt, 1:1); yield, 88%; brown solid; ¹H-NMR (200 MHz, CDCl₃): δ 7.37 (5H, m, Ph), 7.35 (5H, m, Ph), 6.09 (2H, part A A′ syst. AA′BB′, J_{5,6}=10.1 Hz, CH═CHCO), 6.06 (2H, part AA′ syst. AA ‘BB’, J_{5,6}=10.1 Hz, CH═CHCO), 5.87 (2H, part BB′ syst. AA′ BB′, J_{6,5}=10.1 Hz, CH═CHCO), 5.79 (2H, part BB′ syst. AA′ BB′, J_{6,5}=10.1 Hz, CH═CHCO), 4.89 (2H, s, OCH₂Ph), 4.87 (2H, s, OCH₂Ph), 2.75 (2H, S, CH₂), 2.73 (2H, s, CH₂), 0.26 (9H, s, Si(CH₃)₃), 0.19 (9H, s, Si(CH₃)₃).

rac-(4R,7S) and 4R,7R)-1-methoxy-7-cyano-1-azaspiro[3.5]nona-5,8-diene-2-one (12b) R_{f}=0.61 (TLC, hexane-AcOEt, 1:2); yield, 64%; brown solid; ¹H-NMR (200 MHz, CDCl₃): δ 6.26 (2H, part AA′ syst. AA′ BB′, J_{5,6}=10.1 Hz, CH═CHCO), 6.21 (2H, part AA′ syst. AA′BB′, J_{5,6}=10.1 Hz, CH═CHCO), 6.09 (2H, part BB′ syst. AA′BB′, J_{6,5}=10.1 Hz, CH═CHCO), 6.08 (2H, part BB′ syst. AA′BB′, J_{6,5}=10.1 Hz, CH—CHCO), 3.76 (6H, s, OCH₃), 2.81 (2H, s, CH₂), 2.80 (2H, s, CH₂), 0.24 (9H, s, Si(CH₃)₃), 0.23 (9H, s, Si(CH₃)₃).

### Example 12

### Preparation of rac-(4R,5S,6S)-1-benzyloxy-5,6-dimethylmethylendioxy-1-azaspiro[3.5]nona-8-ene-2,7-dione (13)

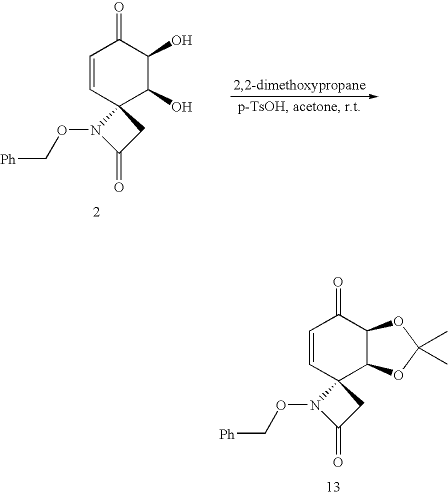

To a stirred solution of rac-(4R,5S,6S)-1-benzyloxy-5,6-dihydroxy-1-azaspiro[3.5]nona-8-ene-2,7-dione (2) (109 mg, 0.377 mmol) and 2,2-dimethoxypropane (0.24 ml, 1.885 mmol) in dry acetone (0.75 ml) was added at room temperature catalytic amount of p-TsOH (1% mmol). The resulting mixture was stirred at room temperature for 18 h, then quenched with saturated aqueous Na₂CO₃ solution (1 ml) and extracted with AcOEt (3×2 ml). The combined organic extracts were washed with brine (3 ml), dried over Na₂SO₄, filtered and concentrated under reduced pressure. The residue was purified by silica gel column chromatography (hexane-AcOEt, 5:2) to give rac-(4R,5S,6S)-1-benzyloxy-5,6-dimethylmethylendioxy-1-azaspiro[3.5]nona-8-ene-2,7-dione (13) as a solid (52 mg, 42%).

R_{f}=0.50 (TLC, hexane-AcOEt, 1:1); yield, 42%; solid; ¹H-NMR (200 MHz, CDCl₃): δ 7.50-7.22 (5H, m, Ph), 6.28 (1H, part A syst. AB, J_{9,8}=9.9 Hz, H-9), 5.91 (1H, part B syst. AB, J_{8,9}=9.9 Hz, H-8), 4.95 (1H, part A syst. AB, J=11.4 Hz, OCH₂Ph), 4.85 (1H, part B syst. AB, J=11.4 Hz, OCH₂Ph), 4.27 (1H, part A syst. AB, J=5.1 Hz, H-5 or H-6), 4.11 (1H, part B syst. AB, J=5.1 Hz, H-6 or H-5), 6.37 (1H, part A syst. AB, J=15.0 Hz, CH₂), 2.79 (1H, part B syst. AB, J=15.0 Hz, CH₂), 1.34 (3H, s, CH₃), 1.27 (3H, s, CH₃).

### Example 13

### Preparation of rac-(4R,5S,6S,7S)-1-benzyloxy-6-tert-butyldimethylsilyloxy-7,10-epoxy-7-methyl-5-trimethylsilyloxy-1-azaspiro[3.5]nona-8-en-2-one (14)

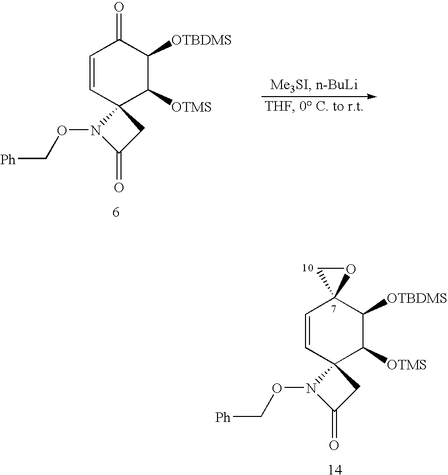

To a stirred suspension of powdered trimethylsulfonium iodide (52 mg, 0.252 mmol) in THF (0.9 ml) was added dropwise at 0° C. n-butyl lithium (0.16 ml, 1.6 M solution in hexane, 0.252 mmol). After stirring of the mixture for 15 min at this temperature, only a slight precipitate remained; a solution of rac-(4R,5S,6S)-1-benzyloxy-6-tert-butyldimethylsilyloxy-5-trimethylsilyloxy-1-azaspiro[3.5]nona-8-en-2,7-dione (6) (100 mg, 0.210 mmol) in THF (0.6 ml) was added dropwise. The mixture was stirred for 30 min at 0° C., and then for 2 h at room temperature. The reaction was quenched with Na₂HPO₄ 0.1 M buffer (2.5 ml) and AcOEt (2.5 ml). The layers were separated and aqueous phase was extracted with AcOEt (3×5 ml). The combined extracts were dried over Na₂SO₄, filtered and concentrated under reduced pressure. The residue was purified by silica gel column chromatography (hexane-AcOEt, 8:1) to give rac-(4R,5S,6S,7S)-1-benzyloxy-6-tert-butyldimethylsilyloxy-7,10-epoxy-7-methyl-5-trimethylsilyloxy-1-azaspiro[3.5]nona-8-en-2-one (14) as a low melting point white solid (84 mg, 82%).

R_{f}=0.50 (TLC, hexane-AcOEt, 3:1); yield, 82%; white solid; ¹H-NMR (300 MHz, CDCl₃): δ 7.34 (5H, m, Ph), 5.52 (1H, d, J=10.1 Hz, H-9), 5.06 (1H, dd, J=10.1, 1.9 Hz, H-8), 5.00 (1H, part A syst. AB, J=11.5 Hz, OCH₂Ph), 4.84 (1H, part B syst. AB, J=11.5 Hz, OCH₂Ph), 4.17 (1H, d, J=1.9 Hz, H-6), 3.50 (1H, t, J=1.9 Hz, H-5), 3.43 (1H, part A syst. AB, J=13.9 Hz, H-3), 2.87 (1H, part A syst. AB, J=5.0 Hz, H-10), 2.73 (1H, part B syst. AB, J=5.0 Hz, H-10), 2.34 (1H, part B syst. AB, J=13.9 Hz, H-3), 0.83 (9H, s, C(CH₃)₃), 0.14 (9H, s, Si(CH₃)₃), 0.04 (3H, s, SiCH₃), 0.02 (3H, s, SiCH₃); ¹³C-NMR (75 MHz, CDCl₃): δ 165.9, 135.7, 133.1, 129.6, 128.9, 128.6, 128.4, 78.7, 77.4, 68.7, 66.3, 58.9, 53.0, 40.7, 25.7, 18.2, 0.2, −4.9, −5.0; IR (NaCl, CCl₄): σ 3028, 2956, 2857, 1778, 1472, 1410, 1253, 1152, 1116, 987, 930, 879, 839, 778 cm⁻¹; LRMS (API-ES⁺): m/z 1001 (2M+Na)⁺, 512 (M+Na)⁺, 490 (M+H)⁺.

### Example 14

### Preparation of rac-(4R,5S,6S)-1-benzyloxy-6-tert-butyldimethylsilyloxy-7-methylen-5-trimethylsilyloxy-1-azaspiro[3.5]nona-8-en-2-one (15)

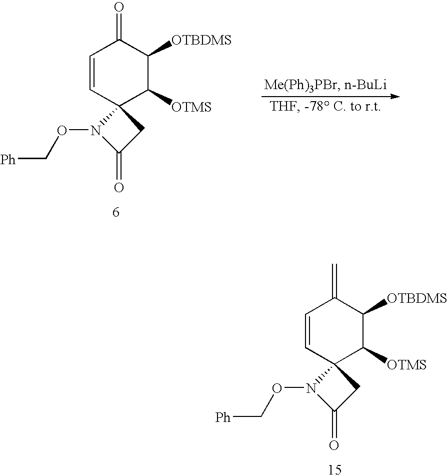

To a stirred suspension of methyltriphenylphosphonium (292 mg, 0.802 mmol) in THF (7.5 ml) was added dropwise at −78° C. n-butyl lithium (0.5 ml, 1.6 M solution in hexane, 0.802 mmol) and the resulting light yellow solution was stirred for 15 min at this temperature. Then, a solution of rac-(4R,5S,6S)-1-benzyloxy-6-tert-butyldimethylsilyloxy-5-trimethylsilyloxy-1-azaspiro[3.5]nona-8-en-2,7-dione (6) (381 mg, 0.802 mmol) in THF (7.5 ml) was added dropwise and the mixture was stirred for 1 h at room temperature. The reaction was quenched at 0° C. with H₂O (5 ml), the layers were separated and aqueous phase was extracted with AcOEt (3×10 ml). The combined extracts were dried over MgSO₄, filtered and concentrated under reduced pressure. The residue was purified by silica gel column chromatography (hexane-AcOEt, 8:1) to give rac-(4R,5S,6S)-1-benzyloxy-6-tert-butyldimethylsilyloxy-7-methylen-5-trimethylsilyloxy-1-azaspiro[3.5]nona-8-en-2-one (15) as a colourless oil (257 mg, 68%).

R_{f}=0.58 (TLC, hexane-AcOEt, 3:1); yield, 68%; colourless oil; ¹H-NMR (300 MHz, CDCl₃): δ7.34 (5H, m, Ph), 5.97 (1H, part A syst. AB, J=13.8 Hz, H-9), 5.38 (1H, part B syst. AB, J=13.8 Hz, H-8), 5.01 (2H, br. s, CH₂═C), 4.98 (1H, part A syst. AB, J=11.7 Hz, OCH₂Ph), 4.87 (1H, part B syst. AB, J=11.7 Hz, OCH₂Ph), 4.28 (1H, br. s, H-6), 4.06 (1H, br. s, H-5), 3.47 (1H, part A syst. AB, J=14.1 Hz, H-3), 2.28 (1H, part B syst. AB, J=14.1, H-3), 0.80 (9H, s, C(CH₃)₃), 0.15 (9H, s, Si(CH₃)₃), 0.07 (3H, s, SiCH₃), −0.01 (3H, s, SiCH₃); ¹³C-NMR (75 MHz, CDCl₃): δ 166.0, 144.3, 135.6, 130.6, 129.3, 128.8, 128.6, 128.4, 115.6, 78.6, 76.1, 69.2, 66.6, 40.3, 25.6, 18.0, 0.3, −4.6, −4.7; IR (NaCl, CCl₄): ν 3028, 2956, 2927, 2855, 1779, 1656, 1611, 1472, 1367, 1252, 1138, 1101, 988, 883, 840 cm⁻¹; LRMS (API-ES⁺): m/z 969 (2M+Na)⁺, 496 (M+Na)⁺, 474 (M+H)⁺.

### Example 15

### Preparation of rac-(4R,5S,6S,7R)-1-benzyloxy-6-tert-butyldimethylsilyloxy-7-hydroxy 7-hydroxymethyl-5-trimethylsilyloxy-1-azaspiro[3.5]nona-8-en-2-one (16)

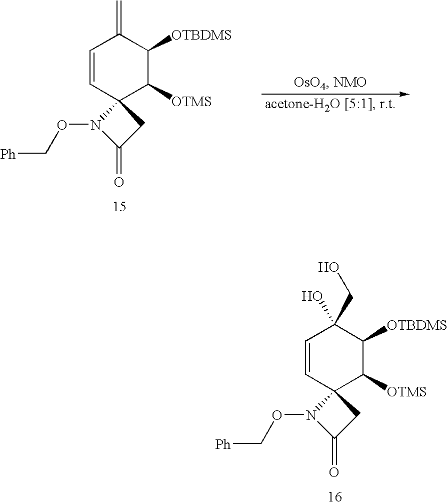

To a solution of rac-(4R,5S,6S)-1-benzyloxy-6-tert-butyldimethylsilyloxy-7-methylen-5-trimethylsilyloxy-1-azaspiro[3.5]nona-8-en-2-one (15) (235 mg, 0.496 mmol) in acetone (4 ml) was added sequentially at room temperature H₂O (0.8 ml), N-methylmorpholine N-oxide (132 mg, 1.091 mmol) and osmium tetroxide (0.37 ml, 2.5 wt. % solution in 2-methyl-2-propanol, 0.030 mmol). The resulting mixture was stirred at room temperature until the reaction was complete (16 h, TLC monitoring, hexane-AcOEt, 3:1), and then quenched with 10% aqueous Na₂S₂O₃ solution (1.5 ml). After 15 min, the mixture was extracted with AcOEt (3×4 ml). The combined organic extracts were dried over Na₂SO₄, filtered and concentrated under reduced pressure to give rac-(4R,5S,6S,7R)-1-benzyloxy-6-tert-butyldimethylsilyloxy-7-hydroxy-7-hydroxymethyl-5-trimethylsilyloxy-1-azaspiro[3.5]nona-8-en-2-one (16) as a low melting point pale yellow solid (240 mg, 95%).

Rf=0.13 (TLC, hexane-AcOEt, 3:1), 0.64 (TLC, hexane-AcOEt, 1:1); yield, 95%; pale yellow solid; ¹H-NMR (300 MHz, CDCl₃): δ 7.41-7.32 (5H, m, Ph), 5.47 (1H, d, J=9.9 Hz, H-9), 5.41 (1H, d, J=9.9 Hz, H-8), 5.01 (1H, part A syst. AB, J=11.5 Hz, OCH₂Ph), 4.84 (1H, part B syst. AB, J=11.5 Hz, OCH₂Ph), 4.48 (1H, br. s, H-6 or H-5), 3.83 (1H, br. s, H-5 or H-6), 3.67 (1H, br. d, J=11.1 Hz, CH₂—OH), 3.45 (1H, d, J=11.1 Hz, CH₂, —OH), 3.24 (1H, br. d, J=12.4 Hz, H-3), 2.26 (1H, br. d, J=12.4 Hz, H-3), 2.04 (2H, s br., OH), 0.81 (9H, s, C(CH₃)₃), 0.19 (9H, s, Si(CH₃)₃), 0.13 (3H, S, SiCH₃), 0.08 (3H, s, SiCH₃); ¹³C-NMR (75 MHz, CDCl₃): δ 165.3, 133.4, 129.5, 129.2, 128.8, 128.6, 128.5, 78.8, 76.2, 73.7, 68.0, 66.5, 65.8, 40.2, 26.0, 18.1, 0.4, −2.8, −5.4; IR (NaCl, CCl₄): ν 3434, 3028, 2956, 2858, 1755, 1478, 1462, 1456, 1411, 1389, 1362, 1253, 1141, 1090, 918, 882, 837, 756 cm⁻¹; LRMS (API-ES⁺): m/z 1037 (2M+Na)⁺, 530 (M+Na)⁺, 508 (M+H)⁺.

### Example 16

### Preparation of rac-(4R,5S,6S,9R)-1-benzyloxy-6-ted-butyldimethylsilyloxy-9-cyano-5-trimethylsilyloxy-1-azaspiro[3.5]nona-8-en-2,7-dione (17)

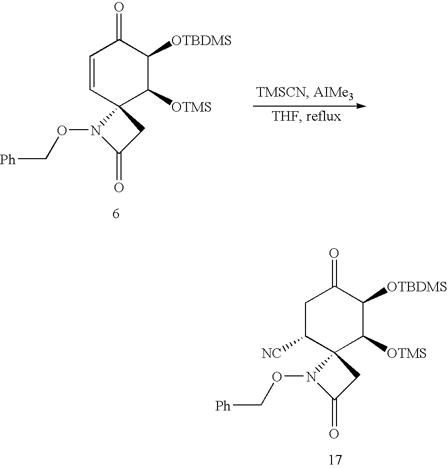

To a stirred solution of cyanotrimethyl silane (56 μl, 0.411 mmol) and trimethylaluminium (0.19 ml, 2.0 M solution in toluene, 0.374 mmol) in THF (0.5 ml) was added at room temperature a solution of rac-(4R,5S,6S)-1′-benzyloxy-6-tert-butyldimethylsilyloxy-5-trimethylsilyloxy-1-azaspiro[3.5]nona-8-en-2,7-dione (6) (89 mg, 0.187 mmol) in THF (0.5 ml). The resulting mixture was stirred at refiux for 24 h and then concentrated under reduced pressure. The residue was dissolved in toluene (2 ml), was washed with cold NH₄Cl sat. (1 ml) and Na₂HPO₄ 0.1 M buffer (1 ml), dried over Na₂SO₄, filtered and concentrated under reduced pressure. The residue was purified by silica gel column chromatography (hexane-AcOEt, 5:1) to give rac-(4R,5S,6S,9R)-1-benzyloxy-6-tert-butyldimethylsilyloxy-9-cyano-5-trimethylsilyloxy-1-azaspiro[3.5]nona-8-en-2,7-dione (17) as a low melting point white solid (21 mg, 22%).

R_{f}=0.41 (TLC, hexane-AcOEt, 3:1); yield, 22%; white solid; ¹H-NMR (300 MHz, CDCl₃): δ 7.44-7.30 (5H, m, Ph), 5.31 (1H, part A syst. AB, J=10.7 Hz, OCH₂Ph), 5.11 (1H, part B syst. AB, J=10.7 Hz, OCH₂Ph), 4.71 (1H, d, J=2.2 Hz, H-6), 4.13 (1H, d, J=2.2 Hz, H-5), 3.66 (1H, dd, J=13.4, 5.4 Hz, H-9), 2.92 (1H, part A syst. AB, J=14.5 Hz, H-3), 2.76 (1H, part B syst. AB, J=14.5 Hz, H-3), 2.63 (1H, dd, J=13.4, 5.4 Hz, H-8), 2.21 (1H, t, J=13.4 Hz, H-8), 0.85 (9H, s, C(CH₃)₃), 0.09 (9H, s, Si(CH₃)₃), 0.08 (3H, s, SiCH₃), −0.06 (3H, s, SiCH₃); ¹³C-NMR (75 MHz, CDCl₃): δ 201.0, 164.1, 133.9, 129.5, 129.4, 128.9, 117.1, 80.8, 78.9, 78.0, 67.3, 42.2, 38.3, 32.0, 25.8, 18.5, 0.5, −4.8, −5.6; IR (KBr): ν 3425, 2956, 2927, 2855, 2233, 1772, 1631, 1454, 1364, 1255, 1107, 1064, 841 cm⁻¹; LRMS (API-ES⁺): m/z 525 (M+Na)⁺, 503 (M+H)⁺.

### Example 17

### Preparation of rac-(4R,5S,6S,8S,9S)-1-benzyloxy-6-tert-butyldimethylsilyloxy-8,9-dihydroxy-5-trimethylsilyloxy-1-azaspiro[3.5]nonan-2,7-dione (18)

### Method A.

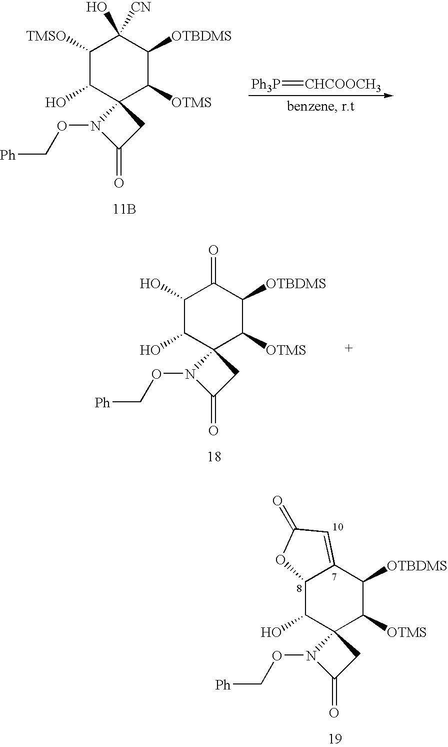

To a solution of rac-(4R,5S,6S,7S,8S,9S)-1-benzyloxy-6-tert-butyldimethylsilyloxy-7-cyano-7,9-dihydroxy-5,8-bis(trimethylsilyloxy)-1-azaspiro[3.5]nonan-2-one (11B) (58 mg, 0.095 mmol) in benzene (1.0 ml) was added at room temperature a solution of methyl (triphenylphosphoranylidene)acetate (76 mg, 0.228 mmol) in benzene (2 ml). The mixture was stirred to reflux for 11 h. The reaction was quenched with Na₂HPO₄ 0.1 M buffer (3 ml) and AcOEt (3 ml). The layers were separated and aqueous phase was extracted with AcOEt (3×6 ml). The combined extracts were dried over Na₂SO₄, filtered and concentrated under reduced pressure. The residue was purified by silica gel column chromatography (hexane-AcOEt, 4:1) to give rac-(4R,5S,6S,8S,9S)-1-benzyloxy-6-tert-butyldimethylsilyloxy-8,9-dihydroxy-5-trimethylsilyloxy-1-azaspiro[3.5]nonan-2,7-dione (18) as a white solid (19 mg, 38%) and rac-(4R,5S,6S,8S,9S)-1-benzyloxy-6-tert-butyldimethylsilyloxy-9-hydroxy-7-methylen-5-trimethylsilyloxy-1-azaspiro[3.5]nonan-2-on-10,8-carbolactone (19) as a white solid (10 mg, 20%).

### Method B.

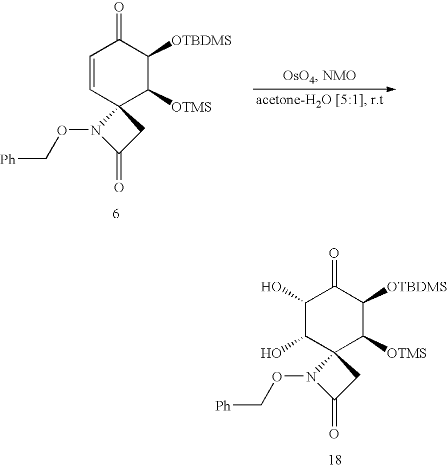

To a solution of rac-(4R,5S,6S)-1-benzyloxy-6-tert-butyldimethylsilyloxy-5-trimethylsilyloxy-1-azaspiro[3.5]nona-8-en-2,7-dione (6) (49 mg, 0.103 mmol) in acetone (0.5 ml) was added sequentially at room temperature H₂O (0.1 ml), N-methylmorpholine N-oxide (26 mg, 0.227 mmol) and osmium tetroxide (0.077 ml, 2.5 wt. % solution in 2-methyl-2-propanol, 0.006 mmol). The resulting mixture was stirred at room temperature until the reaction was complete (15 h, TLC monitoring, hexane-AcOEt, 1:1), and then quenched with 10% aqueous Na₂S₂O₃ solution (0.2 ml). After 20 min, the mixture was extracted with AcOEt (5×2 ml). The combined organic extracts were dried over Na₂SO₄, filtered and concentrated under reduced pressure. The residue was purified by silica gel column chromatography (hexane-AcOEt, 2:1) to give rac-(4R,5S,6S,8S,9S)-1-benzyloxy-6-tert-butyldimethylsilyloxy-8,9-dihydroxy-5-trimethylsilyloxy-1-azaspiro[3.5]nonan-2,7-dione (18) as a white solid (32 mg, 62%).

Rf=0.10 (TLC, hexane-AcOEt, 3:1), 0.55 (TLC, hexane-AcOEt, 1:1); yield, 62%; white solid; ¹H-NMR (300 MHz, CDCl₃): δ 7.43-7.39 (2H, m, Ph), 7.34-7.32 (3H, m, Ph), 5.11 (1H, part A syst. AB, J=10.1 Hz, OCH₂Ph), 5.05 (1H, part B syst. AB, J=10.1 Hz, OCH₂Ph), 4.74 (1H, dd, J=3.6, 3.4 Hz, H-8), 4.44 (1H, part A syst. AB, J=3.2 Hz, H-6), 4.26 (1H, part B syst. AB, J=3.2 Hz, H-5), 4.17 (1H, dd, J=3.6, 1.6 Hz, H-9), 3.52 (1H, d, J=3.4 Hz, HO—C (8)), 3.46 (1H, part A syst. AB, J=13.7 Hz, H-3), 2.74 (1H, s, HO—C (9)), 2.38 (1H, part B syst. AB, J=13.7 Hz, H-3), 0.85 (9H, s, C(CH₃)₃), 0.12 (3H, s, SiCH₃), 0.08 (9H, s, Si(CH₃)₃), 0.00 (3H, s, SiCH₃); ¹³C-NMR (75 MHz, CDCl₃): δ 205.7, 165.3, 135.2, 129.3, 129.0, 128.6, 128.3, 78.7, 77.8, 75.4, 72.1, 67.6, 66.9, 37.5, 25.5, 18.0, 0.3, −5.0, −5.2; IR (NaCl, CCl₄): ν 3435, 2955, 2891, 2858, 1761, 1740, 1471, 1253, 1154, 1139, 1109, 887, 884, 780 cm⁻¹; LRMS (API-ES⁺): m/z 1041 (2M+Na)⁺, 532 (M+Na)⁺, 510 (M+H)⁺.

### Example 18

### Preparation of rac-(4R,5S,6S,7R)-1-benzyloxy-6-tert-butyldimethylsilyloxy-7,10-epoxy-7-methyl-5-trimethylsilyloxy-1-azaspiro[3.5]nona-8-en-2-one (20)

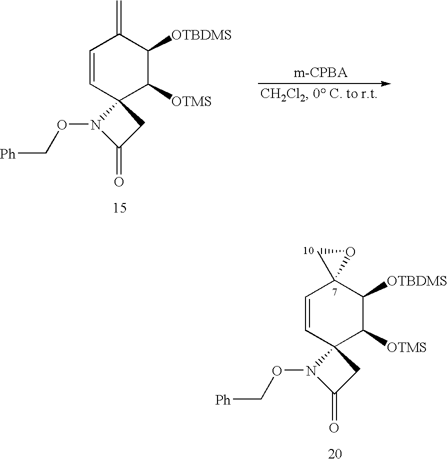

To a stirred solution of rac-(4R,5S,6S)-1-benzyloxy-6-tert-butyldimethylsilyloxy-7-methyl-5-trimethylsilyloxy-1-azaspiro[3.5]nona-8-en-2-one (15) (200 mg, 0.422 mmol) in CH₂Cl₂ (6 ml) was added at 0° C. 3-chloroperoxybenzoic acid (109 mg, 0.633 mmol). The mixture was stirred for 30 min at this temperature and then for 7 h at room temperature. This mixture was diluted in CH₂Cl₂ (3 ml), and washed with NaHCO₃ sat. (3×3 ml) and NaCl sat. (1×3 ml). The organic phase was dried over Na₂SO₄, filtered and concentrated under reduced pressure. The residue was purified by silica gel column chromatography (hexane-AcOEt, 8:1) to give rac-(4R,5S,6S,7R)-1-benzyloxy-6-tert-butyldimethylsilyloxy-7,10-epoxy-7-methyl-5-trimethylsilyloxy-1-azaspiro[3.5]nona-8-en-2-one (20) as a colourless oil (170 mg, 82%).

R_{f}=0.50 (TLC, hexane-AcOEt, 3:1); yield, 82%; colourless oil; ¹H-NMR (200 MHz, CO(CD₃)₂): δ 7.44-7.35 (5H, m, Ph), 5.02 (1H, part A syst. AB, J=11.1 Hz, OCH₂Ph), 4.93 (1H, part B syst. AB, J=11.1 Hz, OCH₂Ph), 4.34 (1H, br. s, H-5 or H-6), 3.58 (1H, br. s, H-6 or H-5), 3.17 (1H, m, H-3), 2.87 (2H, s, H-10), 2.44 (1H, d, J=14.1 Hz, H-3), 0.87 (9H, s, C(CH₃)₃), 0.16 (9H, s, Si(CH₃)₃), 0.02 (3H, s, SiCH₃), 0.01 (3H, s, SiCH₃); IR (NaCl, CCl₄): ν 3028, 2957, 2891, 2854, 1781, 1494, 1472, 1407, 1362, 1255, 1151, 1100, 1055, 994, 881, 842, 803, 778, 753, 666 cm⁻¹; LRMS (API-ES⁺): m/z 1003 (2M+Na)⁺, 513 (M+Na)⁺, 490 (M+H)⁺.

### Example 19

### Preparation of rac-(4R,5S,6S,8S,9S)-1-benzyloxy-6-tert-butyldimethylsilyloxy-9-hydroxy-7-methylen-5-trimethylsilyloxy-1-azaspiro[3.5]nonan-2-on-10,8-carbolactone (19)

### Method A.

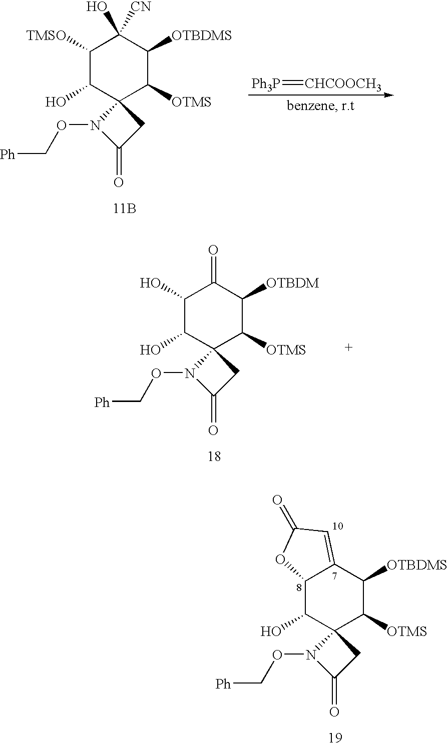

To a solution of rac-(4R,5S,6S,7S,8S,9S)-1-benzyloxy-6-tert-butyldimethylsilyloxy-7-cyano-7,9-dihydroxy-5,8-bis(trimethylsilyloxy)-1-azaspiro[3.5]nonan-2-one (11B) (58 mg, 0.095 mmol) in benzene (1.0 ml) was added at room temperature a solution of methyl (triphenylphosphoranylidene)acetate (76 mg, 0.228 mmol) in benzene (2 ml). The mixture was stirred to reflux for 11 h. The reaction was quenched with Na₂HPO₄ 0.1 M buffer (3 ml) and AcOEt (3 ml). The layers were separated and aqueous phase was extracted with AcOEt (3×6 ml). The combined extracts were dried over Na₂SO₄, filtered and concentrated under reduced pressure. The residue was purified by silica gel column chromatography (hexane-AcOEt, 4:1) to give rac-(4R,5S,6S,8S,9S)-1-benzyloxy-6-tert-butyldimethylsilyloxy-8,9-dihydroxy-5-trimethylsilyloxy-1-azaspiro[3.5]nonan-2,7-dione (18) as a white solid (19 mg, 38%) and rac-(4R,5S,6S,8S,9S)-1-benzyloxy-6-tert-butyldimethylsilyloxy-9-hydroxy-7-methylen-5-trimethylsilyloxy-1-azaspiro[3.5]nonan-2-on-10,8-carbolactone (19) as a white solid (10 mg, 20%).

### Method B.

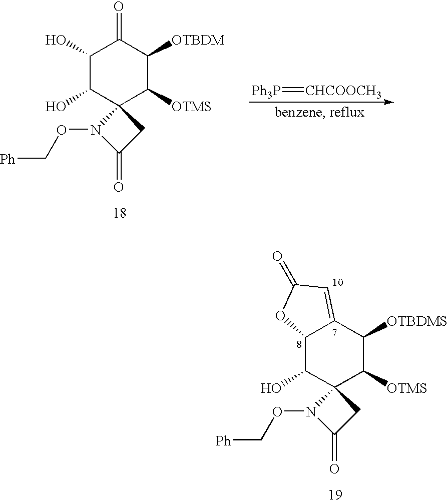

To a solution of rac-(4R,5S,6S,8S,9S)-1-benzyloxy-6-tert-butyldimethylsilyloxy-8,9-dihydroxy-5-trimethylsilyloxy-1-azaspiro[3.5]nonan-2,7-dione (18) (59 mg, 0.116 mmol) in benzene (1 ml) was added at room temperature a solution of methyl (triphenylphosphoranylidene)acetate (124 mg, 0.370 mmol) in benzene (3 ml). The mixture was stirred to reflux for 15 h. The reaction was quenched with Na₂HPO₄ 0.1M buffer (3 ml) and AcOEt (3 ml). The layers were separated and aqueous phase was extracted with AcOEt (3×6 ml). The combined extracts were dried over Na₂SO₄, filtered and concentrated under reduced pressure. The residue was purified by silica gel column chromatography (hexane-AcOEt, 3:1) to give rac-(4R,5S,6S,8S,9S)-1-benzyloxy-6-tert-butyldimethylsilyloxy-9-hydroxy-7-methylen-5-trimethylsilyloxy-1-azaspiro[3.5]nonan-2-on-10,8-carbolactone (19) as a white solid (44 mg, 71%).

Rf=0.14 (TLC, hexane-AcOEt, 3:1), 0.66 (TLC, hexane-AcOEt, 1:1); yield, 71%; white solid; ¹H-NMR (300 MHz, CDCl₃): δ 7.42-7.38 (2H, m, Ph), 7.36-7.33 (3H, m, Ph), 6.00 (1H, d, J=1.5 Hz, H-10), 5.09 (1H, dd, J=3.6, 1.5 Hz, H-8), 5.08 (1H, part A syst. AB, J=10.2 Hz, OCH₂Ph), 5.03 (1H, part B syst. AB, J=10.2 Hz, OCH₂Ph), 4.77 (1H, d, J=3.2 Hz, H-5), 4.32-4.27 (2H, m, H-9 and H-6), 3.46 (1H, part A syst. AB, J=13.8 Hz, H-3), 2.37 (1H, d, J=2.9 Hz, HO—C (9)), 2.32 (1H, part B syst. AB, J=13.8 Hz, H-3), 0.85 (9H, s, C(CH₃)₃), 0.10 (3H, s, SiCH₃), 0.09 (9H, s, Si(CH₃)₃), 0.01 (3H, s, SiCH₃); ¹³C-NMR (75 MHz, CDCl₃): δ 165.4, 165.1, 135.2, 129.4, 128.8, 128.4, 115.8, 78.8, 78.6, 72.7, 69.5, 67.1, 66.9, 37.6, 25.5, 18.0, 0.4, −4.9; IR (KBr): ν 3435, 2949, 2927, 2855, 1779, 1747, 1631, 1248, 1129, 1101, 840, 616 cm⁻¹; LRMS (API-ES): m/z 1088 (2M+Na)⁺, 556 (M+Na)⁺, 533 (M+H)⁺.

### Example 20

### Preparation of rac-(4R,5S,6S)-1-benzyloxy-6-tert-butyldimethylsilyloxy-7-methylen-5-trimethylsilyloxy-1-azaspiro[3.5]nona-8-en-2-on-10,8-carbolactone (21)

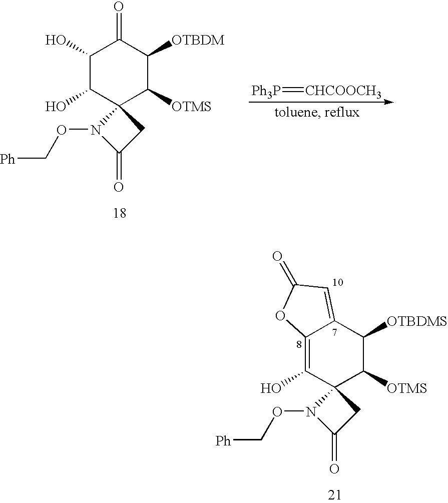

To a solution of rac-(4R,5S,6S,8S,9S)-1-benzyloxy-6-tert-butyldimethylsilyloxy-8,9-dihydroxy-5-trimethylsilyloxy-1-azaspiro[3.5]nonan-2,7-dione (18) (19 mg, 0.037 mmol) in toluene (0.3 ml) was added at room temperature a solution of methyl (triphenylphosphoranylidene)acetate (93 mg, 0.277 mmol) in toluene (0.7 ml). The resulting mixture was stirred at reflux for 24 h and then concentrated under reduced pressure. The residue was purified by silica gel column chromatography (hexane-AcOEt, 6:1) to give rac-(4R,5S,6S)-1-benzyloxy-6-tert-butyldimethylsilyloxy-7-methylen-5-trimethylsilyloxy-1-azaspiro[3.5]nona-8-en-2-on-10,8-carbolactone (21) as a yellow oil (44 mg, 71%).

Rf=0.55 (TLC, hexane-AcOEt, 3:1); yield, 26%; yellow oil; ¹H-NMR (200 MHz, CDCl₃): δ 7.35-7.26 (5H, m, Ph), 5.91 (1H, m, H-9 or H-10), 4.99 (1H, part A syst. AB, J=11.5 Hz, OCH₂Ph), 4.99 (1H, d, J=1.8 Hz, H-10 or H-9), 4.87 (1H, part B syst. AB, J=11.5 Hz, OCH₂Ph), 4.73 (1H, d, J=2.6 Hz, H-5 or H-6), 3.97 (1H, d, J=2.6 Hz, H-6 or H-5), 3.42 (1H, part A syst. AB, J=13.9 Hz, H-3), 2.47 (1H, part B syst. AB, J=13.9 Hz, H-3), 0.78 (9H, s, C(CH₃)₃), 0.20 (3H, s, SiCH₃), 0.12 (9H, s, Si(CH₃)₃), 0.07 (3H, s, SiCH₃); LRMS (API-ES⁺): m/z 538 (M+Na)⁺, 516 (M+H)⁺.

### Example 21

### Preparation of rac-(4R,5S,6S,7R)-1-benzyloxy-6-tert-butyldimethylsilyloxy-7-hydroxy-7-ethoxycarbonylmethyl-5-trimethylsilyloxy-1-azaspiro[3.5]nona-8-en-2-one (22)

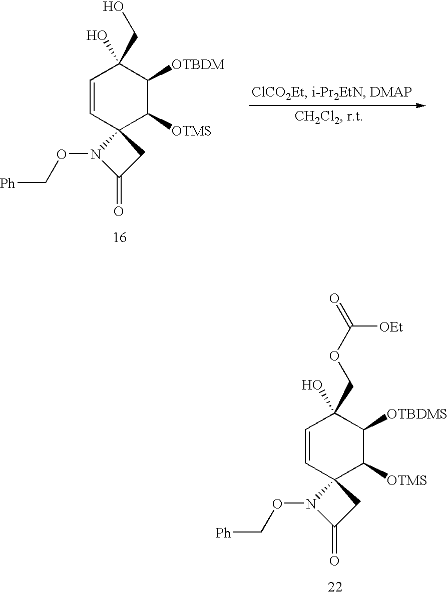

To a solution of rac-(4R,5S,6S,7R)-1-benzyloxy-6-tert-butyldimethylsilyloxy-7-hydroxy-7-hydroxymethyl-5-trimethylsilyloxy-1-azaspiro[3.5]nona-8-en-2-one (16) (52 mg, 0.102 mmol) and DMAP (1.3 mg, 0.010 mmol) in CH₂Cl₂ (1.5 ml) was added at 0° C. N-ethyldiisopropylamine (42 μl, 0.245 mmol) and ethyl chloroformate (12 μl, 0.122 mmol). The resulting mixture was stirred at room temperature until the reaction was complete (16 h, TLC monitoring, hexane-AcOEt, 1:1). The reaction was quenched with H₂O (drops), AcOEt (1.5 ml) and saturated NH₄Cl/NH₄OH solution (1.5 ml). The layers were separated and aqueous phase was extracted with AcOEt (3×3 ml). The combined extracts were washed with saturated NH₄Cl/NH₄OH solution (5 ml), dried over Na₂SO₄, filtered and concentrated under reduced pressure to give rac-(4R,5S,6S,7R)-1-benzyloxy-6-tert-butyldimethylsilyloxy-7-hydroxy-7-ethoxycarbonylmethyl-5-trimethylsilyloxy-1-azaspiro[3.5]nona-8-en-2-one (22) as a low melting point pale yellow solid (50 mg, 85%).

Rf=0.32 (TLC, hexane-AcOEt, 3:1), 0.73 (TLC, hexane-AcOEt, 1:1); yield, 85%; pale yellow solid; ¹H-NMR (200 MHz, CDCl₃): δ 7.42-7.29 (5H, m, Ph), 5.48 (1H, d, J=10.1 Hz, H-8 or H-9), 5.42 (1H, m, H-8 or H-9), 4.98 (1H, part A syst. AB, J=11.3 Hz, OCH₂Ph), 4.85 (1H, part B syst. AB, J=11.3 Hz, OCH₂Ph), 4.55 (1H, br. s, H-5 or H-6 or H-10), 4.22 (1H, q, J=14.4, 7.1 Hz, OCH₂CH₃), 4.22 (1H, q, J=14.2, 7.1 Hz, OCH₂CH₃), 4.13 (1H, br. s, H-6 or H-0 or H-5), 3.88 (1H, br. s, H-0 or H-5 or H-6), 3.23 (1H, part A syst. AB, J=13.4 Hz, H-3), 2.27 (1H, br. s, H-10 or H-5 or H-6), 2.26 (1H, part B syst. AB, J=13.4 Hz, H-3), 1.32 (3H, t, J=7.1 Hz, OCH₂H₃), 0.80 (9H, s, C(CH₃)₃), 0.18 (9H, s, Si(CH₃)₃), 0.15 (3H, s, SiCH₃), 0.06 (3H, s, SiCH₃); LRMS (API-ES): m/z 1181 (2M+Na)⁺, 602 (M+Na)⁺, 580 (M+H)⁺.

### Example 22

### Preparation of rac-(4R,5S,6S,7R)-1-benzyloxy-6-tert-butyldimethylsilyloxy-7-methyl-5-trimethylsilyloxy-7,10-phenylboranediyldioxy-1-azaspiro[3.5]nona-8-en-2-one (23)

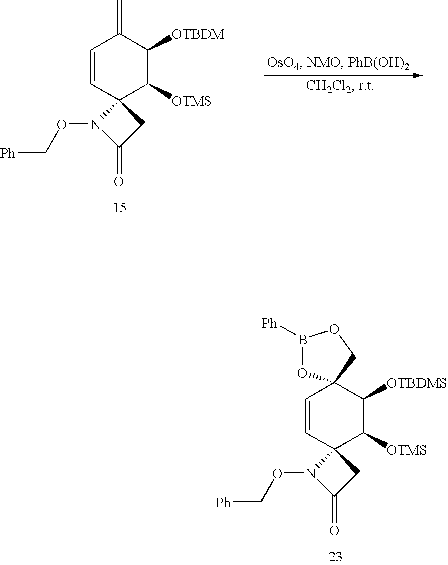

To a solution of N-methylmorpholine N-oxide (18 mg, 0.149 mmol) and phenylboric acid (19 mg, 0.149 mmol) in CH₂Cl₂ (0.25 ml) was added sequentially at room temperature osmium tetroxide (32 μl, 0.5 mM solution in CH₂Cl₂, 0.0025 mmol) and a solution of rac-(4R,5S,6S)-1-benzyloxy-6-tert-butyldimethylsilyloxy-7-methylen-5-trmethylsilyloxy-1-azaspiro[3.5]nona-8-en-2-one (15) (59 mg, 0.124 mmol) in CH₂Cl₂ (0.5 ml). The resulting mixture was stirred at room temperature until the reaction was complete (2 h, TLC monitoring, hexane-AcOEt, 3:1), and then quenched with 10% aqueous Na₂S₂O₃ solution (0.5 ml). After 15 min, the mixture was extracted with AcOEt (3×2 ml) and the combined organic extracts were dried over Na₂SO₄, filtered and concentrated under reduced pressure. The residue was purified by silica gel column chromatography (hexane-AcOEt, 5:1) to give rac-(4R,5S,6S,7R)-1-benzyloxy-6-tert-butyldimethylsilyloxy-7-methyl-5-trimethylsilyloxy-7,10-phenylboranediyldioxy-1-azaspiro[3.5]nona-8-en-2-one (23) as a low melting point white solid (33 mg, 45%).

Rf=0.40 (TLC, hexane-AcOEt, 3:1); yield, 45%; white solid; ¹H-NMR (200 MHz, CDCl₃): δ 7.84 (2H, dd, J=8.1, 1.5 Hz, Ph), 7.56-7.30 (8H, m, Ph), 6.91, 5.52 (1H, m), 5.52 (1H, m), 5.07 (1H, part A syst. AB, J=10.1 Hz, OCH₂Ph), 4.93 (1H, part B syst. AB, J=10.1 Hz, OCH₂Ph), 4.71 (1H, br. s), 4.60-4.30 (1H, m), 4.10-3.80 (2H, m), 3.26, 2.33 (1H, br. s), 2.59 (1H, br. s), 0.80 (9H, s, C(CH₃)₃), 0.17 (9H, s, Si(CH₃)₃), 0.10 (3H, s, SiCH₃), 0.07 (3H, s, SiCH₃).

### Example 23

### Preparation of rac-(4R,5S,6S,7R)-1-benzyloxy-6,7-bis(tert-butyldimethylsilyloxy)-7-hydroxy-5-trimethylsilyloxy-1-azaspiro[3.5]nona-8-en-2-one (24)

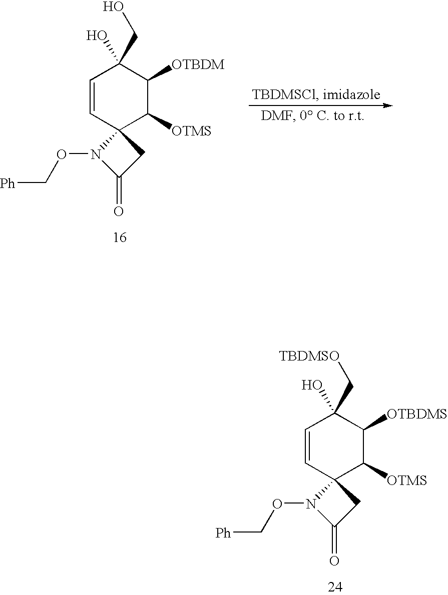

To a solution of rac-(4R,5S,6S,7R)-1-benzyloxy-6-tert-butyldimethylsilyloxy-7-hydroxy-7-hydroxymethyl-5-trimethylsilyloxy-1-azaspiro[3.5]nona-8-en-2-one (16) (50 mg, 0.098 mmol) and imidazole (8 mg, 0.118 mmol) in DMF (0.25 ml) was added at 0° C. a solution of tert-butyldimethylsilyl chloride (18 mg, 0.118 mmol) in DMF (0.25 ml). After 5 h at room temperature, the reaction was quenched with H₂O (1 ml) and the mixture extracted with AcOEt (3×2 ml). The combined extracts were washed with saturated aqueous CuSO₄ solution (2×3 ml) and brine (2×3 ml), dried over Na₂SO₄, filtered and concentrated under reduced pressure. The residue was purified by silica gel column chromatography (hexane-AcOEt, 6:1) to give rac-(4R,5S,6S,7R)-1-benzyloxy-6,7-bis(tert-butyldimethylsilyloxy)-7-hydroxy-5-trimethylsilyloxy-1-azaspiro[3.5]nona-8-en-2-one (24) as a white solid (50 mg, 82%).

Rf=0.69 (TLC, hexane-AcOEt, 3:1); yield, 82%; white solid; ¹¹H-NMR (200 MHz, CDCl₃): δ 7.44-7.27 (5H, m, Ph), 5.55 (2H, m, H-8 and H-9), 4.97 (1H, part A syst. AB, J=11.1 Hz, OCH₂Ph), 4.88 (1H, part B syst. AB, J=11.1 Hz, OCH₂Ph), 4.62 (1H, br. s, H-5 or H-6), 3.81 (1H, br. s, H-6 or H-5), 3.62 (1H, part A syst. AB, J=9.6 Hz, OCH₂CH₃), 3.54 (1H, part B syst. AB, J=9.6 Hz, OCH₂CH₃), 3.23 (1H, part A syst. AB, J=13.6 Hz, H-3), 2.94 (1H, br. s, OH), 2.26 (1H, part B syst. AB, J=13.6 Hz, H-3), 1.32 (3H, t, J=7.1 Hz, OCH₂CH₃), 0.92 (9H, s, C(CH₃)₃), 0.81 (9H, s, C(CH₃)₃), 0.17 (9H, s, Si(CH₃)₃), 0.14 (3H, s, SiCH₃), 0.09 (6H, s, SiCH₃), 0.06 (3H, s, SiCH₃).

### Example 24

### Preparation of rac-(4R,5S,6S,7S,8R,9R)-1-benzyloxy-8,9-dihydroxy-5,6-(1,1,3,3-tetraisopropyldisilydioxy)-10-[2-(4-methoxyphenyl)acetoxy]-1-azaspiro[3.5]nona-2-one (25)

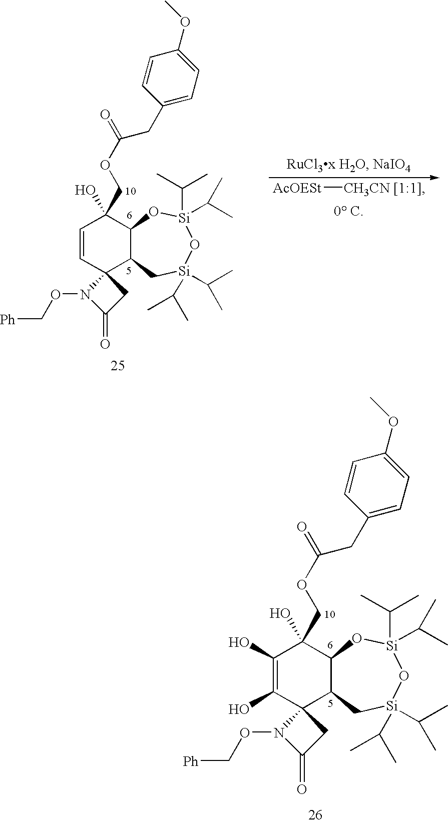

To a solution of rac-(4R,5S,6S,7R)-1-benzyloxy-5,6-(1,1,3,3-tetraisopropyldisilydioxy)-10-[2-(4-methoxyphenyl)acetoxy]-1-azaspiro[3.5]nona-8-en-2-one (25) (50 mg, 0.070 mmol) in AcOEt (0.6 ml) and CH₃CN (0.6 ml) was added at 0° C. with vigorous stirring a solution of ruthenium (III) chloride hydrate (4 mg, 0.017 mmol) and sodium periodate (18 mg, 0.084 mmol) in H₂O (0.2 ml). After 5 min the reaction the mixture was quenched with saturated aqueous sodium bisulfite solution (1.5 ml) and extracted with AcOEt (3×2 ml). The combined organic extracts were dried over Na₂SO₄, filtered and concentrated under reduced pressure. The residue was purified by silica gel column chromatography (hexane-AcOEt, 3:2) to give rac-(4R,5S,6S,7S,8R,9R)-1-benzyloxy-8,9-dihydroxy-5,6-(1,1,3,3-tetraisopropyldisilydioxy)-10-[2-(4-methoxyphenyl)acetoxy]-1-azaspiro[3.5]nona-2-one (126) as a white solid (43 mg, 83%).

Rf=0.43 (TLC, hexane-AcOEt, 2:1); yield, 83%; white solid; ¹H-NMR (300 MHz, CDCl₃): δ 7.44-7.31 (5H, m, Ph), 7.16 (2H, part A syst. AB, J=8.8 Hz, CH₃OC₆H₄), 6.87 (2H, part B syst. AB, J=8.8 Hz, CH₃OC₆H₄), 5.06 (1H, part A syst. AB, J=11.1 Hz, OCH₂Ph), 4.99 (1H, part B syst. AB, J=11.1 Hz, OCH₂Ph), 4.60 (1H, part A syst. AB, J=11.8 Hz, H-10), 4.51 (1H, d, J=2.9 Hz), 4.33 (1H, part B syst. AB, J=11.8 Hz, H-10), 4.28 (1H, dd, J=2.9, 1.5 Hz), 3.99 (1H, d, J=2.9 Hz), 3.80 (3H, s, OCH₃), 3.77 (1H, m), 3.63 (2H, s, CH₂CO₂CH₂), 2.97 (1H, d, J=10.0 Hz), 2.85 (1H, part A syst. AB, J=13.3 Hz, H-3), 2.80 (1H, part B syst. AB, J=13.3 Hz, H-3), 2.51 (1H, br. s, OH), 1.12-0.98 (28H, m, (i-Pr)₂Si); ¹³C-NMR (75 MHz, CDCl₃): δ 172.7, 166.0, 158.9, 135.7, 130.1, 128.9, 128.8, 128.6, 125.1, 114.2, 78.8, 75.6, 74.5, 72.6, 69.6, 68.9, 66.7, 65.7, 55.2, 40.3, 34.2, 17.6, 17.2, 17.1, 17.0, 16.9, 14.1, 13.8, 13.1, 13.0; IR (NaCl, CCl₄): ν 3435, 2946, 2868, 1747, 1613, 1514, 1464, 1249, 1151, 1105, 1133, 1006, 884, 754 cm⁻¹; LRMS (API-ES⁺): m/z 1513 (2M+Na)⁺, 769 (M+Na)⁺, 746 (M+H)⁺.

1. A compound of formula I: 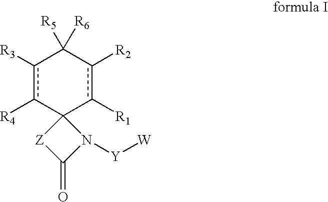
wherein R₁, R₂, R₃ and R₄ are each independently selected from H, OH, halo, OPR, ═0, cyano, substituted or unsubstituted alkyl, substituted or unsubstituted aryl, substituted or unsubstituted heterocyclyl, substituted or unsubstituted alkoxy, substituted or unsubstituted aryloxy, substituted or unsubstituted amino or halogen,
R₅ and R₆ together are =0 or —O—(CH₂)ₘ—, wherein m is selected from 1, 2, 3, 4 or 5;
or R₅ is selected from H, OH, OPR and R₆ is selected from hydrogen, cyano, substituted or unsubstituted alkyl, substituted or unsubstituted cycloalkyl, substituted or unsubstituted alkenyl, substituted or unsubstituted alkinyl, substituted or unsubstituted aryl, substituted or unsubstituted heterocyclyl,
with the proviso that at least one of R₁, R₂, R₃, R₄ or R₅ is OH, substituted or unsubstituted alkoxy, substituted or unsubstituted aryloxy or OPR;
PR is an hydroxyl protecting group that can be the same or different on each of R₁, R₂, R₃, R₄ or R₅ and that can simultaneously protect 1, 2 or 3 hydroxy groups;
the dotted line represents a single or double bond, with the proviso that when both R₁ and R₂ or R₃ and R₄ are H then there is a double bond between the two C to which the H are linked;
Z is —(CRaRb)ₙ— or —CH₂—(CRaRb)— or —(CRaRb)—CH₂— or —CH₂—(CRaRb)—CH₂—, or —(CH₂)₂—(CRaRb)— or —(CRaRb)-(CH₂)₂— wherein n is a number selected from 1, 2 or 3 and Ra and Rb are each independently selected from hydrogen, substituted or unsubstituted alkyl, substituted or unsubstituted cycloalkyl, substituted or unsubstituted alkenyl,
substituted or unsubstituted aryl, substituted or unsubstituted heterocyclyl, substituted or unsubstituted alkoxy, substituted or unsubstituted aryloxy, substituted or unsubstituted amino or halogen;
Y is selected from —O—, —S—, —NRa— or —C(O)—, wherein Ra is as previously defined;
W is a group comprising at least a group selected from substituted or unsubstituted aryl, substituted or usubstituted heterocyclyl, substituted or unsubstituted alkenyl; or
a salt, complex or solvate thereof.

2. A compound as defined in claim 1 wherein W is selected from substituted or unsubstituted arylalkyl, substituted or unsubstituted heterocyclylalkyl, substituted or unsubstituted alkenyl.

3. A compound as defined in claim 2 wherein W is arylalkyl.

4. A compound as defined in claim 3 wherein W is benzyl.

5. A compound as defined in any of claim 1 wherein W is —CRaRb-Q, wherein Ra and Rb are as previously defined and Q is substituted or unsubstituted aryl, substituted or unsubstituted heterocyclyl, substituted or unsubstituted alkenyl.

6. A compound as defined in claim 5 wherein Ra and Rb are different.

7. A compound as defined in claim 1 wherein Y is -0-.

8. A compound as defined in claim 1 wherein Z is —CRaRb—.

9. A compound as defined in claim 8 wherein Z is —CRaH—.

10. A compound as defined in 9 claim 1 wherein Z has a chiral center.

11. A compound according to claim 1 having formula II 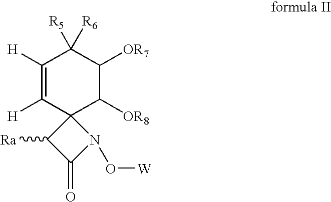
wherein
R₇ and R₈ are independently selected from H, substituted or unsubstituted alkyl or PR; W, Ra, R₅ and R₆ are as defined in claim 1.

12. A compound according to claim 1 having formula III 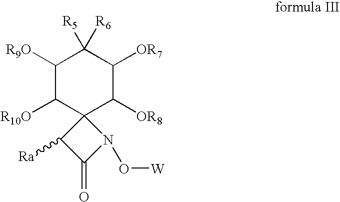
wherein R₇, R₈, R₉ and R₁₀ are each independently selected from H, substituted or unsubstituted alkyl or PR;
W, Ra, R₅ and R₆ are as defined in claim 1.

13. A compound according to claim 11 wherein there are at least 2 different protecting groups PR on R₅, R₇, R₈, R₉ and R₁₀.

14. A compound according to claim 1 which corresponds to any of the following formulae: 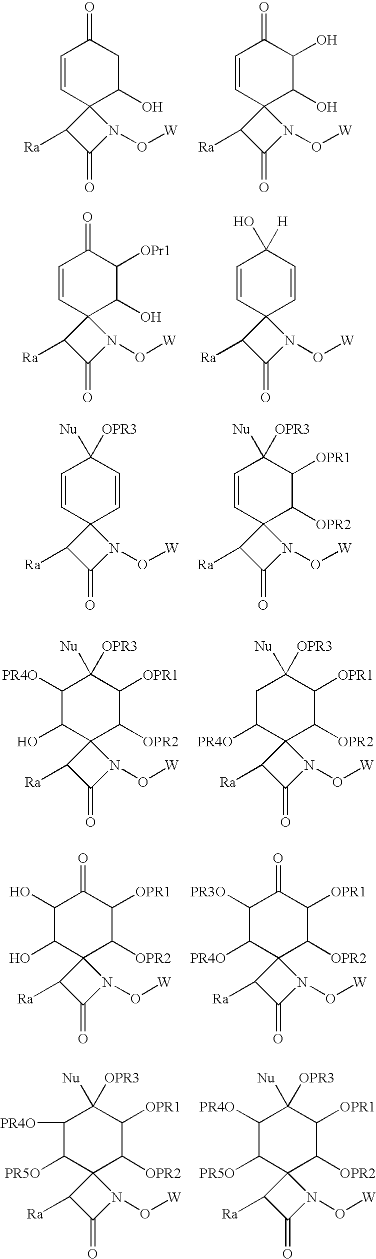 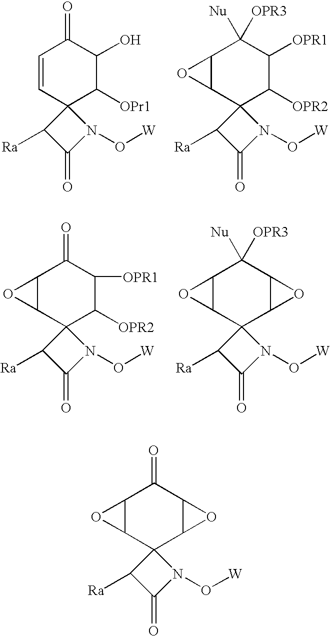
wherein W, PR and Ra are as above defined and wherein the protecting groups PR1-5 can be the same or different and can simultaneously protect 2 or 3 different hydroxy groups, and Nu is a nucleophilic group; their diastereoisomers, enantiomers and mixtures thereof.

15. A compound according to claim 14 which corresponds to any of the following formulae: 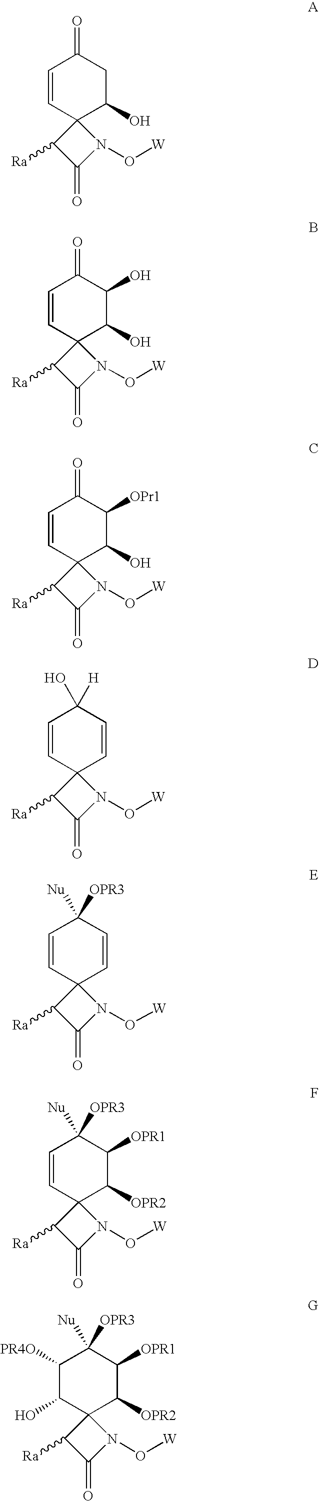 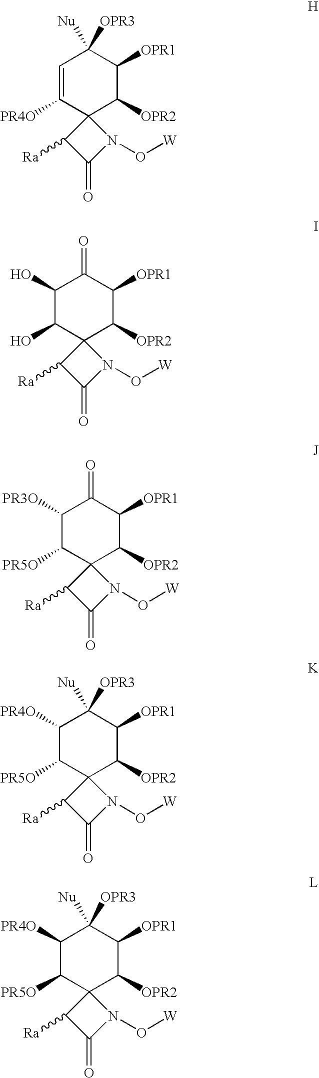
wherein W, PR and Ra are as above defined and wherein the protecting groups PR1-5 can be the same or different and can simultaneously protect 2 or 3 different hydroxy groups, and Nu is a nucleophilic group; their diastereoisomers, enantiomers and mixtures thereof.

16. A compound according to claim 1 which corresponds to any of the following formulae: 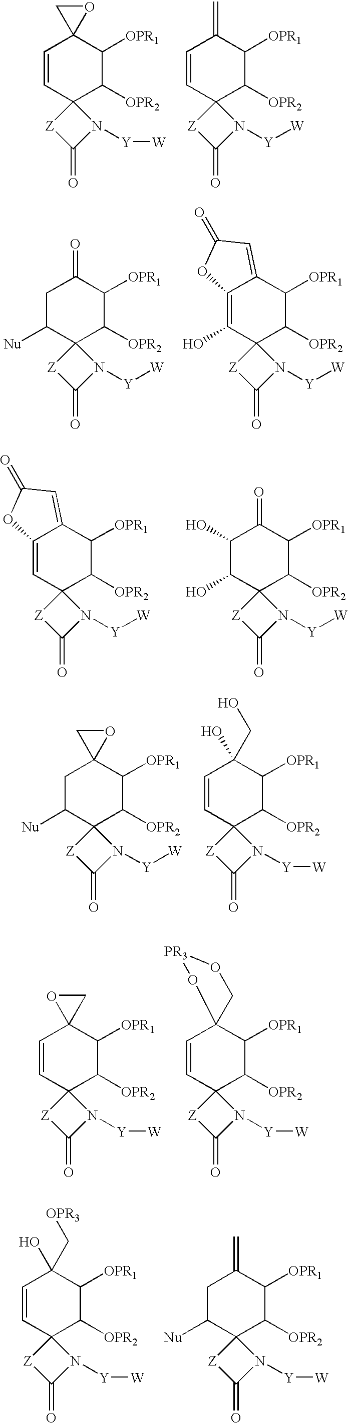 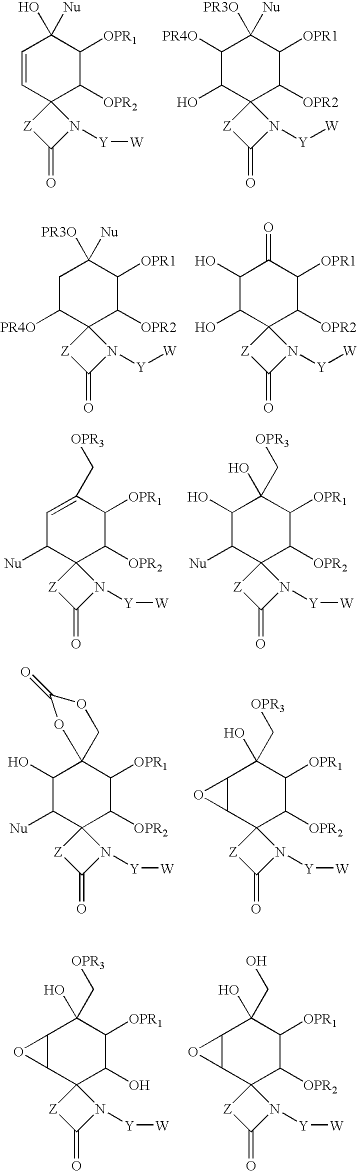
wherein W, Z, Y and PR are as above defined and wherein the protecting groups PR1-4 can be the same or different and can simultaneously protect 2 or 3 different hydroxy groups, and Nu is a nucleophilic group; their diastereoisomers, enantiomers and mixtures thereof.

17. A compound according to claim 16 which corresponds to any of the following formulae: 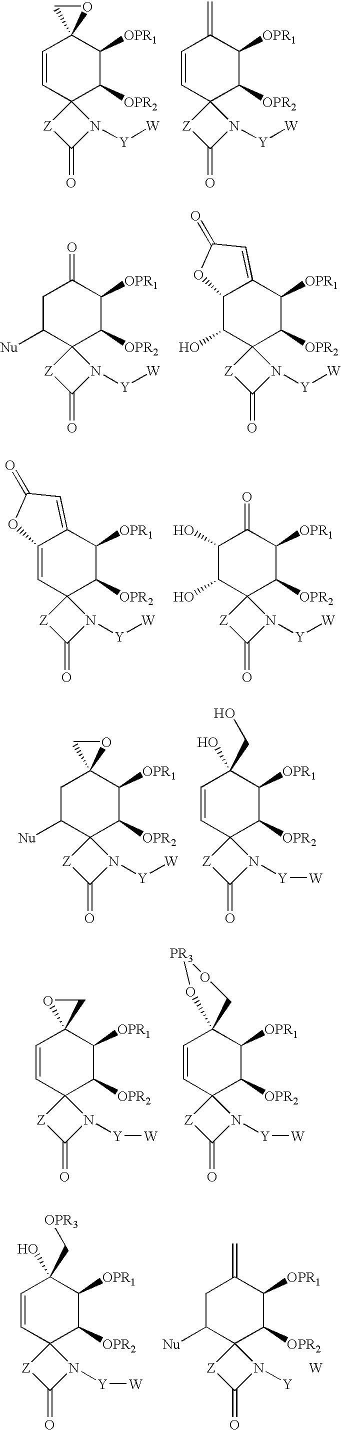 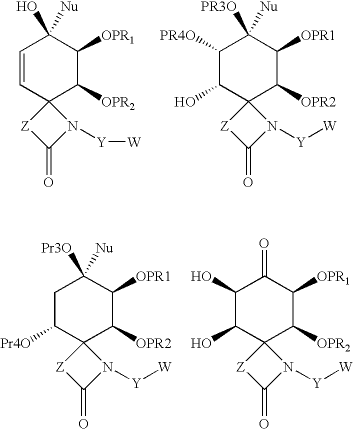
wherein W, Z, Y and PR are as above defined and wherein the protecting groups PR 1-4 can be the same or different and can simultaneously protect 2 or 3 different hydroxy groups, and Nu is a nucleophilic group; their diastereoisomers, enantiomers and mixtures thereof.

18. A compound according to claim 1 which corresponds to any of the following formulae: 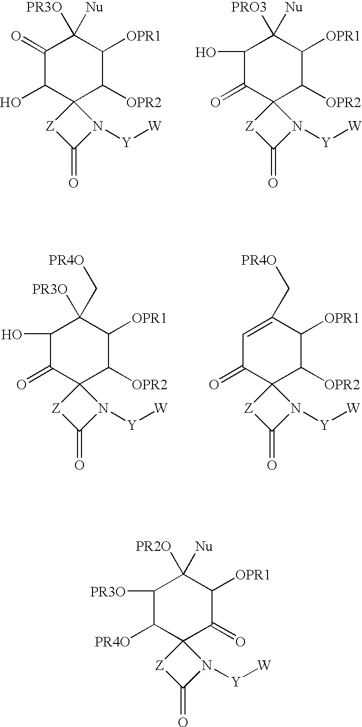
wherein W, Z, Y and PR are as above defined and wherein the protecting groups PR1-4 can be the same or different and can simultaneously protect 2 or 3 different hydroxy groups, and Nu is a nucleophilic group; their diastereoisomers, enantiomers and mixtures thereof.

19. A compound according to claim 16 wherein Z is —CHRa— wherein Ra is as above defined.

20. A compound according to claim 19 wherein Z has a chiral center.

21. A compound according to claim 16
wherein Y is —O—.

22. A compound according to claim 14 wherein Nu is selected from the group formed by hydrogen, cyano, halogen, substituted or unsubstituted alkyl, substituted or unsubstituted cycloalkyl, substituted or unsubstituted alkenyl, substituted or unsubstituted alkinyl, substituted or unsubstituted aryl, substituted or unsubstituted heterocyclyl.

23. A process for the preparation of a compound according to claim 1 which comprises in any order one or more of a step selected from the group consisting of:
a) hydroxylation, dihydroxylation or ketohydroxylation,
b) hydroxyl or carbonyl protection,
c) nucleophilic attack on the carbonyl group,
d) electrophilic attack on a double bond,
d) hydroxyl inversion,
e) allylic rearrangement,
applied to a compound of formula IV: 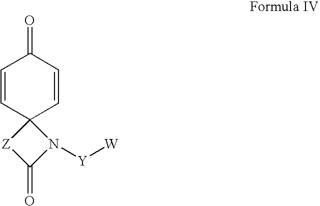
wherein Z, Y and W are as defined in claim 1.
